SEQUENCE LISTING (93 sequences)

SEQ ID NO: 1 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 2 - protein, 73 aa, Homo sapiens
KSMQVPFSRCCFSFAEQEIPLRAILCYRNTSSICSNEGLIFKLKRGKEACALDTVGWVQRHRKMLRHCPSKRK

SEQ ID NO: 3 - protein, 5 aa, Mus musculus
AYAMN

SEQ ID NO: 4 - protein, 19 aa, Mus musculus
RIRSKSNNYATYYADSVKD

SEQ ID NO: 5 - protein, 12 aa, Mus musculus
GGTYGSSSYFDY

SEQ ID NO: 6 - protein, 12 aa, Mus musculus
GGTYGSTSYFDY

SEQ ID NO: 7 - protein, 5 aa, Mus musculus
TYAMN

SEQ ID NO: 8 - protein, 19 aa, Mus musculus
RIRSKSNNYATYYADSVKA

SEQ ID NO: 9 - protein, 5 aa, Mus musculus
DYNMD

SEQ ID NO: 10 - protein, 17 aa, Mus musculus
AINPNNGGTGYTQKFKG

SEQ ID NO: 11 - protein, 8 aa, Mus musculus
RGVYMFAY

SEQ ID NO: 12 - protein, 16 aa, Mus musculus
RSSKSLLHSNGNTYLY

SEQ ID NO: 13 - protein, 7 aa, Mus musculus
RMSNLAS

SEQ ID NO: 14 - protein, 9 aa, Mus musculus
MQHLEYPFT

SEQ ID NO: 15 - protein, 16 aa, Mus musculus
KSSQSLLHSDGKTYLN

SEQ ID NO: 16 - protein, 7 aa, Mus musculus
LVSKLDS

SEQ ID NO: 17 - protein, 9 aa, Mus musculus
WQGTHFPYT

SEQ ID NO: 18 - protein, 123 aa, Mus musculus
EVQLVESGGGLVQPKGSLKLSCAASGFSFNAYAMNWVRQAPGKGLEWVARIRSKSNNYATYYADSVKDRFTISRDDSETMLYLQMNNLKTEDTAMYFCVRGGTYGSSSYFDYWGQGTTLTVSS

SEQ ID NO: 19 - protein, 112 aa, Mus musculus
DIVMTQAAPSVPVTPGESVSIPCRSSKSLLHSNGNTYLYWFLQRPGQSPQLLIYRMSNLASGVPDRFSGSGSGTAFTLRISRVEAEDVGVYYCMQHLEYPFTFGGGTKLQIR

SEQ ID NO: 20 - protein, 123 aa, Mus musculus
EVQLVESGGGLVQPKGSLKLSCAASGFSFNAYAMNWVRQAPGKGLEWVARIRSKSNNYATYYADSVKDRFIISRDDSESMLYLQMNNLKTEDTAMYFCVRGGTYGSTSYFDYWGQGTTLTVSS

SEQ ID NO: 21 - protein, 112 aa, Mus musculus
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGGGTKLEIK

SEQ ID NO: 22 - protein, 123 aa, Mus musculus
EVQLVESGGGLVQPKGSLKLSCAASGFSFNTYAMNWVRQAPGKGLEWVARIRSKSNNYATYYADSVKARFTISRDDSESMLYLQMNNLKTEDTAMYFCVRGGTYGSTSYFDYWGQGTTLTVSS

SEQ ID NO: 23 - protein, 112 aa, Mus musculus; identical to SEQ ID NO: 21

SEQ ID NO: 24 - protein, 117 aa, Mus musculus
EVQLQQSGPELVKPGSSVKISCKASGYTFTDYNMDWVKQSHGKSLEWIGAINPNNGGTGYTQKFKGKATLTVDKSSSTAFMELRSLTSEDSAVYYCARRGVYMFAYWGQGTLVTVSA

SEQ ID NO: 25 - protein, 112 aa, Mus musculus
DVVMTQTPLTLSVTIGQPASISCKSSQSLLHSDGKTYLNWLLQRPGQSPKRLIYLVSKLDSGVPDRFTGSGSGTDFTLKISRVEAEDLGVYYCWQGTHFPYTFGGGTKLEIK

SEQ ID NO: 26 - protein, 453 aa, synthetic construct
EVQLVESGGGLVQPKGSLKLSCAASGFSFNAYAMNWVRQAPGKGLEWVARIRSKSNNYATYYADSVKDRFTISRDDSETMLYLQMNNLKTEDTAMYFCVRGGTYGSSSYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 219 aa, synthetic construct
DIVMTQAAPSVPVTPGESVSIPCRSSKSLLHSNGNTYLYWFLQRPGQSPQLLIYRMSNLASGVPDRFSGSGSGTAFTLRISRVEAEDVGVYYCMQHLEYPFTFGGGTKLQIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 453 aa, synthetic construct
EVQLVESGGGLVQPKGSLKLSCAASGFSFNTYAMNWVRQAPGKGLEWVARIRSKSNNYATYYADSVKARFTISRDDSESMLYLQMNNLKTEDTAMYFCVRGGTYGSTSYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 219 aa, synthetic construct
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 447 aa, synthetic construct
EVQLQQSGPELVKPGSSVKISCKASGYTFTDYNMDWVKQSHGKSLEWIGAINPNNGGTGYTQKFKGKATLTVDKSSSTAFMELRSLTSEDSAVYYCARRGVYMFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 219 aa, synthetic construct
DVVMTQTPLTLSVTIGQPASISCKSSQSLLHSDGKTYLNWLLQRPGQSPKRLIYLVSKLDSGVPDRFTGSGSGTDFTLKISRVEAEDLGVYYCWQGTHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 107 aa, Homo sapiens [sequence omitted]

SEQ ID NO: 33 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 330 aa, Homo sapiens [sequence omitted]

SEQ ID NO: 35 - protein, 19 aa, Mus musculus
RIRTKSNNYATYYADSVKD

SEQ ID NO: 36 - protein, 19 aa, Mus musculus
RIRTKSNNYATFYADSVKD

SEQ ID NO: 37 - protein, 19 aa, Mus musculus
RIRTKSNNYATYYAASVKD

SEQ ID NO: 38 - protein, 19 aa, Mus musculus
RIRSKSNNFATYYADSVKD

SEQ ID NO: 39 - protein, 13 aa, Mus musculus
GGSGIKYVRYFDV

SEQ ID NO: 40 - protein, 13 aa, Mus musculus
GGSGIRYVKYFDV

SEQ ID NO: 41 - protein, 13 aa, Mus musculus
GGSGISYVRYFDV

SEQ ID NO: 42 - protein, 13 aa, Mus musculus
GGSGLNYVRYFDV

SEQ ID NO: 43 - protein, 13 aa, Mus musculus
GGSGLRYVRYFDV

SEQ ID NO: 44 - protein, 12 aa, Mus musculus
QTYGSRDYAMDY

SEQ ID NO: 45 - protein, 13 aa, Mus musculus
GGSGIRYVRYFDV

SEQ ID NO: 46 - protein, 16 aa, Mus musculus
RSSQSLVHSNGNTYLH

SEQ ID NO: 47 - protein, 16 aa, Mus musculus
RSSKSLQHSNGNIYLY

SEQ ID NO: 48 - protein, 7 aa, Mus musculus
KVSNRFS

SEQ ID NO: 49 - protein, 7 aa, Mus musculus
RMSDLAS

SEQ ID NO: 50 - protein, 10 aa, Mus musculus
CQSTHVPPYT

SEQ ID NO: 51 - protein, 10 aa, Mus musculus
SQSTHVPPYT

SEQ ID NO: 52 - protein, 10 aa, Mus musculus
SQNTHVPPYT

SEQ ID NO: 53 - protein, 124 aa, Mus musculus
EVQLVESGGGLVQPKGSLKLSCAASGFSFNTYAMNWVRQAPGKGLEWVARIRTKSNNYATYYADSVKDRFTISRDDSENILYLQMNNLKTEDTAMYYCVRGGSGIKYVRYFDVWGTGTTVTVSS

SEQ ID NO: 54 - protein, 124 aa, Mus musculus
EVQLVESGGGLVQPRGSLKLSCAASGFSFNAYAMNWVRQAPGKGLEWVARIRTKSNNYATYYADSVKDRFTISRDDSESMLYLQMINLKTEDTAMYYCVRGGSGIRYVKYFDVWGTGTTVTVSS

SEQ ID NO: 55 - protein, 124 aa, Mus musculus
EVQLVESGGGLVQPGGSLKLSCAASGFSFNAYAMNWVRQAPGKGLEWVARIRTKSNNYATYYADSVKDRFTISRDDSESMLYLQMINLKTEDTAMYYCVRGGSGIRYVKYFDVWGTGTTVTVSS

SEQ ID NO: 56 - protein, 124 aa, Mus musculus
EVQLVESGGGLVQPKGSLKLSCAASGFSFNTYAMNWVRQAPGKGLEWVARIRSKSNNYATYYADSVKDRFTISRDDSESMLYLQMNNLKTEDTAMYYCVRGGSGISYVRYFDVWGTGTTVTVSS

SEQ ID NO: 57 - protein, 124 aa, Mus musculus
EVQLVESGGGLVQPKGSLKLSCAASGFSFKTYAMNWVRQAPGKGLEWVARIRTKSNNYATYYADSVKDRFTISRDDSETMLYLQMNNLKTEDTAMYYCVRGGSGLNYVRYFDVWGTGTTVTVSS

SEQ ID NO: 58 - protein, 124 aa, Mus musculus
EVQLVESGGGLVQPKGSLKLSCAASGFSFNTYAMNWVRQAPGKGLEWVARIRTKSNNYATYYADSVKDRFTISRDDSESMLYLQMNNLKTEDTAMYYCVRGGSGLRYVRYFDVWGTGTTVTVSS

SEQ ID NO: 59 - protein, 124 aa, Mus musculus
EVQLVESGGGLVQPKGSLKLSCAASGFSFNTYAMNWVRQAPGKGLEWVARIRTKSNNYATYYADSVKDRFTISRDDSENMLYLQMNNLKTEDTAMYYCVRGGSGLRYVRYFDVWGTGTTVTVSS

SEQ ID NO: 60 - protein, 123 aa, Mus musculus
EVQLVESGGGLVQPKGSLKLSCAASGFSFNTYAMNWVRQAPGKGLDWVARIRSKSNNYATYYADSVKDRFTISRDDSESMLYLQMNNLKTEDTAMYFCVRQTYGSRDYAMDYWGQGTSVTVSS

SEQ ID NO: 61 - protein, 123 aa, Mus musculus
EVQLVESGGGLVQPKGSLKLSCAASGFSFNAYAMNWVRQAPGKGLDWVARIRSKSNNYATYYADSVKDRFTISRDDSESMLYLQMNNLKTEDTAMYFCVRQTYGSRDYAMDYWGQGTSVTVSS

SEQ ID NO: 62 - protein, 124 aa, Mus musculus
EVQLVESGGGLVQPKGSLKLSCAASGFSFNTYAMNWVRQAPGKGLEWVARIRSKSNNYATYYADSVKDRFTISRDDSESMLYLQMNNLKTEDTAMYYCVRGGSGIRYVRYFDVWGTGTTVTVSS

SEQ ID NO: 63 - protein, 123 aa, Mus musculus
EVQLVESGGGLVQPRGSLKLSCAASGFSFNAYAMNWVRQAPGKGLEWVARIRSKSNNFATYYADSVKDRFTISRDDSESMLYLQMNNLKTEDTAMYYCVRQTYGSRDYAMDYWGQGTSVTVSS

SEQ ID NO: 64 - protein, 124 aa, Mus musculus
EVQLVESGGGLVQPKGSLKLSCAASGFSFKTYAMNWVRQAPGEGLEWVARIRTKSNNYATYYADSVKDRFTISRDDSETMLYLQMNNLKTEDTAMYYCVRGGSGLNYVRYFDVWGPGTTVTVSS

SEQ ID NO: 65 - protein, 124 aa, Mus musculus
EVQLVESGGGLVQPKGSLKLSCAASGFSFNTYAMNWVRQAPGKGLEWVARIRTKSNNYATFYADSVKDRFTISRHDSESMLYLQMNNLKTEDTAMYYCVRGGSGIRYVRYFDVWGTGTTVTVSS

SEQ ID NO: 66 - protein, 124 aa, Mus musculus
EVQLVESGGGLVQPKGSLKLSCAASGFSFNTYAMNWVRQAPGKGLEWVARIRTKSNNYATYYAASVKDRFTISRDDSETMLYLQMNNLKTEDTAMYYCVRGGSGLNYVRYFDVWGTGTTVTVSS

SEQ ID NO: 67 - protein, 123 aa, Mus musculus
EVQLVESGGGLVQPKGSLKLSCAASGFSFNAYAMNWVRQAPGKGLEWVARIRSKSNNFATYYADSVKDRFTISRDDSESMLYLQMNNLKTEDTAMYYCVRQTYGSRDYAMDYWGQGTSVTVSS

SEQ ID NO: 68 - protein, 112 aa, Mus musculus
DIVMTQAAPSVPVTPGESVSISCRSSKSLLHSNGNTYLYWFLQRPGQSPQLLIYRMSNLASGVPERFSGSGSGSAFTLRISRVEAEDVGVYYCMQHLEYPFTFGSGTKLEIK

SEQ ID NO: 69 - protein, 112 aa, Mus musculus
DIVMTQAAPSVPVTPGESVSISCRSSKSLLHSNGNTYLYWFLQRPGQSPQLLIYRMSNLASGVPDRFSGSGSGSAFTLRISRVEAEDVGVYYCMQHLEYPFTFGSGTKLEIK

SEQ ID NO: 70 - protein, 112 aa, Mus musculus
DIVMTQATPSVPVTPGESVSISCRSSKSLLHSNGNTYLYWFLQRPGQSPQLLIYRMSNLASGVPERFSGSGSGSAFTLRVSRVEAEDVGVYYCMQHLEYPFTFGSGTKLEIK

SEQ ID NO: 71 - protein, 113 aa, Mus musculus
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCCQSTHVPPYTFGGGTKLEIK

SEQ ID NO: 72 - protein, 112 aa, Mus musculus
DIVMTQAAPSVPVTPGESVSISCRSSKSLQHSNGNIYLYWFLQRPGQSPQLLIYRMSNLASGVPDRFSGSGSGSAFTLRISRVEAEDVGVYYCMQHLEYPFTFGSGTKLEIK

SEQ ID NO: 73 - protein, 113 aa, Mus musculus
DVVMTQTPLSLPVSLGDRASISCRSSQSLVHSNGNTYLHWYLQKPGQSPRLLIYKVSNRFSGVPDRFSGSGSGTDFTLKISRVEAEDLGVYFCSQSTHVPPYTFGGGTKLEIK

SEQ ID NO: 74 - protein, 112 aa, Mus musculus
DIVMTQAAPSVLVTPGESVSFSCRSSKSLLHSNGNTYLYWFLQRPGQSPQLLIFRMSNLASGVPDRFSGSGSGSAFTLRISRVEAEDVGVYYCMQHLEYPFTFGSGTKLEIK

SEQ ID NO: 75 - protein, 112 aa, Mus musculus
DIVMTQAAPSVTVTPGESVSISCRSSKSLLHSNGNTYLYWFLQRPGQSPQLLIYRMSDLASGVPDRFSGSGSGSAFTLRISRVEAEDVGVYYCMQHLEYPFTFGSGTKLEIK

SEQ ID NO: 76 - protein, 112 aa, Mus musculus
DIVMTQAAPSVFVIPGESVSISCRSSKSLLHSNGNTYLYWFLQRPGQSPQLLIYRMSNLASGVPDRFSGSGSGSAFTLRISRVEAEDVGVYYCMQHLEYPFTFGSGTKLEIK

SEQ ID NO: 77 - protein, 113 aa, Mus musculus
DVVMTQTPLSLPVSLGDRASISCRSSQSLVHSNGNTYLHWYLQKPGQSPRLLIYKVSNRFSGVPDRFSGSGSGTDFTLKISRVEAEDLGVYFCSQNTHVPPYTFGGGTKLEIK

SEQ ID NO: 78 - protein, 454 aa, synthetic construct
EVQLVESGGGLVQPKGSLKLSCAASGFSFNTYAMNWVRQAPGKGLEWVARIRTKSNNYATYYAASVKDRFTISRDDSETMLYLQMNNLKTEDTAMYYCVRGGSGLNYVRYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 219 aa, synthetic construct
DIVMTQAAPSVFVIPGESVSISCRSSKSLLHSNGNTYLYWFLQRPGQSPQLLIYRMSNLASGVPDRFSGSGSGSAFTLRISRVEAEDVGVYYCMQHLEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 454 aa, synthetic construct
EVQLVESGGGLVQPKGSLKLSCAASGFSFNTYAMNWVRQAPGKGLEWVARIRTKSNNYATYYADSVKDRFTISRDDSESMLYLQMNNLKTEDTAMYYCVRGGSGLRYVRYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 219 aa, synthetic construct
DIVMTQATPSVPVTPGESVSISCRSSKSLLHSNGNTYLYWFLQRPGQSPQLLIYRMSNLASGVPERFSGSGSGSAFTLRVSRVEAEDVGVYYCMQHLEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 454 aa, synthetic construct
EVQLVESGGGLVQPKGSLKLSCAASGFSFNTYAMNWVRQAPGKGLEWVARIRTKSNNYATYYADSVKDRFTISRDDSENMLYLQMNNLKTEDTAMYYCVRGGSGLRYVRYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 219 aa, synthetic construct; identical to SEQ ID NO: 81

SEQ ID NO: 84 - protein, 453 aa, synthetic construct
EVQLVESGGGLVQPKGSLKLSCAASGFSFNAYAMNWVRQAPGKGLDWVARIRSKSNNYATYYADSVKDRFTISRDDSESMLYLQMNNLKTEDTAMYFCVRQTYGSRDYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 220 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCCQSTHVPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 453 aa, synthetic construct
EVQLVESGGGLVQPGGSLKLSCAASGFSFNAYAMNWVRQASGKGLEWVARIRSKSNNYATYYADSVKDRFTISRDDSKNTAYLQMNSLKTEDTAVYFCVRGGTYGSSSYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 219 aa, synthetic construct
DIVMTQSPLSLPVTPGEPASIPCRSSKSLLHSNGNTYLYWFLQKPGQSPQLLIYRMSNLASGVPDRFSGSGSGTAFTLKISRVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 453 aa, synthetic construct
EVQLVESGGGLVQPGGSLKLSCAASGFSFNAYAMNWVRQASGKGLEWVARIRSKSNNYATYYADSVKDRFTISRDDSENTAYLQMNSLKTEDTAVYFCVRGGTYGSSSYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 219 aa, synthetic construct
DIVMTQSPLSLPVTPGEPASISCRSSKSLLHSNGNTYLYWFLQKPGQSPQLLIYRMSNLASGVPDRFSGSGSGTAFTLKISRVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 454 aa, synthetic construct
EVQLVESGGGLVQPGGSLKLSCAASGFSFNTYAMNWVRQASGKGLEWVGRIRTKSNNYATYYAASVKDRFTISRDDSKNTAYLQMNSLKTEDTAVYYCTRGGSGLNYVRYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 219 aa, synthetic construct
DIVMTQTPPSLPVNPGEPASISCRSSKSLLHSNGNTYLYWYLQKPGQSPQLLIYRMSNLASGVPDRFSGSGSGSDFTLKISWVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 219 aa, synthetic construct
DIVMTQTPPSLPVNPGEPASISCRSSKSLLHSNANTYLYWYLQKPGQSPQLLIYRMSNLASGVPDRFSGSGSGSDFTLKISWVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 599 aa, synthetic construct
EVQLVESGGGLVQPGGSLKLSCAASGFSFNTYAMNWVRQASGKGLEWVGRIRTKSNNYATYYAASVKDRFTISRDDSKNTAYLQMNSLKTEDTAVYYCTRGGSGLNYVRYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGYTYSRHCLGWFRQAPGKGREAVSTIDSDGSTSYADSVKGRFTISRDNAKNTLYLQMNSLRPEDTAVYYCAIGPNPRYCSGAPNTRGAEHYFGYWGQGTLVTVSS

(19) United States
(12) Patent Application Publication
(10) Pub. No.: US 2026/0109770 A1
(43) Pub. Date: Apr. 23, 2026

(54) C-C CHEMOKINE RECEPTOR TYPE 8 (CCR8) ANTAGONIST ANTIBODIES

(71) Applicant: REMD Biotherapeutics Inc, Camarillo, CA (US)

(72) Inventors: Hai Yan, Thousand Oak, CA (US); Wuyi Wang, Newbury Park, CA (US); Kristopher Steward, Newbury Park, CA (US); John Zhang, Camarillo, CA (US)

(73) Assignee: REMD Biotherapeutics Inc, Camarillo, CA (US)

(21) Appl. No.: 19/117,038

(22) PCT Filed: Sep. 30, 2023
(86) PCT No.: PCT/US2023/034246

§ 371 Date: Mar. 29, 2025

Related U.S. Application Data
(60) Provisional application No. 63/412,465, filed on Oct. 2, 2022.

(51) Int. Cl. C07K 16/28

(52) U.S. Cl. CPC C07K 16/2866; C07K 16/2818; C07K 2317/24; C07K 2317/31; C07K 2317/33; C07K 2317/92

(57) ABSTRACT

This application provides isolated antibodies (Abs), e.g., monoclonal antibodies (mAbs), that bind specifically to C—C Motif Chemokine Receptor 8 (CCR8), and bispecific antibodies targeting CCR8 and CTLA-4, and methods for treating a cancer in a subject comprising administering to the subject an anti-CCR8 Ab as monotherapy or in combination with an anticancer agent such as an immune checkpoint inhibitor.

### RELATED PATENT APPLICATIONS

This application claims benefit of U.S. Provisional Application No. 63/412,465, filed on Oct. 2, 2022, incorporated in its entirety by reference herein.

### SEQUENCE LISTING

The contents of the electronic sequence listing (SeqListing-REMD CCR8.xml; Size: 91 Kilobytes; Production Date: Sep. 30, 2023) is herein incorporated by reference in its entirety.

### TECHNICAL FIELD

Two major populations of CD4+ regulatory T cells (Treg cells), defined by whether they express the forkhead box protein 3 transcription factor (Foxp3), are thought to play a key role in the maintenance of self-tolerance (Barsheshea et al., PNAS, 114 (23): 6086-6091, 2017). Both Foxp3+ and Foxp3− subtypes participate in the regulation of inflammatory autoimmunity and in the maintenance of self-tolerance by various mechanisms, including regulating the biological function of effector TH1 and TH17 CD4+ T cells (Id). Mechanisms utilized by Tregs to promote self-tolerance may be co-opted in the tumor microenvironment to suppress the anti-tumor immune response. Indeed, systemic depletion of Tregs in mice is sufficient to enable immune-mediated tumor regression (Teng et al., Cancer Research 70 (20): 7800-9, 2010). Thus, Tregs are thought to play a role in mediating peripheral tolerance to self-antigens, preventing autoimmune disease, and suppressing anti-tumor immune responses.

Tregs are found at high frequencies in the tumor tissue of various types of solid tumors, such as breast carcinoma, ovarian carcinoma, renal cell carcinoma (RCC), cervix carcinoma, prostate carcinoma, muscle invasive bladder carcinoma (MIBC), non-small cell lung carcinoma (NSCLC), hepatocellular carcinoma (HCC), pancreatic adenocarcinoma (PDAC), brain tumors, head and neck squamous cell carcinoma (HNSCC) and melanoma. Their high frequency among CD4+ T cells within tumor-infiltrating lymphocytes (TILs) or a high ratio of Foxp3+ Tregs to CD8+ cells is associated with a poor prognosis in the majority of solid tumors (for review, Tanaka, A. and Sakaguchi, S., Cell Res., 27:109-118, 2017).

Chemokines (chemoattractant cytokines) are comprised of a family of structurally and functionally related polypeptides with 8-10 kilodaltons. Chemokines are involved in diverse biological functions including regulation of immune cell proliferation, migration, activation, differentiation and homing. The biological activities of chemokines are mediated by a family of 7-transmembrane G-protein coupled receptors (GPCRs). CCR4, CCR8, CCR10, and CXCR3 are chemokine receptors responsible for Treg cell migration to the tumor microenvironment (TME) in response to CC and CXC chemokines: CCR4 is bound by CCL17 and CCL22; CCR8 is bound by CCL1; CCR10 is bound by CCL28; and CXCR3 is activated by CXCL9/10/11.

CCR8 (also previously called CY6, CKR-L1 or TER1) is a chemokine receptor that has recently been identified as a potential specific marker for tumor-infiltrating Tregs, as CCR8 expression is selectively upregulated in these Tregs in multiple cancers, including breast, colorectal, and lung (Wang L, et al., Nature Immunol 20:1220-30, 2019). These CCR8+ Tregs represent a highly activated and suppressive subpopulation of Tregs, and high abundance of CCR8⁺ Tregs in these tumor types is associated with poor prognosis (Id).

Tumor immunotherapy is based on the concept that the immune system is capable of recognizing tumors and eliminating malignant cells. Immunotherapy using agonistic, antagonistic, or blocking antibodies to co-stimulatory or co-inhibitory molecules (immune checkpoints) has been an area of extensive research and clinical evaluation. Immune checkpoint proteins include CTLA-4, PD-1, PD-L1, LAG-3, TIGIT and TIM-3 as well as several others (Sharpe et al., Nat Immunol, 8:239-45, 2007). Under normal physiological conditions, immune checkpoints are crucial for the maintenance of self-tolerance (that is, the prevention of autoimmunity) and protect tissues from damage when the immune system is responding to pathogenic infection. It is now also clear that tumors co-opt certain immune-checkpoint pathways as a major mechanism of immune resistance, particularly against T cells that are specific for tumor antigens (Pardoll D M., Nat Rev Cancer, 12:252-64, 2012). Inhibition of the PD-1 interaction with its dominant ligand PD-L1 mediates potent antitumor activity in preclinical models (U.S. Pat. Nos. 8,008,449 and 7,943,743), and the use of mAb inhibitors of the PD-1/PD-L1 interaction for treating cancer has become standard of care for many types of cancer (see, e.g., Topalian et al., Curr Opin Immunol., 24:207-212, 2012; Brahmer et al., N Engl J Med., 366 (26): 2455-65, 2012; Garon et al., N Engl J Med, 372:2018-2028, 2015; Philips et al., Int. Immunol., 27 (1): 39-46, 2015; Migden et al., N Engl J Med, 379:341-351, 2018). PD-1 expression has been found on tumor infiltrating T cells and PD-L1 expression has been found on tumor cells and myeloid cells within the tumor in many murine and human cancers, including human lung, ovarian and colon carcinoma and various myelomas, and anti-PD-1 and anti-PD-L1 antibodies developed by, e.g., Bristol-Myers Squibb (nivolumab), Merck (pembrolizumab), Regeneron (cemiplimab), Roche (atezolizumab), AstraZeneca (durvalumab) have been approved by the FDA to treat various cancer indications. The tolerability of PD-1-pathway blockers and their unique mechanism of action have made them ideal backbones for combination regimen development. Recent clinical data combining CTLA-4 and PD-1 blockade in melanoma patients showed an increased rate of objective tumor responses as compared to blocking either checkpoint alone, supporting the notion that combinatorial checkpoint blockade may result in increased clinical benefit (Wolchok et al., N Engl J Med, 366:2443-54, 2012). The combination of Yervoy and Opdivo has been approved for treatment of certain patients with melanoma, mesothelioma, non-small cell lung cancer, hepatocellular carcinoma, colorectal cancer, and rental cell carcinoma. Despite the positive rates of clinical responses observed, many more patients with advanced solid tumors are resistant or become resistant to immunotherapy (Rizvi et al., Cancer immunology, Science, 348 (6230): 124-128, 2015).

### DISCLOSURE OF THE INVENTION

In accordance with the present invention, there are provided isolated antibodies, and antigen-binding fragments thereof, that specifically bind C—C chemokine receptor type 8 (CCR8), and methods for treating a cancer in a subject comprising administering to the subject an anti-CCR8 Ab as monotherapy or in combination with an anticancer agent such as an immune checkpoint inhibitor. The present inventors propose that the anti-CCR8 mAbs provided herein could be utilized as safe and effective tumor infiltrating Treg antagonizing and depleting agents that also spare T effector cells (Teffs) for optimal anti-tumor responses. The CCR8 mAbs provide a safer alternative to other Treg depleting strategies as only the tumor infiltrating Treg express the high levels of CCR8 required for depletion via ADCC, thus sparing Treg in peripheral tissues that function to maintain immune homeostasis. Depletion of Treg via anti-CTLA-4, anti-CD25, and other Treg surface markers may result in depletion of both tumor and peripheral Treg which can lead to severe autoimmune side effects, as well as potential depletion of beneficial anti-tumor conventional T cells expressing the shared target antigen.

In various embodiments, the antibody or antigen-binding fragment is selected from a human antibody, a humanized antibody, chimeric antibody, a monoclonal antibody, a polyclonal antibody, a recombinant antibody, a single chain antibody, a diabody, a triabody, a tetrabody, a Fab fragment, a Fab′ fragment, a Fab₂ fragment, a F(ab)′₂ fragment, a domain antibody, an Afucosylated antibody, an IgD antibody, an IgE antibody, an IgM antibody, an IgG1 antibody, an IgG2 antibody, an IgG3 antibody, an IgG4 antibody, an IgG1 antibody having at least one mutation that enhances ADCC/FcR affinity, or an IgG4 antibody having at least one mutation in the hinge region that alleviates a tendency to form intra H-chain disulfide bonds. In various embodiments, the antibody is a chimeric antibody. In various embodiments, the antibody is a humanized antibody. In various embodiments, the antibody is a fully human antibody. In various embodiments, isolated antibodies, and antigen-binding fragments thereof, that have a high affinity for the human CCR8 of SEQ ID NO: 1 are provided.

In various embodiments, the antibody or antigen-binding fragment binds to CCR8 protein with a dissociation constant (K_{D}) of at least about 1×10⁻⁶ M, at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M.

In various embodiments, the isolated humanized or human monoclonal antibody or antigen-binding fragment thereof of the present invention binds to human CCR8 and comprises: (a) a heavy chain CDR1 sequence selected from the group of amino acid sequences defined by SEQ ID NOS: 3, 7, and 9; (b) a heavy chain CDR2 sequence selected from the group of amino acid sequences defined by SEQ ID NOS: 4, 8, 10 and 35-38; (c) a heavy chain CDR3 sequence selected from the group of amino acid sequences defined by SEQ ID NOS: 5, 6, 11 and 39-45; (d) a light chain CDR1 sequence selected from the group of amino acid sequences defined by SEQ ID NOS: 12, 15 and 46-47; (e) a light chain CDR2 sequence selected from the group of amino acid sequences defined by SEQ ID NOS: 13, 16 and 48-49; and (f) a light chain CDR3 sequence selected from the group of amino acid sequences defined by SEQ ID NOS: 14, 17 and 50-52.

In various embodiments, the isolated humanized or human monoclonal antibody or antigen-binding fragment thereof of the present invention binds to human CCR8 and comprises: (1) a heavy chain CDR1 sequence of SEQ ID NO: 3; a heavy chain CDR2 sequence of SEQ ID NO: 4; a heavy chain CDR3 sequence of SEQ ID NO: 5; a light chain CDR1 sequence of SEQ ID NO: 12; a light chain CDR2 sequence of SEQ ID NO: 13; and a light chain CDR3 sequence of SEQ ID NO: 14; or (2) a heavy chain CDR1 sequence of SEQ ID NO: 3; a heavy chain CDR2 sequence of SEQ ID NO: 4; a heavy chain CDR3 sequence of SEQ ID NO: 6; a light chain CDR1 sequence of SEQ ID NO: 12; a light chain CDR2 sequence of SEQ ID NO: 13; and a light chain CDR3 sequence of SEQ ID NO: 14; or (3) a heavy chain CDR1 sequence of SEQ ID NO: 7; a heavy chain CDR2 sequence of SEQ ID NO: 8; a heavy chain CDR3 sequence of SEQ ID NO: 6; a light chain CDR1 sequence of SEQ ID NO: 12; a light chain CDR2 sequence of SEQ ID NO: 13; and a light chain CDR3 sequence of SEQ ID NO: 14; or (4) a heavy chain CDR1 sequence of SEQ ID NO: 9; a heavy chain CDR2 sequence of SEQ ID NO: 10; a heavy chain CDR3 sequence of SEQ ID NO: 11; a light chain CDR1 sequence of SEQ ID NO: 15; a light chain CDR2 sequence of SEQ ID NO: 16; and a light chain CDR3 sequence of SEQ ID NO: 17; or (5) a heavy chain CDR1 sequence of SEQ ID NO: 7; a heavy chain CDR2 sequence of SEQ ID NO: 35; a heavy chain CDR3 sequence of SEQ ID NO: 39; a light chain CDR1 sequence of SEQ ID NO: 12; a light chain CDR2 sequence of SEQ ID NO: 13; and a light chain CDR3 sequence of SEQ ID NO: 14; or (6) a heavy chain CDR1 sequence of SEQ ID NO: 3; a heavy chain CDR2 sequence of SEQ ID NO: 35; a heavy chain CDR3 sequence of SEQ ID NO: 40; a light chain CDR1 sequence of SEQ ID NO: 12; a light chain CDR2 sequence of SEQ ID NO: 13; and a light chain CDR3 sequence of SEQ ID NO: 14; or (7) a heavy chain CDR1 sequence of SEQ ID NO: 3; a heavy chain CDR2 sequence of SEQ ID NO: 35; a heavy chain CDR3 sequence of SEQ ID NO: 40; a light chain CDR1 sequence of SEQ ID NO: 12; a light chain CDR2 sequence of SEQ ID NO: 13; and a light chain CDR3 sequence of SEQ ID NO: 14; or (8) a heavy chain CDR1 sequence of SEQ ID NO: 7; a heavy chain CDR2 sequence of SEQ ID NO: 4; a heavy chain CDR3 sequence of SEQ ID NO: 41; a light chain CDR1 sequence of SEQ ID NO: 12; a light chain CDR2 sequence of SEQ ID NO: 13; and a light chain CDR3 sequence of SEQ ID NO: 14; or (9) a heavy chain CDR1 sequence of SEQ ID NO: 7; a heavy chain CDR2 sequence of SEQ ID NO: 35; a heavy chain CDR3 sequence of SEQ ID NO: 42; a light chain CDR1 sequence of SEQ ID NO: 12; a light chain CDR2 sequence of SEQ ID NO: 13; and a light chain CDR3 sequence of SEQ ID NO: 14; or (10) a heavy chain CDR1 sequence of SEQ ID NO: 7; a heavy chain CDR2 sequence of SEQ ID NO: 35; a heavy chain CDR3 sequence of SEQ ID NO: 43; a light chain CDR1 sequence of SEQ ID NO: 12; a light chain CDR2 sequence of SEQ ID NO: 13; and a light chain CDR3 sequence of SEQ ID NO: 14; or (11) a heavy chain CDR1 sequence of SEQ ID NO: 7; a heavy chain CDR2 sequence of SEQ ID NO: 35; a heavy chain CDR3 sequence of SEQ ID NO: 43; a light chain CDR1 sequence of SEQ ID NO: 12; a light chain CDR2 sequence of SEQ ID NO: 13; and a light chain CDR3 sequence of SEQ ID NO: 14; or (12) a heavy chain CDR1 sequence of SEQ ID NO: 7; a heavy chain CDR2 sequence of SEQ ID NO: 4; a heavy chain CDR3 sequence of SEQ ID NO: 44; a light chain CDR1 sequence of SEQ ID NO: 46; a light chain CDR2 sequence of SEQ ID NO: 48; and a light chain CDR3 sequence of SEQ ID NO: 50; or (13) a heavy chain CDR1 sequence of SEQ ID NO: 3; a heavy chain CDR2 sequence of SEQ ID NO: 4; a heavy chain CDR3 sequence of SEQ ID NO: 44; a light chain CDR1 sequence of SEQ ID NO: 46; a light chain CDR2 sequence of SEQ ID NO: 48; and a light chain CDR3 sequence of SEQ ID NO: 50; or (14) a heavy chain CDR1 sequence of SEQ ID NO: 7; a heavy chain CDR2 sequence of SEQ ID NO: 4; a heavy chain CDR3 sequence of SEQ ID NO: 45; a light chain CDR1 sequence of SEQ ID NO: 47; a light chain CDR2 sequence of SEQ ID NO: 13; and a light chain CDR3 sequence of SEQ ID NO: 14; or (15) a heavy chain CDR1 sequence of SEQ ID NO: 3; a heavy chain CDR2 sequence of SEQ ID NO: 38; a heavy chain CDR3 sequence of SEQ ID NO: 44; a light chain CDR1 sequence of SEQ ID NO: 46; a light chain CDR2 sequence of SEQ ID NO: 48; and a light chain CDR3 sequence of SEQ ID NO: 51; or (16) a heavy chain CDR1 sequence of SEQ ID NO: 7; a heavy chain CDR2 sequence of SEQ ID NO: 35; a heavy chain CDR3 sequence of SEQ ID NO: 42; a light chain CDR1 sequence of SEQ ID NO: 12; a light chain CDR2 sequence of SEQ ID NO: 13; and a light chain CDR3 sequence of SEQ ID NO: 14; or (17) a heavy chain CDR1 sequence of SEQ ID NO: 7; a heavy chain CDR2 sequence of SEQ ID NO: 36; a heavy chain CDR3 sequence of SEQ ID NO: 45; a light chain CDR1 sequence of SEQ ID NO: 12; a light chain CDR2 sequence of SEQ ID NO: 49; and a light chain CDR3 sequence of SEQ ID NO: 14; or (18) a heavy chain CDR1 sequence of SEQ ID NO: 7; a heavy chain CDR2 sequence of SEQ ID NO: 37; a heavy chain CDR3 sequence of SEQ ID NO: 42; a light chain CDR1 sequence of SEQ ID NO: 12; a light chain CDR2 sequence of SEQ ID NO: 13; and a light chain CDR3 sequence of SEQ ID NO: 14; or (19) a heavy chain CDR1 sequence of SEQ ID NO: 3; a heavy chain CDR2 sequence of SEQ ID NO: 38; a heavy chain CDR3 sequence of SEQ ID NO: 44; a light chain CDR1 sequence of SEQ ID NO: 46; a light chain CDR2 sequence of SEQ ID NO: 48; and a light chain CDR3 sequence of SEQ ID NO: 52.

In various embodiments, an isolated antibody or antigen-binding fragment thereof of the present invention binds to human CCR8 and comprises either: (a) a heavy chain CDR1 sequence selected from the group of amino acid sequences defined by SEQ ID NOS: 3, 7, and 9; (b) a heavy chain CDR2 sequence selected from the group of amino acid sequences defined by SEQ ID NOS: 4, 8, 10 and 35-38; (c) a heavy chain CDR3 sequence selected from the group of amino acid sequences defined by SEQ ID NOS: 5, 6, 11 and 39-45; (d) a light chain CDR1 sequence selected from the group of amino acid sequences defined by SEQ ID NOS: 12, 15 and 46-47; (e) a light chain CDR2 sequence selected from the group of amino acid sequences defined by SEQ ID NOS: 13, 16 and 48-49; and (f) a light chain CDR3 sequence selected from the group of amino acid sequences defined by SEQ ID NOS: 14, 17 and 50-52; and (g) a set of four variable region framework regions from a human immunoglobulin (IgG). In various embodiments, the antibody can optionally include a hinge region. In various embodiments, the framework regions are chosen from human germline exon X_{H}, J_{H}, V_{K} and J_{K} sequences. In various embodiments, the antibody is a fully humanized antibody. In various embodiments, the antibody is a fully human antibody.

In various embodiments, an isolated antibody or antigen-binding fragment thereof of the present invention binds to human CCR8 and comprises the heavy chain variable region having the amino acid sequence set forth in SEQ ID NO: 18 and the light chain variable region having the amino acid sequence set forth in SEQ ID NO: 19; or the heavy chain variable region having the amino acid sequence set forth in SEQ ID NO: 20 and the light chain variable region having the amino acid sequence set forth in SEQ ID NO: 21; or the heavy chain variable region having the amino acid sequence set forth in SEQ ID NO: 22 and the light chain variable region having the amino acid sequence set forth in SEQ ID NO: 23; or the heavy chain variable region having the amino acid sequence set forth in SEQ ID NO: 24 and the light chain variable region having the amino acid sequence set forth in SEQ ID NO: 25; or the heavy chain variable region having the amino acid sequence set forth in SEQ ID NO: 53 and the light chain variable region having the amino acid sequence set forth in SEQ ID NO: 68; or the heavy chain variable region having the amino acid sequence set forth in SEQ ID NO: 54 and the light chain variable region having the amino acid sequence set forth in SEQ ID NO: 68; or the heavy chain variable region having the amino acid sequence set forth in SEQ ID NO: 55 and the light chain variable region having the amino acid sequence set forth in SEQ ID NO: 68; or the heavy chain variable region having the amino acid sequence set forth in SEQ ID NO: 56 and the light chain variable region having the amino acid sequence set forth in SEQ ID NO: 69; or the heavy chain variable region having the amino acid sequence set forth in SEQ ID NO: 57 and the light chain variable region having the amino acid sequence set forth in SEQ ID NO: 69; or the heavy chain variable region having the amino acid sequence set forth in SEQ ID NO: 58 and the light chain variable region having the amino acid sequence set forth in SEQ ID NO: 70; or the heavy chain variable region having the amino acid sequence set forth in SEQ ID NO: 59 and the light chain variable region having the amino acid sequence set forth in SEQ ID NO: 70; or the heavy chain variable region having the amino acid sequence set forth in SEQ ID NO: 60 and the light chain variable region having the amino acid sequence set forth in SEQ ID NO: 71; or the heavy chain variable region having the amino acid sequence set forth in SEQ ID NO: 61 and the light chain variable region having the amino acid sequence set forth in SEQ ID NO: 71; or the heavy chain variable region having the amino acid sequence set forth in SEQ ID NO: 62 and the light chain variable region having the amino acid sequence set forth in SEQ ID NO: 72; or the heavy chain variable region having the amino acid sequence set forth in SEQ ID NO: 63 and the light chain variable region having the amino acid sequence set forth in SEQ ID NO: 73; or the heavy chain variable region having the amino acid sequence set forth in SEQ ID NO: 64 and the light chain variable region having the amino acid sequence set forth in SEQ ID NO: 74; or the heavy chain variable region having the amino acid sequence set forth in SEQ ID NO: 65 and the light chain variable region having the amino acid sequence set forth in SEQ ID NO: 75; or the heavy chain variable region having the amino acid sequence set forth in SEQ ID NO: 66 and the light chain variable region having the amino acid sequence set forth in SEQ ID NO: 76; or the heavy chain variable region having the amino acid sequence set forth in SEQ ID NO: 67 and the light chain variable region having the amino acid sequence set forth in SEQ ID NO: 77.

In various embodiments, an isolated antibody or antigen-binding fragment thereof of the present invention binds to human CCR8 is an isolated chimeric antibody or antigen-binding fragment thereof and comprises: (1) the heavy chain sequence of SEQ ID NO: 26 and the light chain sequence of SEQ ID NO: 27; or (2) the heavy chain sequence of SEQ ID NO: 28 and the light chain sequence of SEQ ID NO: 29; or (3) the heavy chain sequence of SEQ ID NO: 30 and the light chain sequence of SEQ ID NO: 31; or (4) the heavy chain sequence of SEQ ID NO: 78 and the light chain sequence of SEQ ID NO: 79; or (5) the heavy chain sequence of SEQ ID NO: 80 and the light chain sequence of SEQ ID NO: 81; or (6) the heavy chain sequence of SEQ ID NO: 82 and the light chain sequence of SEQ ID NO: 83; or (7) the heavy chain sequence of SEQ ID NO: 84 and the light chain sequence of SEQ ID NO: 85.

In various embodiments, an isolated humanized antibody or antigen-binding fragment thereof of the present invention binds to human CCR8 and comprises: (a) a heavy chain sequence selected from the group of amino acid sequences defined by SEQ ID NOS: 86, 88 and 90; and (b) a light chain sequence selected from the group of amino acid sequences defined by SEQ ID NOS: 87, 89 and 91-92.

In another aspect, the present invention relates to a pharmaceutical composition comprising an isolated antibody or antigen-binding fragment of the present invention in admixture with a pharmaceutically acceptable carrier. In various embodiments, the pharmaceutical composition comprises an isolated human antibody in admixture with a pharmaceutically acceptable carrier. In various embodiments, the pharmaceutical composition is formulated for administration via a route selected from the group consisting of subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or via infusions.

In another aspect, the present invention relates to methods of treating a subject suffering from a CCR8-associated disorder, comprising administering to said subject a therapeutically effective amount of an antibody or antigen-binding fragment thereof of the present invention. In various embodiments, the subject is a human subject. In various embodiments, the CCR8-associated disorder is a cancer. In various embodiments, the subject previously responded to treatment with an anti-cancer therapy, but, upon cessation of therapy, suffered relapse (hereinafter “a recurrent cancer”). In various embodiments, the subject has resistant or refractory cancer. In various embodiments, the cancerous cells are immunogenic tumors (e.g., those tumors for which vaccination using the tumor itself can lead to immunity to tumor challenge).

In various embodiments, a method for treating a subject afflicted with a cancer comprises administering to the subject a therapeutically effective amount of any one of the Treg-depleting anti-CCR8 Abs, e.g., mAbs, immunoconjugates or bispecific molecules disclosed herein, or a pharmaceutical composition comprising any one of said Abs, e.g., anti-CCR8 mAbs, immunoconjugates or bispecific molecules, such that the subject is treated.

In another aspect, the present invention relates to combination therapies designed to treat a cancer in a subject. In various embodiments, a method for inhibiting growth of tumor cells in a subject comprises administering to the subject a therapeutically effective amount of: (a) any one of the Treg-depleting anti-CCR8 Abs, immunoconjugates or bispecific molecules disclosed herein, or a pharmaceutical composition comprising any one of said anti-CCR8 Abs, immunoconjugates or bispecific molecules; and (b) an additional therapy for treating cancer. In various embodiments, the additional therapeutic therapy is a therapeutic agent that is a compound that reduces inhibition, or increases stimulation, of the immune system, such that growth of tumor cells in the subject is inhibited. In various embodiments, the additional therapy is selected from the group consisting of immunotherapy, chemotherapy, small molecule kinase inhibitor targeted therapy, surgery, radiation therapy, and stem cell transplantation, wherein the combination therapy provides increased cell killing of tumor cells, i.e., a synergy exists between the isolated antibody or antigen-binding fragment and the additional therapies when co-administered.

In another aspect, the present invention relates to methods for enhancing the immune response to cancerous cells in a subject, comprising administering to the subject a therapeutically effective amount (either as monotherapy or in a combination therapy regimen) of an isolated antibody or antigen-binding fragment of the present invention. In various embodiments, the present invention provides for a method of treating cancerous cells in a subject, comprising administering to said subject a therapeutically effective amount (either as monotherapy or in a combination therapy regimen) of an antibody or antigen-binding fragment thereof of the present invention. In various embodiments, the cancerous cell is selected from the group consisting of ovarian cancer, lung cancer, breast cancer, gastric cancer, prostate cancer, colorectal cancer, renal cell cancer, liver cancer, pancreatic cancer, glioblastoma, melanoma and sarcoma.

In another aspect, an isolated immunoconjugate or fusion protein comprising an antibody or antigen-binding fragment conjugated to, linked to (or otherwise stably associated with) an effector molecule is provided. In various embodiments, the effector molecule is an immunotoxin, cytokine, chemokine, therapeutic agent, or chemotherapeutic agent.

In another aspect, the present invention features bispecific molecules comprising an anti-CCR8 antibody, or antigen-binding fragment thereof, of the invention. In various embodiments, an antibody of the invention, or antigen-binding fragment thereof, can be derivatized or linked to another functional molecule, e.g., another peptide or protein (e.g., another antibody or ligand for a receptor) to generate a bispecific molecule that binds to at least two different binding sites or target molecules. In various embodiments, the antibody of the invention may in fact be derivatized or linked to more than one other functional molecule to generate multispecific molecules that bind to more than two different binding sites and/or target molecules. In various embodiments, the bispecific molecule is an anti-CCR8 antibody, or antigen-binding fragment thereof, of the invention linked to a functional molecule that binds CTLA-4. In various embodiments, the bispecific molecule is a bispecific antibody comprising a heavy chain having the amino acid sequence of SEQ ID NO: 93 and a light chain having the amino acid sequence of SEQ ID NO: 91. In various embodiments, the bispecific molecule is a bispecific antibody comprising a heavy chain having the amino acid sequence of SEQ ID NO: 93 and a light chain having the amino acid sequence of SEQ ID NO: 92.

In another aspect, the antibodies or antigen-binding fragments disclosed herein may be covalently linked to (or otherwise stably associated with) an additional functional moiety, such as a label or a moiety that confers desirable pharmacokinetic properties. In various embodiments, the label is selected from the group consisting of a fluorescent label, a radioactive label, and a label having a distinctive nuclear magnetic resonance signature.

In another aspect, the present invention provides a method for detecting in vitro or in vivo the presence of human CCR8 peptide in a sample, e.g., for diagnosing a human CCR8-associated disorder.

### BRIEF DESCRIPTIONS OF THE DRAWINGS

FIG. 1 is a line graph depicting the dose response of anti-CCR8 antibodies in blocking CCR8-mediated calcium flux induced by hCCL1. Reference Ab #1 is a humanized anti-hCCR8 antibody that has been described in the literature.

FIG. 2 is a line graph depicting the dose response of anti-CCR8 antibodies in blocking CCR8-mediated calcium flux induced by hCCL1. Reference Abs #1 and #2 are humanized anti-hCCR8 antibodies that have been described in the literature.

FIG. 3 is a set of diagrams showing that bispecific antibodies FP578-01 and FP578-02 can bind to CTLA-4 while they are concurrently bound to CCR8.

### MODE(S) OF CARRYING OUT THE INVENTION

The present invention relates to antigen binding proteins such as antibodies, or antigen-binding fragments thereof that specifically bind to human CCR8. In one aspect, there are provided isolated antibodies, and antigen-binding fragments thereof, that specifically bind CCR8, have a high affinity for CCR8, function to inhibit CCR8, are less immunogenic compared to their unmodified parent antibodies in a given species (e.g., a human), and can be used to treat human disorders mediated by CCR8. Also provided are nucleic acid molecules, and derivatives and fragments thereof, comprising a sequence of polynucleotides that encode all or a portion of a polypeptide that binds to CCR8, such as a nucleic acid encoding all or part of an anti-CCR8 antibody, antibody fragment, or antibody derivative. Also provided are vectors and plasmids comprising such nucleic acids, and cells or cell lines comprising such nucleic acids and/or vectors and plasmids. Also provided are methods of making, identifying, or isolating antigen binding proteins that bind to human CCR8, such as anti-CCR8 antibodies, methods of determining whether an antigen binding protein binds to CCR8, methods of making compositions, such as pharmaceutical compositions, comprising an antigen binding protein that binds to human CCR8, and methods for administering an antibody, or antigen-binding fragment thereof that binds CCR8 to a subject, for example, methods for treating a condition mediated by CCR8.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

The terms “polypeptide”, “peptide” and “protein” are used interchangeably herein to refer to a polymer of amino acid residues. In various embodiments, “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond.

Polypeptides of the disclosure include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties.

An amino acid “substitution” as used herein refers to the replacement in a polypeptide of one amino acid at a particular position in a parent polypeptide sequence with a different amino acid. Amino acid substitutions can be generated using genetic or chemical methods well known in the art. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” refers to the substitution in a polypeptide of an amino acid with a functionally similar amino acid. The following six groups each contain amino acids that are conservative substitutions for one another:
1) Alanine (A), Serine(S), and Threonine (T)
2) Aspartic acid (D) and Glutamic acid (E)
3) Asparagine (N) and Glutamine (Q)
4) Arginine (R) and Lysine (K)
5) Isoleucine (I), Leucine (L), Methionine (M), and Valine (V)
6) Phenylalanine (F), Tyrosine (Y), and Tryptophan (W)

A “non-conservative amino acid substitution” refers to the substitution of a member of one of these classes for a member from another class. In making such changes, according to various embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in various embodiments, the substitution of amino acids whose hydropathic indices are within ±2 is included. In various embodiments, those that are within ±1 are included, and in various embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In various embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in various embodiments, the substitution of amino acids whose hydrophilicity values are within ±2 is included, in various embodiments, those that are within ±1 are included, and in various embodiments, those within ±0.5 are included.

Exemplary amino acid substitutions are set forth in Table 1.

TABLE 1

| Original | | |
|---|---|---|
| Residues | Exemplary Substitutions | Preferred Substitutions |
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable variants of polypeptides as set forth herein using well-known techniques. In various embodiments, one skilled in the art may identify suitable areas of the molecule that may be changed without destroying activity by targeting regions not believed to be important for activity. In other embodiments, the skilled artisan can identify residues and portions of the molecules that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a polypeptide that correspond to amino acid residues important for activity or structure in similar polypeptides. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three-dimensional structure. In various embodiments, one skilled in the art may choose to not make radical changes to amino acid residues predicted to be on the surface of the polypeptide, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

The term “polypeptide fragment” and “truncated polypeptide” as used herein refers to a polypeptide that has an amino-terminal and/or carboxy-terminal deletion as compared to a corresponding full-length protein. In various embodiments, fragments can be, e.g., at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

The terms “polypeptide variant”, “hybrid polypeptide” and “polypeptide mutant” as used herein refers to a polypeptide that comprises an amino acid sequence wherein one or more amino acid residues are inserted into, deleted from and/or substituted into the amino acid sequence relative to another polypeptide sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Hybrids of the present disclosure include fusion proteins.

As described herein, a single mutation will be identified by the particular amino acid substitution at a specific amino acid position within the sequence of a wild-type CCR8. For example, for the human CCR8 provided as SEQ ID NO: 1, a mutation comprising a serine substituted for the full-length wild-type threonine at amino acid 10 is identified as T10S.

A “derivative” of a polypeptide is a polypeptide that has been chemically modified, e.g., conjugation to another chemical moiety such as, for example, polyethylene glycol, albumin (e.g., human serum albumin), phosphorylation, and glycosylation.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm and means that a given sequence is at least 80% identical to another length of another sequence. In various embodiments, the % identity is selected from, e.g., at least 60%, at least 65%, at least 70%, at least 75%, at least 80%, at least 85%, at least 90%, at least 95%, or at least 99% or more sequence identity to a given sequence. In various embodiments, the % identity is in the range of, e.g., about 60% to about 70%, about 70% to about 80%, about 80% to about 85%, about 85% to about 90%, about 90% to about 95%, or about 95% to about 99%.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In various embodiments, the % homology is selected from, e.g., at least 60%, at least 65%, at least 70%, at least 75%, at least 80%, at least 85%, at least 90%, at least 95%, or at least 99% or more sequence homology to a given sequence. In various embodiments, the % homology is in the range of, e.g., about 60% to about 70%, about 70% to about 80%, about 80% to about 85%, about 85% to about 90%, about 90% to about 95%, or about 95% to about 99%.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., J. Mol. Biol. 215:403-10, 1990 (with special reference to the published default setting, i.e., parameters w=4, t=17) and Altschul et al., Nucleic Acids Res., 25:3389-3402, 1997. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix.

In addition to calculating percent sequence identity, the BLAST algorithm also performs a statistical analysis of the similarity between two sequences (see, e.g., Karlin & Altschul, Proc. Natl. Acad. Sci. USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is, e.g., less than about 0.1, less than about 0.01, or less than about 0.001.

The term “modification” as used herein refers to any manipulation of the peptide backbone (e.g., amino acid sequence) or the post-translational modifications (e.g., glycosylation) of a polypeptide.

The term “therapeutic protein” refers to proteins, polypeptides, antibodies, peptides or fragments or variants thereof, having one or more therapeutic and/or biological activities. Therapeutic proteins encompassed by the invention include but are not limited to, proteins, polypeptides, peptides, antibodies, and biologics (the terms peptides, proteins, and polypeptides are used interchangeably herein). It is specifically contemplated that the term “therapeutic protein” encompasses the fusion molecules of the present invention.

The term “fusion protein” as used herein refers to a fusion polypeptide molecule comprising two or more genes that originally coded for separate proteins, wherein the components of the fusion protein are linked to each other by peptide-bonds, either directly or through peptide linkers. The term “fused” as used herein refers to components that are linked by peptide bonds, either directly or via one or more peptide linkers.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc.

The term “peptide linker” as used herein refers to a peptide comprising one or more amino acids, typically about 2-20 amino acids. Peptide linkers are known in the art or are described herein. Suitable, non-immunogenic linker peptides include, for example, (G₄S)ₙ, (SG₄)ₙ or G₄(SG₄)ₙ peptide linkers. “n” is generally a number between 1 and 10, typically between 2 and 4.

The term “tumor associated antigen” (TAA) refers to, e.g., cell surface antigens that are selectively expressed by cancer cells or over-expressed in cancer cells relative to most normal cells. The terms “TAA variant” and “TAA mutant” as used herein refers to a TAA that comprises an amino acid sequence wherein one or more amino acid residues are inserted into, deleted from and/or substituted into the amino acid sequence relative to another TAA sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length.

The term “antibody” is used herein to refer to a protein comprising one or more polypeptides substantially or partially encoded by immunoglobulin genes or fragments of immunoglobulin genes and having specificity to a tumor antigen or specificity to a molecule overexpressed in a pathological state. The recognized immunoglobulin genes include the kappa, lambda, alpha, gamma, delta, epsilon and mu constant region genes, as well as subtypes of these genes and myriad of immunoglobulin variable region genes. Light chains (LC) are classified as either kappa or lambda. Heavy chains (HC) are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition.

In a full-length antibody, each heavy chain is comprised of a heavy chain variable region (abbreviated herein as HCVR or VH) and a heavy chain constant region. The heavy chain constant region is comprised of three domains, CH1, CH2 and CH3 (and in some instances, CH4). Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The extent of the framework region and CDRs has been defined. The sequences of the framework regions of different light or heavy chains are relatively conserved within a species, such as humans. The framework region of an antibody, that is the combined framework regions of the constituent light and heavy chains, serves to position and align the CDRs in three-dimensional space. Immunoglobulin molecules can be of any type (e.g., IgG, IgE, IgM, IgD, IgA and IgY), class (e.g., IgG1, IgG2, IgG 3, IgG4, IgA1 and IgA2) or subclass.

The CDRs are primarily responsible for binding to an epitope of an antigen. The CDRs of each chain are typically referred to as CDR1, CDR2, CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

The Kabat definition is a standard for numbering the residues in an antibody and is typically used to identify CDR regions. The Kabat database is now maintained online and CDR sequences can be determined, for example, see IMGT/V-QUEST programme version: 3.2.18 Mar. 29, 2011, available on the internet and Brochet, X. et al., Nucl. Acids Res. 36, W503-508, 2008). The Chothia definition is similar to the Kabat definition, but the Chothia definition takes into account positions of certain structural loop regions. See, e.g., Chothia et al., J. Mol. Biol., 196:901-17, 1986; Chothia et al., Nature, 342:877-83, 1989. The AbM definition uses an integrated suite of computer programs produced by Oxford Molecular Group that model antibody structure. See, e.g., Martin et al., Proc. Natl. Acad. Sci. USA, 86:9268-9272, 1989; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198, 1999. The contact definition is based on an analysis of the available complex crystal structures. See, e.g., MacCallum et al., J. Mol. Biol., 5:732-45, 1996.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. The Fc portion of an antibody mediates several important effector functions e.g. cytokine induction, ADCC, phagocytosis, complement dependent cytotoxicity (CDC) and half-life/clearance rate of antibody and antigen-antibody complexes (e.g., the neonatal FcR (FcRn) binds to the Fc region of IgG at acidic pH in the endosome and protects IgG from degradation, thereby contributing to the long serum half-life of IgG). Replacements of amino acid residues in the Fc portion to alter antibody effector function are known in the art (see, e.g., Winter et al., U.S. Pat. Nos. 5,648,260 and 5,624,821).

Antibodies exist as intact immunoglobulins or as a number of well characterized fragments. Such fragments include Fab fragments, Fab′ fragments, Fab₂, F(ab)′₂ fragments, single chain Fv proteins (“scFv”) and disulfide stabilized Fv proteins (“dsFv”), that bind to the target antigen. A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. While various antibody fragments are defined in terms of the digestion of an intact antibody, one of skill will appreciate that such fragments may be synthesized de novo either chemically or by utilizing recombinant DNA methodology. Thus, as used herein, the term antibody encompasses e.g., monoclonal antibodies (including full-length monoclonal antibodies), polyclonal antibodies, multispecific antibodies (e.g., bispecific antibodies) formed from at least two intact antibodies, human antibodies, humanized antibodies, camelised antibodies, chimeric antibodies, single-chain Fvs (scFv), single-chain antibodies, single domain antibodies, domain antibodies, Fab fragments, F(ab′)₂ fragments, antibody fragments that exhibit the desired biological activity, disulfide-linked Fvs (sdFv), intrabodies, and epitope-binding fragments or antigen binding fragments of any of the above.

Papain digestion of antibodies produces two identical antigen-binding fragments, called “Fab” fragments, each with a single antigen-binding site. A “Fab fragment” comprises one light chain and the CH1 and variable regions of one heavy chain. The heavy chain of a Fab molecule cannot form a disulfide bond with another heavy chain molecule. A “Fab′ fragment” comprises one light chain and a portion of one heavy chain that contains the VH domain and the CH1 domain and also the region between the CH1 and CH2 domains, such that an interchain disulfide bond can be formed between the two heavy chains of two Fab′ fragments to form an F(ab′)₂ molecule.

Pepsin treatment of an antibody yields an F(ab′)₂ fragment that has two antigen-combining sites and is still capable of cross-linking antigen. A “F(ab′)₂ fragment” contains two light chains and two heavy chains containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains but lacks the constant regions.

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649, U.S. Pat. Nos. 4,946,778 and 5,260,203, the disclosures of which are incorporated by reference.

The terms “an antigen-binding fragment” and “antigen-binding protein” as used herein means any protein that binds a specified target antigen. “Antigen-binding fragment” includes but is not limited to antibodies and binding parts thereof, such as immunologically functional fragments. An exemplary antigen-binding fragment of an antibody is the heavy chain and/or light chain CDR(s), or the heavy and/or light chain variable region.

The term “immunologically functional fragment” (or simply “fragment”) of an antibody or immunoglobulin chain (heavy or light chain) antigen binding protein, as used herein, is a species of antigen binding protein comprising a portion (regardless of how that portion is obtained or synthesized) of an antibody that lacks at least some of the amino acids present in a full-length chain but which is still capable of specifically binding to an antigen. Such fragments are biologically active in that they bind to the target antigen and can compete with other antigen binding proteins, including intact antibodies, for binding to a given epitope. In some embodiments, the fragments are neutralizing fragments. In one aspect, such a fragment will retain at least one CDR present in the full-length light or heavy chain, and in some embodiments will comprise a single heavy chain and/or light chain or portion thereof. These biologically active fragments can be produced by recombinant DNA techniques or can be produced by enzymatic or chemical cleavage of antigen binding proteins, including intact antibodies. Immunologically functional immunoglobulin fragments include, but are not limited to, Fab, a diabody, Fab′, F(ab′)₂, Fv, domain antibodies and single-chain antibodies, and can be derived from any mammalian source, including but not limited to human, mouse, rat, camelid or rabbit. It is further contemplated that a functional portion of the antigen binding proteins disclosed herein, for example, one or more CDRs, could be covalently bound to a second protein or to a small molecule to create a therapeutic agent directed to a particular target in the body, possessing bifunctional therapeutic properties, or having a prolonged serum half-life.

Diabodies are bivalent antibodies comprising two polypeptide chains, wherein each polypeptide chain comprises VH and VL regions joined by a linker that is too short to allow for pairing between two regions on the same chain, thus allowing each region to pair with a complementary region on another polypeptide chain (see, e.g., Holliger et al., Proc. Natl. Acad. Sci. USA, 90:6444-48, 1993; and Poljak et al., Structure, 2:1121-23, 1994). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

Bispecific antibodies or fragments can be of several configurations. For example, bispecific antibodies may resemble single antibodies (or antibody fragments) but have two different antigen binding sites (variable regions). In various embodiments bispecific antibodies can be produced by chemical techniques (Kranz et al., Proc. Natl. Acad. Sci. USA, 78:5807, 1981; by “polydoma” techniques (see, e.g., U.S. Pat. No. 4,474,893); or by recombinant DNA techniques. In various embodiments bispecific antibodies of the present disclosure can have binding specificities for at least two different epitopes at least one of which is a tumor associate antigen. In various embodiments the antibodies and fragments can also be heteroantibodies. Heteroantibodies are two or more antibodies, or antibody binding fragments (e.g., Fab) linked together, each antibody or fragment having a different specificity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method.

The term “chimeric antibody” as used herein refers to an antibody which has framework residues from one species, such as human, and CDRs (which generally confer antigen binding) from another species, such as a murine antibody that specifically binds targeted antigen.

The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “humanized antibody” as used herein refers to an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. In various embodiments, the framework regions are chosen from human germline exon X_{H}, J_{H}, V_{K} and J_{K} sequences. For example, acceptor sequences for humanization of FR of a V_{H} domain can be chosen from genuine V_{H} exons V_{H} 1-18 (Matsuda et al., Nature Genetics 3:88-94, 1993) or V_{H}1-2 (Shin et al., EMBO J. 10:3641-3645, 1991) and for the hinge region (J_{H}), exon J_{H}-6 (Mattila et al., Eur. J. Immunol. 25:2578-2582, 1995). In other examples, germline V_{K} exon B3 (Cox et al., Eur. J. Immunol. 24:827-836, 1994) and J_{K} exon J_{K}-1 (Hieter et al., J. Biol. Chem. 257:1516-1522, 1982) can be chosen as acceptor sequences for V_{L} domain humanization.

The term “recombinant human antibody”, as used herein, is intended to include all human antibodies that are prepared, expressed, created or isolated by recombinant means, such as antibodies expressed using a recombinant expression vector transfected into a host cell; antibodies isolated from a recombinant, combinatorial human antibody library; antibodies isolated from an animal (e.g., a mouse) that is transgenic for human immunoglobulin genes; or antibodies prepared, expressed, created or isolated by any other means that involves splicing of human immunoglobulin gene sequences to other DNA sequences. Such recombinant human antibodies have variable and constant regions derived from human germline immunoglobulin sequences. In various embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. All such recombinant means are well known to those of ordinary skill in the art.

The term “anti-CCR8 antagonist antibody” (interchangeably termed “anti-CCR8 antibody”) refers to an antibody that is able to bind to CCR8 and inhibit CCR8 biological activity and/or downstream pathway(s) mediated by CCR8 signaling. An anti-CCR8 antagonist antibody encompasses antibodies that block, antagonize, suppress or reduce (including significantly) CCR8 biological activity, including downstream pathways mediated by CCR8 signaling, such as receptor binding and/or elicitation of a cellular response to CCR8. For purpose of the present invention, it will be explicitly understood that the term “anti-CCR8 antagonist antibody” encompasses all the previously identified terms, titles, and functional states and characteristics whereby the CCR8 itself, an CCR8 biological activity (including but not limited to its ability to mediate any aspect of headache), or the consequences of the biological activity, are substantially nullified, decreased, or neutralized in any meaningful degree. In some embodiment, an anti-CCR8 antagonist antibody binds CCR8 and prevents CCR8 binding to a CCR8 receptor. In other embodiments, an anti-CCR8 antibody binds CCR8 and prevents activation of a CCR8 receptor. Examples of anti-CCR8 antagonist antibodies are provided herein.

The term “epitope” as used herein includes any protein determinant capable of specific binding to an immunoglobulin or T-cell receptor or otherwise interacting with a molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between the protein and the interacting molecule (such as an antibody) occur linearly along the primary amino acid sequence of the protein. In a conformational epitope, the points of interaction occur across amino acid residues on the protein that are separated from one another. Once a desired epitope on an antigen is determined, it is possible to generate antibodies to that epitope, e.g., using the techniques described in the present disclosure. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen.

An antigen binding protein, including an antibody, “specifically binds” to an antigen if it binds to the antigen with a high binding affinity as determined by a dissociation constant (K_{D}, or corresponding Kb, as defined below) value of at least 1×10⁻⁶ M, or at least 1×10⁻⁷ M, or at least 1×10⁻⁸ M, or at least 1×10⁻⁹ M, or at least 1×10⁻¹⁰ M, or at least 1×10⁻¹¹ M. An antigen binding protein that specifically binds to the human antigen of interest may be able to bind to the same antigen of interest from other species as well, with the same or different affinities. The term “K_{D}” as used herein refers to the equilibrium dissociation constant of a particular antigen:antibody interaction.

The term “surface plasmon resonance” as used herein refers to an optical phenomenon that allows for the analysis of real-time biospecific interactions by detection of alterations in protein concentrations within a biosensor matrix, for example using the BIACORE™ system (Pharmacia Biosensor AB, Uppsala, Sweden and Piscataway, N.J.). For further descriptions, see Jonsson U. et al., Ann. Biol. Clin., 51:19-26, 1993; Jonsson U. et al., Biotechniques, 11:620-627, 1991; Jonsson B. et al., J. Mol. Recognit., 8:125-131, 1995; and Johnsson B. et al., Anal. Biochem, 198:268-277, 1991.

As used herein, the term “tumor microenvironment” refers to the cellular environment in which the tumor exists, including surrounding blood vessels, immune cells, fibroblasts, bone marrow-derived inflammatory cells, lymphocytes, signaling molecules and the extracellular matrix (ECM). Components in the tumor microenvironment can modulate the growth of tumor cells, e.g., their ability to progress and metastasize. The tumor microenvironment can also be influenced by the tumor releasing extracellular signals, promoting tumor angiogenesis and inducing peripheral immune tolerance.

The term “immunogenicity” as used herein refers to the ability of an antibody or antigen binding fragment to elicit an immune response (humoral or cellular) when administered to a recipient and includes, for example, the human anti-mouse antibody (HAMA) response. A HAMA response is initiated when T-cells from a subject make an immune response to the administered antibody. The T-cells then recruit B-cells to generate specific “anti-antibody” antibodies.

The term “immune cell” as used herein means any cell of hematopoietic lineage involved in regulating an immune response against an antigen (e.g., an autoantigen). In various embodiments, an immune cell is, e.g., a T cell, a B cell, a dendritic cell, a monocyte, a natural killer cell, a macrophage, Langerhan's cells, or Kuffer cells.

“Pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

As used herein, “treatment” (and grammatical variations thereof such as “treat” or “treating”) refers to clinical intervention in an attempt to alter the natural course of a disease in the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

The term “effective amount” or “therapeutically effective amount” as used herein refers to an amount of a compound or composition sufficient to treat a specified disorder, condition or disease such as ameliorate, palliate, lessen, and/or delay one or more of its symptoms. In reference to NHL and other cancers or other unwanted cell proliferation, an effective amount comprises an amount sufficient to: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective amount can be administered in one or more administrations.

“Adjuvant setting” refers to a clinical setting in which an individual has had a history of a proliferative disease, particularly cancer, and generally (but not necessarily) been responsive to therapy, which includes, but is not limited to, surgery (such as surgical resection), radiotherapy, and chemotherapy. However, because of their history of the proliferative disease (such as cancer), these individuals are considered at risk of development of the disease. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease when first treated.

The phrase “administering” or “cause to be administered” refers to the actions taken by a medical professional (e.g., a physician), or a person controlling medical care of a patient, that control and/or permit the administration of the agent(s)/compound(s) at issue to the patient. Causing to be administered can involve diagnosis and/or determination of an appropriate therapeutic regimen, and/or prescribing particular agent(s)/compounds for a patient. Such prescribing can include, for example, drafting a prescription form, annotating a medical record, and the like. Where administration is described herein, “causing to be administered” is also contemplated.

As used herein, the terms “co-administration”, “co-administered” and “in combination with”, referring to the fusion molecules of the invention and one or more other therapeutic agents, is intended to mean, and does refer to and include the following: simultaneous administration of such combination of fusion molecules of the invention and therapeutic agent(s) to an individual in need of treatment, when such components are formulated together into a single dosage form which releases said components at substantially the same time to said individual; substantially simultaneous administration of such combination of fusion molecules of the invention and therapeutic agent(s) to an individual in need of treatment, when such components are formulated apart from each other into separate dosage forms which are taken at substantially the same time by said individual, whereupon said components are released at substantially the same time to said individual; sequential administration of such combination of fusion molecules of the invention and therapeutic agent(s) to an individual in need of treatment, when such components are formulated apart from each other into separate dosage forms which are taken at consecutive times by said individual with a significant time interval between each administration, whereupon said components are released at substantially different times to said individual; and sequential administration of such combination of fusion molecules of the invention and therapeutic agent(s) to an individual in need of treatment, when such components are formulated together into a single dosage form which releases said components in a controlled manner whereupon they are concurrently, consecutively, and/or overlappingly released at the same and/or different times to said individual, where each part may be administered by either the same or a different route.

The terms “patient,” “individual,” and “subject” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the patient can be a human (e.g., adult male, adult female, adolescent male, adolescent female, male child, female child) under the care of a physician or other health worker in a hospital, psychiatric care facility, as an outpatient, or other clinical context. In various embodiments, the patient may be an immunocompromised patient or a patient with a weakened immune system including, but not limited to patients having primary immune deficiency, AIDS; cancer and transplant patients who are taking certain immunosuppressive drugs; and those with inherited diseases that affect the immune system (e.g., congenital agammaglobulinemia, congenital IgA deficiency). In various embodiments, the patient has an immunogenic cancer, including, but not limited to bladder cancer, lung cancer, melanoma, and other cancers reported to have a high rate of mutations (Lawrence et al., Nature, 499 (7457): 214-218, 2013).

The term “immunotherapy” refers to cancer treatments which include, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAG3, TIM-3, SIRP, CD40, CD47, Siglec 8, Siglec 9, Siglec 15, TIGIT and VISTA; treatment using bispecific T cell engaging antibodies (BiTE®) such as blinatumomab: treatment involving administration of biological response modifiers such as IL-2, IL-12, IL-15, IL-21, GM-CSF, IFN-α, IFN-β and IFN-γ; treatment using therapeutic vaccines such as sipuleucel-T; treatment using Bacilli Calmette-Guerin (BCG); treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

“Resistant or refractory cancer” refers to tumor cells or cancer that do not respond to previous anti-cancer therapy including, e.g., chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatment or respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, immunotherapy alone, surgery alone, or combinations thereof. For ease of description and not limitation, it will be understood that the refractory tumor cells are interchangeable with resistant tumor.

As used herein, “specific binding” is meant that the binding is selective for the antigen and can be discriminated from unwanted or non-specific interactions. The ability of an immunoglobulin to bind to a specific antigen can be measured either through an enzyme-linked immunosorbent assay (ELISA) or other techniques familiar to one of skill in the art, e.g., Surface Plasmon Resonance (SPR) technique.

The terms “affinity” or “binding affinity” as used herein refers to the strength of the sum total of non-covalent interactions between a single binding site of a molecule (e.g. an antibody) and its binding partner (e.g. an antigen). The affinity of a molecule X for its partner Y can generally be represented by the dissociation constant (K_{D}), which is the ratio of dissociation and association rate constants (koff and kon, respectively). A particular method for measuring affinity is Surface Plasmon Resonance (SPR).

The term “reduced binding”, as used herein refers to a decrease in affinity for the respective interaction, as measured for example by SPR. Conversely, “increased binding” refers to an increase in binding affinity for the respective interaction.

The term “polymer” as used herein generally includes, but is not limited to, homopolymers; copolymers, such as, for example, block, graft, random and alternating copolymers; and terpolymers; and blends and modifications thereof. Furthermore, unless otherwise specifically limited, the term “polymer” shall include all possible geometrical configurations of the material. These configurations include, but are not limited to isotactic, syndiotactic, and random symmetries.

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

A “vector” is a polynucleotide that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. A nucleotide sequence is “operably linked” to a regulatory sequence if the regulatory sequence affects the expression (e.g., the level, timing, or location of expression) of the nucleotide sequence.

A “host cell” is a cell that can be used to express a polynucleotide of the disclosure. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “isolated molecule” (where the molecule is, for example, a polypeptide or a polynucleotide) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “heterologous” as used herein refers to a composition or state that is not native or naturally found, for example, that may be achieved by replacing an existing natural composition or state with one that is derived from another source. Similarly, the expression of a protein in an organism other than the organism in which that protein is naturally expressed constitutes a heterologous expression system and a heterologous protein.

As used herein and in the appended claims, the singular forms “a,” “or,” and “the” include plural referents unless the context clearly dictates otherwise. It is understood that aspect and embodiments of the disclosure described herein include “consisting” and/or “consisting essentially of” aspects and embodiments.

Reference to “about” a value or parameter herein includes (and describes) variations that are directed to that value or parameter per se. For example, description referring to “about X” includes description of “X”.

### C—C Chemokine Receptor Type 8 (CCR8)

CCR8 is a G protein-coupled 7-transmembrane CC chemokine receptor protein expressed in the thymus, the spleen, etc. A gene encoding this protein resides on human chromosome 3p21. Human CCR8 consists of 355 amino acids (J. Immunol., 1996, Vol. 157, No. 7, p. 2759-63). CCL1 is known as an endogenous ligand for CCR8 (J. Biol. Chem., 1997, Vol. 272, No. 28, p. 17251-4). Human CCR8 cDNA is constituted by the nucleotide sequence represented by GenBank ACC No. NM_005201.3, and mouse CCR8 cDNA is constituted by the nucleotide sequence represented by GenBank ACC No. NM_007720.2.

The term “CCR8” as used herein includes human CCR8 (hCCR8), variants, isoforms, and species homologs of hCCR8, and analogs having at least one common epitope with hCCR8. In various embodiments, a hCCR8 as used herein may comprise the amino acid sequence set forth in SEQ ID NO: 1:

(SEQ ID NO: 1)
MDYTLDLSVTTVTDYYYPDIFSSPCDAELIQTNGKLLLAVFYCLLF
VFSLLGNSLVILVLVVCKKLRSITDVYLLNLALSDLLFVFSFPFQ
TYYLLDQWVFGTVMCKVVSGFYYIGFYSSMFFITLMSVDRYLAVV
HAVYALKVRTIRMGTTLCLAVWLTAIMATIPLLVFYQVASEDGVL
QCYSFYNQQTLKWKIFTNFKMNILGLLIPFTIFMFCYIKILHQLK
RCQNHNKTKAIRLVLIVVIASLLFWVPFNVVLFLTSLHSMHILDG
CSISQQLTYATHVTEIISFTHCCVNPVIYAFVGEKFKKHLSEIFQ
KSCSQIFNYLGRQMPRESCEKSSSCQQHSSRSSSVDYIL

In various embodiments, a CCR8 comprises an amino acid sequence that shares an observed homology of, e.g., at least 70%, at least 75%, at least 80%, at least 85%, at least 90%, at least 95%, at least 96%, at least 97%, at least 98%, or at least 99% with the human CCR8 sequence of SEQ ID NO: 1. In some embodiments, the has at least 70%, at least 75%, at least 80%, at least 85%, at least 90%, at least 95%, at least 96%, at least 97%, at least 98%, at least 99%, at least 1×, at least 1.5×, at least 2×, at least 2.5×, or at least 3× activity of the human CCR8 of SEQ ID NO: 1. Variants of CCR8 may be described herein by reference to the addition, deletion, or substitution of amino acid residue present at a given position in the 360 amino acid sequence of SEQ ID NO: 1. Thus, for example, the term “T10S” indicates that the “T” (threonine, in standard single letter code) residue at position 10 in SEQ ID NO: 1 has been substituted with a “S” (serine, in standard single letter code).

### Antibodies

Methods of generating novel antibodies that bind to human CCR8 are known to those skilled in the art. For example, a method for generating a monoclonal antibody that binds specifically to an CCR8 may comprise administering to a mouse an amount of an immunogenic composition comprising the CCR8 effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monoclonal antibody that binds specifically to the CCR8. Once obtained, a hybridoma can be propagated in a cell culture, optionally in culture conditions where the hybridoma-derived cells produce the monoclonal antibody that binds specifically to CCR8. The monoclonal antibody may be purified from the cell culture. A variety of different techniques are then available for testing antigen:antibody interactions to identify particularly desirable antibodies.

Other suitable methods of producing or isolating antibodies of the requisite specificity can used, including, for example, methods which select recombinant antibody from a library, or which rely upon immunization of transgenic animals (e.g., mice) capable of producing a full repertoire of human antibodies. See e.g., Jakobovits et al., Proc. Natl. Acad. Sci. USA, 90:2551-2555, 1993; Jakobovits et al., Nature, 362:255-258, 1993; Lonberg et al., U.S. Pat. No. 5,545,806; Surani et al., U.S. Pat. No. 5,545,807.

Antibodies can be engineered in numerous ways. They can be made as single-chain antibodies (including small modular immunopharmaceuticals or SMIPs™), Fab and F(ab′)₂ fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., Science, 240:1041-1043, 1988; Liu et al., PNAS USA, 84:3439-3443, 1987; Liu et al., J. Immunol. 139:3521-3526, 1987; Sun et al., PNAS USA, 84:214-218, 1987; Nishimura et al., Canc. Res. 47:999-1005, 1987; Wood et al., Nature 314:446-449, 1985; and Shaw et al., J. Natl Cancer Inst., 80:1553-1559, 1988).

Methods for humanizing antibodies have been described in the art. In practice, humanized antibodies are typically human antibodies in which some hypervariable region residues and possibly some framework region residues are substituted by residues from analogous sites in rodent antibodies. Accordingly, such “humanized” antibodies are chimeric antibodies wherein substantially less than an intact human variable region has been substituted by the corresponding sequence from a nonhuman species. To a degree, this can be accomplished in connection with techniques of humanization and display techniques using appropriate libraries. It will be appreciated that murine antibodies or antibodies from other species can be humanized or primatized using techniques well known in the art (see e.g., Winter et al., Immunol Today, 14:43-46, 1993; and Wright et al., Crit. Reviews in Immunol., 12125-168, 1992). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (Liu et al., P.N.A.S. 84:3439, 1987; J. Immunol. 139:3521, 1987). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions to genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. In various embodiments, the isotype is selected from the group consisting of IgG1, IgG2, IgG3 and IgG4. Either of the human light chain constant regions, kappa or lambda, may be used. The chimeric, humanized antibody is then expressed by conventional methods.

U.S. Pat. No. 5,693,761 to Queen et al, discloses a refinement on Winter et al. for humanizing antibodies, and is based on the premise that ascribes avidity loss to problems in the structural motifs in the humanized framework which, because of steric or other chemical incompatibility, interfere with the folding of the CDRs into the binding-capable conformation found in the mouse antibody. To address this problem, Queen teaches using human framework sequences closely homologous in linear peptide sequence to framework sequences of the mouse antibody to be humanized. Accordingly, the methods of Queen focus on comparing framework sequences between species. Typically, all available human variable region sequences are compared to a particular mouse sequence and the percentage identity between correspondent framework residues is calculated. The human variable region with the highest percentage is selected to provide the framework sequences for the humanizing project. Queen also teaches that it is important to retain in the humanized framework, certain amino acid residues from the mouse framework critical for supporting the CDRs in a binding-capable conformation. Potential criticality is assessed from molecular models. Candidate residues for retention are typically those adjacent in linear sequence to a CDR or physically within 6 Å of any CDR residue.

In other approaches, the importance of particular framework amino acid residues is determined experimentally once a low-avidity humanized construct is obtained, by reversion of single residues to the mouse sequence and assaying antigen-binding as described by Riechmann et al, 1988. Another example approach for identifying important amino acids in framework sequences is disclosed by U.S. Pat. No. 5,821,337 to Carter et al, and by U.S. Pat. No. 5,859,205 to Adair et al. These references disclose specific Kabat residue positions in the framework, which, in a humanized antibody may require substitution with the correspondent mouse amino acid to preserve avidity.

Another method of humanizing antibodies, referred to as “framework shuffling”, relies on generating a combinatorial library with nonhuman CDR variable regions fused in frame into a pool of individual human germline frameworks (Dall'Acqua et al., Methods, 36:43, 2005). The libraries are then screened to identify clones that encode humanized antibodies which retain good binding.

The choice of human variable regions, both light and heavy, to be used in making the desired humanized antibodies is very important to reduce antigenicity. According to the so-called “best-fit” method, the sequence of the variable region of a rodent antibody is screened against the entire library of known human variable-domain sequences. The human sequence that is closest to that of the rodent is then accepted as the human framework region (framework region) for the humanized antibody (Sims et al., J. Immunol., 151:2296, 1993; Chothia et al., J. Mol. Biol., 196:901, 1987). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA, 89:4285, 1992; Presta et al., J. Immunol., 151:2623, 1993).

The choice of nonhuman residues to substitute into the human variable region can be influenced by a variety of factors. These factors include, for example, the rarity of the amino acid in a particular position, the probability of interaction with either the CDRs or the antigen, and the probability of participating in the interface between the light and heavy chain variable domain interface. (See, for example, U.S. Pat. Nos. 5,693,761, 6,632,927, and 6,639,055). One method to analyze these factors is through the use of three-dimensional models of the nonhuman and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind its antigen. In this way, nonhuman residues can be selected and substituted for human variable region residues in order to achieve the desired antibody characteristic, such as increased affinity for the target antigen(s).

Methods for making fully human antibodies have been described in the art. By way of example, a method for producing an anti-CCR8 antibody or antigen-binding fragment thereof comprises the steps of synthesizing a library of human antibodies on phage, screening the library with CCR8 or an antibody-binding portion thereof, isolating phage that bind CCR8, and obtaining the antibody from the phage. By way of another example, one method for preparing the library of antibodies for use in phage display techniques comprises the steps of immunizing a non-human animal comprising human immunoglobulin loci with CCR8 or an antigenic portion thereof to create an immune response, extracting antibody-producing cells from the immunized animal; isolating RNA encoding heavy and light chains of antibodies of the invention from the extracted cells, reverse transcribing the RNA to produce cDNA, amplifying the cDNA using primers, and inserting the cDNA into a phage display vector such that antibodies are expressed on the phage. Recombinant anti-CCR8 antibodies of the invention may be obtained in this way.

Recombinant human anti-CCR8 antibodies of the invention can also be isolated by screening a recombinant combinatorial antibody library. Preferably the library is a scFv phage display library, generated using human VL and VH cDNAs prepared from mRNA isolated from B cells. Methods for preparing and screening such libraries are known in the art. Kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology 9:1370-1372 (1991); Hay et al., Hum. Antibod. Hybridomas 3:81-85, 1992; Huse et al., Science 246:1275-1281, 1989; McCafferty et al., Nature 348:552-554, 1990; Griffiths et al., EMBO J. 12:725-734, 1993; Hawkins et al., J. Mol. Biol. 226:889-896, 1992; Clackson et al., Nature 352:624-628, 1991; Gram et al., Proc. Natl. Acad. Sci. USA 89:3576-3580, 1992; Garrad et al., Bio/Technology 9:1373-1377, 1991; Hoogenboom et al., Nuc. Acid Res. 19:4133-4137, 1991; and Barbas et al., Proc. Natl. Acad. Sci. USA 88:7978-7982, 1991, each incorporated herein by reference for purposes of teaching preparation and screening of phase display libraries.

Human antibodies are also produced by immunizing a non-human, transgenic animal comprising within its genome some or all of human immunoglobulin heavy chain and light chain loci with a human IgE antigen, e.g., a XenoMouse™ animal (Abgenix, Inc./Amgen, Inc.—Fremont, Calif.). XenoMouse™ mice are engineered mouse strains that comprise large fragments of human immunoglobulin heavy chain and light chain loci and are deficient in mouse antibody production. See, e.g., Green et al., Nature Genetics 7:13-21, 1994; and U.S. Pat. Nos. 5,916,771, 5,939,598, 5,985,615, 5,998,209, 6,075, 181, 6,091,001, 6,114,598, 6,130,364, 6,162,963 and 6,150,584. See also WO 91/10741, WO 94/02602, WO 96/34096, WO 96/33735, WO 98/16654, WO 98/24893, WO 98/50433, WO 99/45031, WO 99/53049, WO 00/09560, and WO 00/037504. XenoMouse™ mice produce an adult-like human repertoire of fully human antibodies and generate antigen-specific human antibodies. In some embodiments, the XenoMouse™ mice contain approximately 80% of the human antibody V gene repertoire through introduction of megabase sized, germline configuration fragments of the human heavy chain loci and kappa light chain loci in yeast artificial chromosome (YAC). In other embodiments, XenoMouse™ mice further contain approximately all of the human lambda light chain locus. See Mendez et al., Nature Genetics 15:146-156, 1997, Green and Jakobovits, J. Exp. Med. 188:483-495 (1998), and WO 98/24893 (each incorporated by reference in its entirety for purposes of teaching the preparation of fully human antibodies). In another aspect, the present invention provides a method for making anti-CCR8 antibodies from non-human, non-mouse animals by immunizing non-human transgenic animals that comprise human immunoglobulin loci with a CCR8 antigen. One can produce such animals using the methods described in the above-cited documents.

### Characterization of Antibody Binding to Antigen

Antibodies of the present invention can be tested for binding to human CCR8 by, for example, standard ELISA. As an example, microtiter plates are coated with purified CCR8 in PBS or cells overexpressing human CCR8, and then blocked with 5% bovine serum albumin in PBS. Dilutions of antibody (e.g., dilutions of plasma from CCR8-immunized mice) are added to each well and incubated for 1-2 hours at 37° C. or 4° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml) and analyzed at OD of 405-650 nm. Preferably, mice which develop the highest titers will be used for fusions. An ELISA can also be used to screen for hybridomas that show positive reactivity with CCR8 immunogen. Hybridomas that bind with high avidity to CCR8 are subcloned and further characterized. One clone from each hybridoma, which retains the reactivity of the parent cells (by ELISA), can be chosen for making a 5-10 vial cell bank stored at −140° C., and for antibody purification.

To determine if the selected anti-CCR8 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using CCR8 coated-ELISA plates as described above. Biotinylated mAb binding can be detected with a strep-avidin-alkaline phosphatase probe. To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-CCR8 human IgGs can be further tested for reactivity with CCR8 antigen by Western blotting. Briefly, CCR8 can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

### Identification of Anti-CCR8 Antibodies

The present invention provides monoclonal antibodies, and antigen-binding fragments thereof, that specifically bind to CCR8 antigen.

Further included in the present invention are antibodies that bind to the same epitope as the anti-CCR8 antibodies of the present invention. To determine if an antibody can compete for binding to the same epitope as the epitope bound by the anti-CCR8 antibodies of the present invention, a cross-blocking assay, e.g., a competitive ELISA, can be performed. In an exemplary competitive ELISA, CCR8 coated on the wells of a microtiter plate is pre-incubated with or without candidate competing antibody and then the biotin labeled anti-CCR8 antibody of the invention is added. The amount of labeled anti-CCR8 antibody bound to the CCR8 antigen in the wells is measured using avidin-peroxidase conjugate and appropriate substrate. The antibody can be labeled with a radioactive or fluorescent label or some other detectable and measurable label. The amount of labeled anti-CCR8 antibody that bound to the antigen will have an indirect correlation to the ability of the candidate competing antibody (test antibody) to compete for binding to the same epitope, i.e., the greater the affinity of the test antibody for the same epitope, the less labeled antibody will be bound to the antigen-coated wells. A candidate competing antibody is considered an antibody that binds substantially to the same epitope or that competes for binding to the same epitope as an anti-CCR8 antibody of the invention if the candidate antibody can block binding of the CCR8 antibody by at least 20%, preferably by at least 20-50%, even more preferably, by at least 50% as compared to the control performed in parallel in the absence of the candidate competing antibody. It will be understood that variations of this assay can be performed to arrive at the same quantitative value.

The amino acid sequences of the heavy chain variable region CDRs and the light chain variable region CDRs of various murine mAbs, MAbs A1-A19, generated as described herein, is shown below in Table 2.

TABLE 2
Heavy Chain CDRs

| Ab | HCDR1 | HCDR2 | HCDR3 |
|---|---|---|---|
| A1 | AYAMN | RIRSKSNNYATYYADSVKD | GGTYGSSSYFDY |
| | (SEQ ID NO: 3) | (SEQ ID NO: 4) | (SEQ ID NO: 5) |
| A2 | AYAMN | RIRSKSNNYATYYADSVKD | GGTYGSTSYFDY |
| | (SEQ ID NO: 3) | (SEQ ID NO: 4) | (SEQ ID NO: 6) |
| A3 | TYAMN | RIRSKSNNYATYYADSVKA | GGTYGSTSYFDY |
| | (SEQ ID NO: 7) | (SEQ ID NO: 8) | (SEQ ID NO: 6) |
| A4 | DYNMD | AINPNNGGTGYTQKFKG | RGVYMFAY |
| | (SEQ ID NO: 9) | (SEQ ID NO: 10) | (SEQ ID NO: 11) |
| A5 | TYAMN | RIRTKSNNYATYYADSVKD | GGSGIKYVRYFDV |
| | (SEQ ID NO: 7) | (SEQ ID NO: 35) | (SEQ ID NO: 39) |
| A6 | AYAMN | RIRTKSNNYATYYADSVKD | GGSGIRYVKYFDV |
| | (SEQ ID NO: 3) | (SEQ ID NO: 35) | (SEQ ID NO: 40) |
| A7 | AYAMN | RIRTKSNNYATYYADSVKD | GGSGIRYVKYFDV |
| | (SEQ ID NO: 3) | (SEQ ID NO: 35) | (SEQ ID NO: 40) |
| A8 | TYAMN | RIRSKSNNYATYYADSVKD | GGSGISYVRYFDV |
| | (SEQ ID NO: 7) | (SEQ ID NO: 4) | (SEQ ID NO: 41) |
| A9 | TYAMN | RIRTKSNNYATYYADSVKD | GGSGLNYVRYFDV |
| | (SEQ ID NO: 7) | (SEQ ID NO: 35) | (SEQ ID NO: 42) |
| A10 | TYAMN | RIRTKSNNYATYYADSVKD | GGSGLRYVRYFDV |
| | (SEQ ID NO: 7) | (SEQ ID NO: 35) | (SEQ ID NO: 43) |
| A11 | TYAMN | RIRTKSNNYATYYADSVKD | GGSGLRYVRYFDV |
| | (SEQ ID NO: 7) | (SEQ ID NO: 35) | (SEQ ID NO: 43) |
| A12 | TYAMN | RIRSKSNNYATYYADSVKD | QTYGSRDYAMDY |
| | (SEQ ID NO: 7) | (SEQ ID NO: 4) | (SEQ ID NO: 44) |
| A13 | AYAMN | RIRSKSNNYATYYADSVKD | QTYGSRDYAMDY |
| | (SEQ ID NO: 3) | (SEQ ID NO: 4) | (SEQ ID NO: 44) |
| A14 | TYAMN | RIRSKSNNYATYYADSVKD | GGSGIRYVRYFDV |
| | (SEQ ID NO: 7) | (SEQ ID NO: 4) | (SEQ ID NO: 45) |
| A15 | AYAMN | RIRSKSNNFATYYADSVKD | QTYGSRDYAMDY |
| | (SEQ ID NO: 3) | (SEQ ID NO: 38) | (SEQ ID NO: 44) |
| A16 | TYAMN | RIRTKSNNYATYYADSVKD | GGSGLNYVRYFDV |
| | (SEQ ID NO: 7) | (SEQ ID NO: 35) | (SEQ ID NO: 42) |
| A17 | TYAMN | RIRTKSNNYATFYADSVKD | GGSGIRYVRYFDV |
| | (SEQ ID NO: 7) | (SEQ ID NO: 36) | (SEQ ID NO: 45) |
| A18 | TYAMN | RIRTKSNNYATYYAASVKD | GGSGLNYVRYFDV |
| | (SEQ ID NO: 7) | (SEQ ID NO: 37) | (SEQ ID NO: 42) |
| A19 | AYAMN | RIRSKSNNFATYYADSVKD | QTYGSRDYAMDY |
| | (SEQ ID NO: 3) | (SEQ ID NO: 38) | (SEQ ID NO: 44) |

Light Chain CDRs

| Ab | LCDR1 | LCDR2 | LCDR3 |
|---|---|---|---|
| A1 | RSSKSLLHSNGNTYLY | RMSNLAS | MQHLEYPFT |
| | (SEQ ID NO: 12) | (SEQ ID NO: 13) | (SEQ ID NO: 14) |
| A2 | RSSKSLLHSNGNTYLY | RMSNLAS | MQHLEYPFT |
| | (SEQ ID NO: 12) | (SEQ ID NO: 13) | (SEQ ID NO: 14) |
| A3 | RSSKSLLHSNGNTYLY | RMSNLAS | MQHLEYPFT |
| | (SEQ ID NO: 12) | (SEQ ID NO: 13) | (SEQ ID NO: 14) |
| A4 | KSSQSLLHSDGKTYLN | LVSKLDS | WQGTHFPYT |
| | (SEQ ID NO: 15) | (SEQ ID NO: 16) | (SEQ ID NO: 17) |
| A5 | RSSKSLLHSNGNTYLY | RMSNLAS | MQHLEYPFT |
| | (SEQ ID NO: 12) | (SEQ ID NO: 13) | (SEQ ID NO: 14) |
| A6 | RSSKSLLHSNGNTYLY | RMSNLAS | MQHLEYPFT |
| | (SEQ ID NO: 12) | (SEQ ID NO: 13) | (SEQ ID NO: 14) |
| A7 | RSSKSLLHSNGNTYLY | RMSNLAS | MQHLEYPFT |
| | (SEQ ID NO: 12) | (SEQ ID NO: 13) | (SEQ ID NO: 14) |
| A8 | RSSKSLLHSNGNTYLY | RMSNLAS | MQHLEYPFT |
| | (SEQ ID NO: 12) | (SEQ ID NO: 13) | (SEQ ID NO: 14) |
| A9 | RSSKSLLHSNGNTYLY | RMSNLAS | MQHLEYPFT |
| | (SEQ ID NO: 12) | (SEQ ID NO: 13) | (SEQ ID NO: 14) |
| A10 | RSSKSLLHSNGNTYLY | RMSNLAS | MQHLEYPFT |
| | (SEQ ID NO: 12) | (SEQ ID NO: 13) | (SEQ ID NO: 14) |
| A11 | RSSKSLLHSNGNTYLY | RMSNLAS | MQHLEYPFT |
| | (SEQ ID NO: 12) | (SEQ ID NO: 13) | (SEQ ID NO: 14) |
| A12 | RSSQSLVHSNGNTYLH | KVSNRFS | CQSTHVPPYT |
| | (SEQ ID NO: 46) | (SEQ ID NO: 48) | (SEQ ID NO: 50) |
| A13 | RSSQSLVHSNGNTYLH | KVSNRFS | CQSTHVPPYT |
| | (SEQ ID NO: 46) | (SEQ ID NO: 48) | (SEQ ID NO: 50) |
| A14 | RSSKSLQHSNGNIYLY | RMSNLAS | MQHLEYPFT |
| | (SEQ ID NO: 47) | (SEQ ID NO: 13) | (SEQ ID NO: 14) |
| A15 | RSSQSLVHSNGNTYLH | KVSNRFS | SQSTHVPPYT |
| | (SEQ ID NO: 46) | (SEQ ID NO: 48) | (SEQ ID NO: 51) |
| A16 | RSSKSLLHSNGNTYLY | RMSNLAS | MQHLEYPFT |
| | (SEQ ID NO: 12) | (SEQ ID NO: 13) | (SEQ ID NO: 14) |
| A17 | RSSKSLLHSNGNTYLY | RMSDLAS | MQHLEYPFT |
| | (SEQ ID NO: 12) | (SEQ ID NO: 49) | (SEQ ID NO: 14) |
| A18 | RSSKSLLHSNGNTYLY | RMSNLAS | MQHLEYPFT |
| | (SEQ ID NO: 12) | (SEQ ID NO: 13) | (SEQ ID NO: 14) |
| A19 | RSSQSLVHSNGNTYLH | KVSNRFS | SQNTHVPPYT |
| | (SEQ ID NO: 46) | (SEQ ID NO: 48) | (SEQ ID NO: 52) |

In various embodiments of the present invention, the antibody or antigen-binding fragment is a murine antibody comprising the heavy chain variable region sequence and light chain variable region sequence combination as set forth in Table 3:

TABLE 3
Mouse Ab clones

| Ab | Clone | HCVR | LCVR |
|---|---|---|---|
| MAb 1 | 41E1C2A5 | SEQ ID NO: 18 | SEQ ID NO: 19 |
| MAb 2 | 46A5C4B1 | SEQ ID NO: 20 | SEQ ID NO: 21 |
| MAb 3 | 80E4D1F11 | SEQ ID NO: 22 | SEQ ID NO: 23 |
| MAb 4 | 419C7B3B2 | SEQ ID NO: 24 | SEQ ID NO: 25 |
| MAb 5 | 508C11A12 | SEQ ID NO: 53 | SEQ ID NO: 68 |
| MAb 6 | 529D10C10 | SEQ ID NO: 54 | SEQ ID NO: 68 |
| MAb 7 | 536H9D10 | SEQ ID NO: 55 | SEQ ID NO: 68 |
| MAb 8 | 506G6G5 | SEQ ID NO: 56 | SEQ ID NO: 69 |
| MAb 9 | 531A6A3 | SEQ ID NO: 57 | SEQ ID NO: 69 |
| MAb 10 | 516D7D12 | SEQ ID NO: 58 | SEQ ID NO: 70 |
| MAb 11 | 525F2F3F11 | SEQ ID NO: 59 | SEQ ID NO: 70 |
| MAb 12 | 530H3G7D9 | SEQ ID NO: 60 | SEQ ID NO: 71 |
| MAb 13 | 531B9B1C9 | SEQ ID NO: 61 | SEQ ID NO: 71 |
| MAb 14 | 507B6B10 | SEQ ID NO: 62 | SEQ ID NO: 72 |
| MAb 15 | 509H4E10B3 | SEQ ID NO: 63 | SEQ ID NO: 73 |
| MAb 16 | 526G1C12 | SEQ ID NO: 64 | SEQ ID NO: 74 |
| MAb 17 | 531A3E12H8 | SEQ ID NO: 65 | SEQ ID NO: 75 |
| MAb 18 | 504E12D8D12 | SEQ ID NO: 66 | SEQ ID NO: 76 |
| MAb 19 | 538H5G10 | SEQ ID NO: 67 | SEQ ID NO: 77 |

In various embodiments, antibodies of the present invention include antibodies that bind to the same epitope as murine antibodies MAb1-MAb19.

In various embodiments of the present invention, the antibody or antigen-binding fragment is a murine-human chimeric antibody (derived from murine antibody A1 (“41E1C2A5”) and human IgG1) comprising the heavy chain sequence of SEQ ID NO: 26:

(SEQ ID NO: 26)
EVQLVESGGGLVQPKGSLKLSCAASGFSFNAYAMNWVRQAPGKGLE
WVARIRSKSNNYATYYADSVKDRFTISRDDSETMLYLQMNNLKTE
DTAMYFCVRGGTYGSSSYFDYWGQGTTLTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKKVEPKSC
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GK

and the light chain sequence of SEQ ID NO: 27:

(SEQ ID NO: 27)
DIVMTQAAPSVPVTPGESVSIPCRSSKSLLHSNGNTYLYWFLQRP
GQSPQLLIYRMSNLASGVPDRFSGSGSGTAFTLRISRVEAEDVGV
YYCMQHLEYPFTFGGGTKLQIRRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC

In various embodiments of the present invention, the antibody or antigen-binding fragment is a murine-human chimeric antibody (derived from murine antibody A3 (“80E4D1F11”) and human IgG1) comprising the heavy chain sequence of SEQ ID NO: 28:

(SEQ ID NO: 28)
EVQLVESGGGLVQPKGSLKLSCAASGFSFNTYAMNWVRQAPGKGLE
WVARIRSKSNNYATYYADSVKARFTISRDDSESMLYLQMNNLKTE
DTAMYFCVRGGTYGSTSYFDYWGQGTTLTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKKVEPKSC
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GK

and the light chain sequence of SEQ ID NO: 29:

(SEQ ID NO: 29)
DIVMTQAAPSVPVTPGESVSISCRSSKSLLHSNGNTYLYWFLQRP
GQSPQLLIYRMSNLASGVPDRFSGSGSGTAFTLRISRVEAEDVGV
YYCMQHLEYPFTFGGGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC

In various embodiments of the present invention, the antibody or antigen-binding fragment is a murine-human chimeric antibody (derived from murine antibody A4 (“419C7B3B2”) and human IgG1) comprising the heavy chain sequence of SEQ ID NO: 30:

(SEQ ID NO: 30)
EVQLQQSGPELVKPGSSVKISCKASGYTFTDYNMDWVKQSHGKSL
EWIGAINPNNGGTGYTQKFKGKATLTVDKSSSTAFMELRSLTSED
SAVYYCARRGVYMFAYWGQGTLVTVSAASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPGK

and the light chain sequence of SEQ ID NO: 31:

(SEQ ID NO: 31)
DVVMTQTPLTLSVTIGQPASISCKSSQSLLHSDGKTYLNWLLQRP
GQSPKRLIYLVSKLDSGVPDRFTGSGSGTDFTLKISRVEAEDLGV
YYCWQGTHFPYTFGGGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC

In various embodiments of the present invention, the antibody or antigen-binding fragment is a murine-human chimeric antibody (derived from murine antibody A18 (“504E12D8D12”) and human IgG1) comprising the heavy chain sequence of SEQ ID NO: 78:

(SEQ ID NO: 78)
EVQLVESGGGLVQPKGSLKLSCAASGFSFNTYAMNWVRQAPGKGLE
WVARIRTKSNNYATYYAASVKDRFTISRDDSETMLYLQMNNLKTE
DTAMYYCVRGGSGLNYVRYFDVWGTGTTVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
RDELTKNQVSLTCLVKGFYPSDIAVEWESNGQPENNYKTTPPVLD
SDGSFFLYSKLTVDKSRWQQGNVFSCSVMHEALHNHYTQKSLSLS
PGK

and the light chain sequence of SEQ ID NO: 79:

(SEQ ID NO: 79)
DIVMTQAAPSVFVIPGESVSISCRSSKSLLHSNGNTYLYWFLQRP
GQSPQLLIYRMSNLASGVPDRFSGSGSGSAFTLRISRVEAEDVGV
YYCMQHLEYPFTFGSGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC

In various embodiments of the present invention, the antibody or antigen-binding fragment is a murine-human chimeric antibody (derived from murine antibody A10 (“516D7D12”) and human IgG1) comprising the heavy chain sequence of SEQ ID NO: 80:

(SEQ ID NO: 80)
EVQLVESGGGLVQPKGSLKLSCAASGFSFNTYAMNWVRQAPGKGLEWVA
RIRTKSNNYATYYADSVKDRFTISRDDSESMLYLQMNNLKTEDTAMYYC
VRGGSGLRYVRYFDVWGTGTTVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHN
HYTQKSLSLSPGK

and the light chain sequence of SEQ ID NO: 81:

(SEQ ID NO: 81)
DIVMTQATPSVPVTPGESVSISCRSSKSLLHSNGNTYLYWFLQRPGQSP
QLLIYRMSNLASGVPERFSGSGSGSAFTLRVSRVEAEDVGVYYCMQHLE
YPFTFGSGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

In various embodiments of the present invention, the antibody or antigen-binding fragment is a murine-human chimeric antibody (derived from murine antibody A11 (“525F2F3F11”) and human IgG1) comprising the heavy chain sequence of SEQ ID NO: 82:

(SEQ ID NO: 82)
EVQLVESGGGLVQPKGSLKLSCAASGFSFNTYAMNWVRQAPGKGLEWVA
RIRTKSNNYATYYADSVKDRFTISRDDSENMLYLQMNNLKTEDTAMYYC
VRGGSGLRYVRYFDVWGTGTTVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHN
HYTQKSLSLSPGK

and the light chain sequence of SEQ ID NO: 83:

(SEQ ID NO: 83)
DIVMTQATPSVPVTPGESVSISCRSSKSLLHSNGNTYLYWFLQRPGQSP
QLLIYRMSNLASGVPERFSGSGSGSAFTLRVSRVEAEDVGVYYCMQHLE
YPFTFGSGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

In various embodiments of the present invention, the antibody or antigen-binding fragment is a murine-human chimeric antibody (derived from murine antibody A13 (“531B9B1C9”) and human IgG1) comprising the heavy chain sequence of SEQ ID NO: 84:

(SEQ ID NO: 84)
EVQLVESGGGLVQPKGSLKLSCAASGFSFNAYAMNWVRQAPGKGLDWVA
RIRSKSNNYATYYADSVKDRFTISRDDSESMLYLQMNNLKTEDTAMYFC
VRQTYGSRDYAMDYWGQGTSVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK

and the light chain sequence of SEQ ID NO: 85:

(SEQ ID NO: 85)
DVVMTQTPLSLPVSLGDQASISCRSSQSLVHSNGNTYLHWYLQKPGQSP
KLLIYKVSNRFSGVPDRFSGSGSGTDFTLKISRVEAEDLGVYFCCQSTH
VPPYTFGGGTKLEIKRTVAAPSVFIFPPSDEQLKSGTASVVCLLNNFYP
REAKVQWKVDNALQSGNSQESVTEQDSKDSTYSLSSTLTLSKADYEKHK
VYACEVTHQGLSSPVTKSFNRGEC

Antibodies or antigen-binding fragments thereof of the invention can comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a IgA-, IgD-, IgE-, IgG- and IgM-type heavy chain constant region. In various embodiments, the light or heavy chain constant region is a fragment, derivative, variant, or mutein of a naturally occurring constant region.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lanitto et al., Methods Mol. Biol. 178:303-16, 2002.

In various embodiments, an antibody of the invention further comprises a light chain kappa or lambda constant domain, or a fragment thereof, and further comprises a heavy chain constant domain, or a fragment thereof. Sequences of the light chain constant region and heavy chain constant region used in the exemplified antibodies, and polynucleotides encoding them, are provided below.

Light Chain (Kappa) Constant Region
(SEQ ID NO: 32)
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSFNRGEC
Light Chain (Lambda) Constant Region
(SEQ ID NO: 33)
GQPKAAPSVTLFPPSSEELQANKATLVCLISDFYPGAVTVAWKADSSP
VKAGVETTTPSKQSNNKYAASSYLSLTPEQWKSHRSYSCQVTHEGSTV
EKTVAPTECS
Heavy Chain Constant Region
(SEQ ID NO: 34)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPGK

In various embodiments of the present invention, the antibody or antigen-binding fragment is a humanized antibody (“41E1C2A5-HC3-LC4”) comprising the heavy chain sequence of SEQ ID NO: 86:

(SEQ ID NO: 86)
EVQLVESGGGLVQPGGSLKLSCAASGFSFNAYAMNWVRQASGKGLEWVA
RIRSKSNNYATYYADSVKDRFTISRDDSKNTAYLQMNSLKTEDTAVYFC
VRGGTYGSSSYFDYWGQGTTVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK

and the light chain sequence of SEQ ID NO: 87:

(SEQ ID NO: 87)
DIVMTQSPLSLPVTPGEPASIPCRSSKSLLHSNGNTYLYWFLQKPGQSP
QLLIYRMSNLASGVPDRFSGSGSGTAFTLKISRVEAEDVGVYYCMQHLE
YPFTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

In various embodiments of the present invention, the antibody or antigen-binding fragment is a humanized antibody (“41E1C2A5-HC4-LC2”) comprising the heavy chain sequence of SEQ ID NO: 88:

(SEQ ID NO: 88)
EVQLVESGGGLVQPGGSLKLSCAASGFSFNAYAMNWVRQASGKGLEWVA
RIRSKSNNYATYYADSVKDRFTISRDDSENTAYLQMNSLKTEDTAVYFC
VRGGTYGSSSYFDYWGQGTTVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK

and the light chain sequence of SEQ ID NO: 89:

(SEQ ID NO: 89)
DIVMTQSPLSLPVTPGEPASISCRSSKSLLHSNGNTYLYWFLQKPGQSP
QLLIYRMSNLASGVPDRFSGSGSGTAFTLKISRVEAEDVGVYYCMQHLE
YPFTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

In various embodiments of the present invention, the antibody or antigen-binding fragment is a humanized antibody (“504E12D8D12-HC1-LC1”) comprising the heavy chain sequence of SEQ ID NO: 90:

(SEQ ID NO: 90)
EVQLVESGGGLVQPGGSLKLSCAASGFSFNTYAMNWVRQASGKGLEWVG
RIRTKSNNYATYYAASVKDRFTISRDDSKNTAYLQMNSLKTEDTAVYYC
TRGGSGLNYVRYFDVWGQGTTVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHN
HYTQKSLSLSPGK

and the light chain sequence of SEQ ID NO: 91:

(SEQ ID NO: 91)
DIVMTQTPPSLPVNPGEPASISCRSSKSLLHSNGNTYLYWYLQKPGQSP
QLLIYRMSNLASGVPDRFSGSGSGSDFTLKISWVEAEDVGVYYCMQHLE
YPFTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

In various embodiments of the present invention, the antibody or antigen-binding fragment is a humanized antibody (“504E12D8D12-HC1-LC1 (G34A)”) comprising the heavy chain sequence of SEQ ID NO: 90 and the light chain sequence of SEQ ID NO: 92:

(SEQ ID NO: 92)
DIVMTQTPPSLPVNPGEPASISCRSSKSLLHSNANTYLYWYLQKPGQSP
QLLIYRMSNLASGVPDRFSGSGSGSDFTLKISWVEAEDVGVYYCMQHLE
YPFTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

Antibodies of the present invention may also be described or specified in terms of their cross-reactivity. Antibodies that bind CCR8s, which have at least 95%, at least 90%, at least 85%, at least 80%, at least 75%, at least 70%, at least 65%, at least 60%, at least 55%, and at least 50% identity (as calculated using methods known in the art and described herein) to human CCR8 are also included in the present invention.

Further included in the present invention are antibodies that bind to the same epitope as the anti-CCR8 antibodies of the present invention. To determine if an antibody can compete for binding to the same epitope as the epitope bound by the anti-CCR8 antibodies of the present invention, a cross-blocking assay, e.g., a competitive ELISA, can be performed. In an exemplary competitive ELISA, CCR8 coated on the wells of a microtiter plate is pre-incubated with or without candidate competing antibody and then the biotin labeled anti-CCR8 antibody of the invention is added. The amount of labeled anti-CCR8 antibody bound to the CCR8 antigen in the wells is measured using avidin-peroxidase conjugate and appropriate substrate. The antibody can be labeled with a radioactive or fluorescent label or some other detectable and measurable label. The amount of labeled anti-CCR8 antibody that bound to the antigen will have an indirect correlation to the ability of the candidate competing antibody (test antibody) to compete for binding to the same epitope, i.e., the greater the affinity of the test antibody for the same epitope, the less labeled antibody will be bound to the antigen-coated wells. A candidate competing antibody is considered an antibody that binds substantially to the same epitope or that competes for binding to the same epitope as an anti-CCR8 antibody of the invention if the candidate antibody can block binding of the CCR8 antibody by at least 20%, by at least 30%, by at least 40%, or by at least 50% as compared to the control performed in parallel in the absence of the candidate competing antibody. It will be understood that variations of this assay can be performed to arrive at the same quantitative value.

In certain alternative embodiments, the antibodies of the present invention can be engineered by modifying one or more residues within one or both variable regions (i.e., V_{H} and/or V_{L}), or by modifying residues within the constant region(s), e.g., to alter the effector function(s) of the antibody. In various embodiments, the variable region of the antibody will by modified by performing CDR grafting using framework sequences can be obtained from public DNA databases or published references that include germline antibody gene sequences (e.g., Tomlinson, I. M., et al., J. Mol. Biol. 227:776-798, 1992; and Cox, J. P. L. et al., Eur. J. Immunol. 24:827-836, 1994; the contents of each of which are expressly incorporated herein by reference). In various embodiments, the antibodies may be modified using site-directed mutagenesis or PCR-mediated mutagenesis to introduce a mutation(s) in the VH and/or VL which improves binding affinity and/or decreases immunogenicity. In various embodiments, the antibodies may be modified in the Fc region for purposes of altering the serum half-life, complement fixation, Fc receptor binding, and/or antigen-dependent cellular cytotoxicity of the antibody. In various embodiments, the antibodies may be modified for purposes of modifying the glycosylation of the antibody. Methods for performing each of the modifications described herein, and others, are well known to the skilled artisan.

### Pharmaceutical Compositions

In another aspect, the present invention provides a pharmaceutical composition comprising an antibody or antigen-binding fragment thereof as described above. The pharmaceutical compositions, methods and uses of the invention thus also encompass embodiments of combinations (co-administration) with other active agents, as detailed below.

Generally, the antibodies, or antigen-binding fragments thereof antibodies of the present invention are suitable to be administered as a formulation in association with one or more pharmaceutically acceptable excipient(s). The term ‘excipient’ is used herein to describe any ingredient other than the compound(s) of the invention. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody. Pharmaceutical compositions of the present invention and methods for their preparation will be readily apparent to those skilled in the art. Such compositions and methods for their preparation may be found, for example, in Remington's Pharmaceutical Sciences, 19th Edition (Mack Publishing Company, 1995). Pharmaceutical compositions are preferably manufactured under GMP conditions.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Any method for administering peptides, proteins or antibodies accepted in the art may suitably be employed for the antibodies and portions of the invention.

The pharmaceutical compositions of the invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. Various embodiments include the intravenous and the subcutaneous routes.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

For example, in one aspect, sterile injectable solutions can be prepared by incorporating the anti-CCR8 antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies of the invention can also be administered intranasally or by inhalation, typically in the form of a dry powder (either alone, as a mixture, or as a mixed component particle, for example, mixed with a suitable pharmaceutically acceptable excipient) from a dry powder inhaler, as an aerosol spray from a pressurized container, pump, spray, atomizer (preferably an atomizer using electrohydrodynamics to produce a fine mist), or nebulizer, with or without the use of a suitable propellant, or as nasal drops.

The pressurized container, pump, spray, atomizer, or nebulizer generally contains a solution or suspension of an antibody of the invention comprising, for example, a suitable agent for dispersing, solubilizing, or extending release of the active, a propellant(s) as solvent.

Prior to use in a dry powder or suspension formulation, the drug product is generally micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the compound of the invention, a suitable powder base and a performance modifier.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate- and/or modified release. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” of an antibody of the invention. The overall daily dose will typically be administered in a single dose or, more usually, as divided doses throughout the day.

The antibodies and antibody portions of the invention may also be formulated for an oral route administration. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Pharmaceutical compositions intended for oral use may be prepared according to any method known to the art for the manufacture of pharmaceutical compositions and such compositions may contain one or more agents selected from the group consisting of sweetening agents in order to provide a pharmaceutically elegant and palatable preparation. For example, to prepare orally deliverable tablets, the antibody or antigen-binding fragment thereof is mixed with at least one pharmaceutical excipient, and the solid formulation is compressed to form a tablet according to known methods, for delivery to the gastrointestinal tract. The tablet composition is typically formulated with additives, e.g. a saccharide or cellulose carrier, a binder such as starch paste or methyl cellulose, a filler, a disintegrator, or other additives typically usually used in the manufacture of medical preparations. To prepare orally deliverable capsules, DHEA is mixed with at least one pharmaceutical excipient, and the solid formulation is placed in a capsular container suitable for delivery to the gastrointestinal tract. Compositions comprising antibodies or antigen-binding fragments thereof may be prepared as described generally in Remington's Pharmaceutical Sciences, 18th Ed. 1990 (Mack Publishing Co. Easton Pa. 18042) at Chapter 89, which is herein incorporated by reference.

In various embodiments, the pharmaceutical compositions are formulated as orally deliverable tablets containing antibodies or antigen-binding fragments thereof in admixture with non-toxic pharmaceutically acceptable excipients which are suitable for manufacture of tablets. These excipients may be inert diluents, such as calcium carbonate, sodium carbonate, lactose, calcium phosphate or sodium phosphate; granulating and disintegrating agents, for example, maize starch, gelatin or acacia, and lubricating agents, for example, magnesium stearate, stearic acid, or talc. The tablets may be uncoated or they may be coated with known techniques to delay disintegration and absorption in the gastrointestinal track and thereby provide a sustained action over a longer period of time. For example, a time delay material such as glyceryl monostearate or glyceryl distearate alone or with a wax may be employed.

In various embodiments, the pharmaceutical compositions are formulated as hard gelatin capsules wherein the antibody or antigen-binding fragment thereof is mixed with an inert solid diluent, for example, calcium carbonate, calcium phosphate, or kaolin or as soft gelatin capsules wherein the antibody or antigen-binding fragment thereof is mixed with an aqueous or an oil medium, for example, arachis oil, peanut oil, liquid paraffin or olive oil.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

### Therapeutic Uses

In another aspect, the present invention relates to methods of treating a subject suffering from a CCR8-associated disorder, comprising administering to said subject a therapeutically effective amount of an antibody or antigen-binding fragment thereof of the present invention. In various embodiments, the subject is a human subject. In various embodiments, the CCR8-associated disorder is a cancer. In various embodiments, the cancerous cell is selected from the group consisting of ovarian cancer, lung cancer, breast cancer, gastric cancer, prostate cancer, colorectal cancer, renal cell cancer, liver cancer, pancreatic cancer, glioblastoma, melanoma and sarcoma. In various embodiments, the subject previously responded to treatment with an anti-cancer therapy, but, upon cessation of therapy, suffered relapse (hereinafter “a recurrent cancer”). In various embodiments, the subject has resistant or refractory cancer. In various embodiments, the cancerous cells are immunogenic tumors (e.g., those tumors for which vaccination using the tumor itself can lead to immunity to tumor challenge).

In various embodiments, a method for treating a subject afflicted with a cancer comprises administering to the subject a therapeutically effective amount of any one of the Treg-depleting anti-CCR8 Abs, e.g., mAbs, immunoconjugates or bispecific molecules disclosed herein, or a pharmaceutical composition comprising any one of said Abs, e.g., anti-CCR8 mAbs, immunoconjugates or bispecific molecules, such that the subject is treated.

In another aspect, the present invention relates to combination therapies designed to treat a cancer in a subject. In various embodiments, a method for inhibiting growth of tumor cells in a subject comprises administering to the subject a therapeutically effective amount of: (a) any one of the Treg-depleting anti-CCR8 Abs, immunoconjugates or bispecific molecules disclosed herein, or a pharmaceutical composition comprising any one of said anti-CCR8 Abs, immunoconjugates or bispecific molecules; and (b) an additional therapy for treating cancer. In various embodiments, the additional therapeutic therapy is a therapeutic agent that is a compound that reduces inhibition, or increases stimulation, of the immune system, such that growth of tumor cells in the subject is inhibited. In various embodiments, the additional therapy is selected from the group consisting of immunotherapy, chemotherapy, small molecule kinase inhibitor targeted therapy, surgery, radiation therapy, and stem cell transplantation, wherein the combination therapy provides increased cell killing of tumor cells, i.e., a synergy exists between the isolated antibody or antigen-binding fragment and the additional therapies when co-administered.

In another aspect, the present invention relates to methods for enhancing the immune response to cancerous cells in a subject, comprising administering to the subject a therapeutically effective amount (either as monotherapy or in a combination therapy regimen) of an isolated antibody or antigen-binding fragment of the present invention. In various embodiments, the present invention provides for a method of treating cancerous cells in a subject, comprising administering to said subject a therapeutically effective amount (either as monotherapy or in a combination therapy regimen) of an antibody or antigen-binding fragment thereof of the present invention. In various embodiments, the cancerous cell is selected from the group consisting of ovarian cancer, lung cancer, breast cancer, gastric cancer, prostate cancer, colorectal cancer, renal cell cancer, liver cancer, pancreatic cancer, glioblastoma, melanoma and sarcoma.

In various embodiments, the cancer to be treated includes, but is not limited to solid tumors, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, and combinations of said cancers. In various embodiments, the cancerous cells are immunogenic tumors (e.g., those tumors for which vaccination using the tumor itself can lead to immunity to tumor challenge). In various embodiments, the cancer is selected from the group consisting of melanoma (e.g., metastatic malignant melanoma), colorectal cancer (CRC), renal cancer, bladder cancer, non-small-cell lung cancer (NSCLC), prostate cancer, breast cancer, colon cancer, ovarian cancer and lung cancer.

In various embodiments the solid tumor is a cancer chosen from HNSCC, cervical, CRC, NSCLC-SCC, NSCLC-ADC, pancreatic, gastric, bladder, and breast cancers.

In various embodiments, the cancer is a hematological malignancy which include liquid tumors derived from either of the two major blood cell lineages, i.e., the myeloid cell line (which produces granulocytes, erythrocytes, thrombocytes, macrophages and mast cells) or the lymphoid cell line (which produces B, T, NK and plasma cells), including all types of leukemias, lymphomas, and myelomas. Hematological malignancies that may be treated using the present therapy methods include, for example, cancers selected from acute lymphoblastic leukemia (ALL), acute myelogenous leukemia (AML), chronic lymphocytic leukemia (CLL), chronic myelogenous leukemia (CML), Hodgkin's lymphoma (HL), non-Hodgkin's lymphomas (NHLs), multiple myeloma, smoldering myeloma, monoclonal gammopathy of undetermined significance (MGUS), advanced, metastatic, refractory and/or recurrent hematological malignancies, and any combinations of said hematological malignancies.

In various embodiments, the present antibodies and antigen-binding fragments thereof can be utilized to directly kill or ablate cancerous cells in vivo. Direct killing involves administering the antibodies (which are optionally fused to a cytotoxic drug) to a subject requiring such treatment. In various embodiments, the cancer comprises cancer cells expressing CCR8 at a higher level than noncancerous cells of a comparable tissue. Since the antibodies recognize CCR8 on cancer cells, any such cells to which the antibodies bind are destroyed. Where the antibodies are used alone to kill or ablate cancer cells, such killing or ablation can be affected by initiating endogenous host immune functions, such as CDC and/or ADCC. Assays for determining whether an antibody kills cells in this manner are within the purview of those skilled in the art.

In various embodiments, the present antibodies and antigen-binding fragments thereof can be utilized to promote growth inhibition and/or proliferation of a cancerous tumor cell. These methods may inhibit or prevent the growth of the cancer cells of said subject, such as for example, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. As a result, where the cancer is a solid tumor, the modulation may reduce the size of the solid tumor by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

The inhibition of the cancer cell proliferation can be measured by cell-based assays, such as bromodeoxyuridine (BRDU) incorporation (Hoshino et al., Int. J. Cancer 38, 369, 1986; Campana et al., J. Immunol. Meth. 107:79, 1988; [3H]-thymidine incorporation (Chen, J., Oncogene 13:1395-403, 1996; Jeoung, J., J. Biol. Chem. 270:18367-73, 1995; the dye Alamar Blue (available from Biosource International) (Voytik-Harbin et al., In Vitro Cell Dev Biol Anim 34:239-46, 1998). The anchorage independent growth of cancer cells is assessed by colony formation assay in soft agar, such as by counting the number of cancer cell colonies formed on top of the soft agar (see Examples and Sambrook et al., Molecular Cloning, Cold Spring Harbor, 1989).

The inhibition of cancer cell growth in a subject may be assessed by monitoring the cancer growth in a subject, for example in an animal model or in human subjects. One exemplary monitoring method is tumorigenicity assays. In one example, a xenograft comprises human cells from a pre-existing tumor or from a tumor cell line. Tumor xenograft assays are known in the art and described herein (see, e.g., Ogawa et al., Oncogene 19:6043-6052, 2000). In another embodiment, tumorigenicity is monitored using the hollow fiber assay, which is described in U.S. Pat. No. 5,698,413, which is incorporated herein by reference in its entirety.

The percentage of the inhibition is calculated by comparing the cancer cell proliferation, anchorage independent growth, or cancer cell growth under modulator treatment with that under negative control condition (typically without modulator treatment). For example, where the number of cancer cells or cancer cell colonies (colony formation assay), or PRDU or [3H]-thymidine incorporation is A (under the treatment of modulators) and C (under negative control condition), the percentage of inhibition would be (C−A)/C×100%.

Examples of tumor cell lines derived from human tumors and available for use in the in vitro and in vivo studies include, but are not limited to, leukemia cell lines (e.g., CCRF-CEM, HL-60 (TB), K-562, MOLT-4, RPM1-8226, SR, P388 and P388/ADR); non-small cell lung cancer cell lines (e.g., A549/ATCC, EKVX, HOP-62, HOP-92, NCI-H226, NCI-H23, NCI-H322M, NCI-H460, NCI-H522 and LXFL 529); small cell lung cancer cell lines (e.g., DMS 114 and SHP-77); colon cancer cell lines (e.g., COLO 205, HCC-2998, HCT-116, HCT-15, HT29, KM12, SW-620, DLD-1 and KM20L2); central nervous system (CNS) cancer cell lines (e.g., SF-268, SF-295, SF-539, SNB-19, SNB-75, U251, SNB-78 and XF 498); melanoma cell lines (e.g., LOX I MVI, MALME-3M, M14, SK-MEL-2, SK-MEL-28, SK-MEL-5, UACC-257, UACC-62, RPMI-7951 and M19-MEL); ovarian cancer cell lines (e.g., IGROV1, OVCAR-3, OVCAR-4, OVCAR-5, OVCAR-8 and SK-OV-3); renal cancer cell lines (e.g., 786-0, A498, ACHN, CAKI-1, RXF 393, SN12C, TK-10, UO-31, RXF-631 and SN12K1); prostate cancer cell lines (e.g., PC-3 and DU-145); breast cancer cell lines (e.g., MCF7, NCI/ADR-RES, MDA-MB-231/ATCC, HS 578T, MDA-MB-435, BT-549, T-47D and MDA-MB-468); and thyroid cancer cell lines (e.g., SK-N-SH).

“Therapeutically effective amount” or “therapeutically effective dose” refers to that amount of the therapeutic agent being administered which will relieve to some extent one or more of the symptoms of the disorder being treated.

A therapeutically effective dose can be estimated initially from cell culture assays by determining an IC₅₀. A dose can then be formulated in animal models to achieve a circulating plasma concentration range that includes the IC₅₀ as determined in cell culture. Such information can be used to more accurately determine useful doses in humans. Levels in plasma may be measured, for example, by HPLC. The exact composition, route of administration and dosage can be chosen by the individual physician in view of the subject's condition.

Dosage regimens can be adjusted to provide the optimum desired response (e.g., a therapeutic or prophylactic response). For example, a single bolus can be administered, several divided doses (multiple or repeat or maintenance) can be administered over time and the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure will be dictated primarily by the unique characteristics of the antibody and the particular therapeutic or prophylactic effect to be achieved.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

For administration to human subjects, the total monthly dose of the antibodies or antigen-binding fragments thereof of the disclosure can be in the range of 0.5-1200 mg per subject, 0.5-1100 mg per subject, 0.5-1000 mg per subject, 0.5-900 mg per subject, 0.5-800 mg per subject, 0.5-700 mg per subject, 0.5-600 mg per subject, 0.5-500 mg per subject, 0.5-400 mg per subject, 0.5-300 mg per subject, 0.5-200 mg per subject, 0.5-100 mg per subject, 0.5-50 mg per subject, 1-1200 mg per subject, 1-1100 mg per subject, 1-1000 mg per subject, 1-900 mg per subject, 1-800 mg per subject, 1-700 mg per subject, 1-600 mg per subject, 1-500 mg per subject, 1-400 mg per subject, 1-300 mg per subject, 1-200 mg per subject, 1-100 mg per subject, or 1-50 mg per subject depending, of course, on the mode of administration. For example, an intravenous monthly dose can require about 1-1000 mg/subject. In various embodiments, the antibodies or antigen-binding fragments thereof of the disclosure can be administered at about 1-200 mg per subject, 1-150 mg per subject or 1-100 mg/subject. The total monthly dose can be administered in single or divided doses and can, at the physician's discretion, fall outside of the typical ranges given herein.

In various embodiments, a non-limiting daily dosing range for a therapeutically or prophylactically effective amount of an antibody or antigen-binding fragment thereof of the disclosure can be 0.001 to 100 mg/kg, 0.001 to 90 mg/kg, 0.001 to 80 mg/kg, 0.001 to 70 mg/kg, 0.001 to 60 mg/kg, 0.001 to 50 mg/kg, 0.001 to 40 mg/kg, 0.001 to 30 mg/kg, 0.001 to 20 mg/kg, 0.001 to 10 mg/kg, 0.001 to 5 mg/kg, 0.001 to 4 mg/kg, 0.001 to 3 mg/kg, 0.001 to 2 mg/kg, 0.001 to 1 mg/kg, 0.010 to 50 mg/kg, 0.010 to 40 mg/kg, 0.010 to 30 mg/kg, 0.010 to 20 mg/kg, 0.010 to 10 mg/kg, 0.010 to 5 mg/kg, 0.010 to 4 mg/kg, 0.010 to 3 mg/kg, 0.010 to 2 mg/kg, 0.010 to 1 mg/kg, 0.1 to 50 mg/kg, 0.1 to 40 mg/kg, 0.1 to 30 mg/kg, 0.1 to 20 mg/kg, 0.1 to 10 mg/kg, 0.1 to 5 mg/kg, 0.1 to 4 mg/kg, 0.1 to 3 mg/kg, 0.1 to 2 mg/kg, 0.1 to 1 mg/kg, 1 to 50 mg/kg, 1 to 40 mg/kg, 1 to 30 mg/kg, 1 to 20 mg/kg, 1 to 10 mg/kg, 1 to 5 mg/kg, 1 to 4 mg/kg, 1 to 3 mg/kg, 1 to 2 mg/kg, or 1 to 1 mg/kg body weight.

For repeated administrations over several days or longer, depending on the condition, the treatment is sustained until a desired suppression of symptoms occurs or until sufficient therapeutic levels are achieved, for example, to reduce pain. An exemplary dosing regimen comprises administering an initial dose of about 2 mg/kg, followed by a weekly maintenance dose of about 1 mg/kg of the anti-CCR8 antibody, or followed by a maintenance dose of about 1 mg/kg every other week. However, other dosage regimens may be useful, depending on the pattern of pharmacokinetic decay that the practitioner wishes to achieve. For example, in some embodiments, dosing from one-four times a week is contemplated. The progress of this therapy is easily monitored by conventional techniques and assays. The dosing regimen (including the CCR8 antagonist(s) used) can vary over time. In various embodiments, the appropriate dosage of an anti-CCR8 antagonist antibody will depend on the anti-CCR8 antagonist antibody (or compositions thereof) employed, the type and severity of headache (e.g., migraine) to be treated, whether the agent is administered for preventive or therapeutic purposes, previous therapy, the patient's clinical history and response to the agent, and the discretion of the attending physician. Typically, the clinician will administer an anti-CCR8 antagonist antibody, until a dosage is reached that achieves the desired result. Dose and/or frequency can vary over course of treatment.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In various embodiments, the total dose administered will achieve a plasma antibody concentration in the range of, e.g., about 1 to 1000 μg/ml, about 1 to 750 μg/ml, about 1 to 500 μg/ml, about 1 to 250 μg/ml, about 10 to 1000 μg/ml, about 10 to 750 μg/ml, about 10 to 500 μg/ml, about 10 to 250 μg/ml, about 20 to 1000 μg/ml, about 20 to 750 μg/ml, about 20 to 500 μg/ml, about 20 to 250 μg/ml, about 30 to 1000 μg/ml, about 30 to 750 μg/ml, about 30 to 500 μg/ml, about 30 to 250 μg/ml.

Toxicity and therapeutic index of the pharmaceutical compositions of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effective dose is the therapeutic index and it can be expressed as the ratio LD₅₀/ED₅₀. Compositions that exhibit large therapeutic indices are generally preferred.

In various embodiments, single or multiple administrations of the pharmaceutical compositions are administered depending on the dosage and frequency as required and tolerated by the subject. In any event, the composition should provide a sufficient quantity of at least one of the antibodies or antigen-binding fragments thereof disclosed herein to effectively treat the subject. The dosage can be administered once but may be applied periodically until either a therapeutic result is achieved or until side effects warrant discontinuation of therapy.

The dosing frequency of the administration of the antibody or antigen-binding fragment thereof pharmaceutical composition depends on the nature of the therapy and the particular disease being treated. The subject can be treated at regular intervals, such as weekly or monthly, until a desired therapeutic result is achieved. Exemplary dosing frequencies include, but are not limited to: once weekly without break; once weekly, every other week; once every 2 weeks; once every 3 weeks; weakly without break for 2 weeks, then monthly; weakly without break for 3 weeks, then monthly; monthly; once every other month; once every three months; once every four months; once every five months; or once every six months, or yearly.

### Combination Therapy

As used herein, the terms “co-administration”, “co-administered” and “in combination with”, referring to the antibodies or antigen-binding fragments thereof of the disclosure and one or more other therapeutic agents, is intended to mean, and does refer to and include the following: simultaneous administration of such combination of antibodies or antigen-binding fragments thereof of the disclosure and therapeutic agent(s) to a subject in need of treatment, when such components are formulated together into a single dosage form which releases said components at substantially the same time to said subject; substantially simultaneous administration of such combination of antibodies or antigen-binding fragments thereof of the disclosure and therapeutic agent(s) to a subject in need of treatment, when such components are formulated apart from each other into separate dosage forms which are taken at substantially the same time by said subject, whereupon said components are released at substantially the same time to said subject; sequential administration of such combination of antibodies or antigen-binding fragments thereof of the disclosure and therapeutic agent(s) to a subject in need of treatment, when such components are formulated apart from each other into separate dosage forms which are taken at consecutive times by said subject with a significant time interval between each administration, whereupon said components are released at substantially different times to said subject; and sequential administration of such combination of antibodies or antigen-binding fragments thereof of the disclosure and therapeutic agent(s) to a subject in need of treatment, when such components are formulated together into a single dosage form which releases said components in a controlled manner whereupon they are concurrently, consecutively, and/or overlappingly released at the same and/or different times to said subject, where each part may be administered by either the same or a different route.

In another aspect, the present invention relates to combination therapies designed to treat a cancer, or an infectious disease, in an subject, comprising administering to the subject a therapeutically effective amount of an isolated antibody or antigen-binding fragment of the present invention, and b) one or more additional therapies selected from the group consisting of immunotherapy, chemotherapy, small molecule kinase inhibitor targeted therapy, surgery, radiation therapy, vaccination protocols, and stem cell transplantation, wherein the combination therapy provides increased cell killing of tumor cells, i.e., a synergy exists between the isolated antibody or antigen-binding fragment and the additional therapies when co-administered.

In various embodiments, the immunotherapy is selected from the group consisting of: treatment using agonistic, antagonistic, or blocking antibodies to co-stimulatory or co-inhibitory molecules (immune checkpoints) such as PD-1, PD-L1, OX-40, CD137, GITR, LAG3, TIM-3, CD40, TIGIT, CD47, SIRPa and VISTA; treatment using bispecific T cell engaging antibodies (BITE®) such as blinatumomab: treatment involving administration of biological response modifiers such as IL-2, IL-7, IL-12, IL-15, IL-21, GM-CSF, STING agonists, and IFN-IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

In various embodiments, the additional therapy comprises an antibody that specifically binds an immune-checkpoint protein antigen from the list including, but not limited to, CD276, CD272, CD152, CD223, CD279, CD274, TIM-3 and B7-H4; or any immune-checkpoint protein antigen antibody taught in the art. In various embodiments, the PD-1 inhibitor used in the combination therapy methods is selected from the group consisting of, but not limited to, pembrolizumab (Merck), nivolumab (Bristol-Myers Squibb), cemiplimab (Regeneron), dostarlimab (GlaxoSmithKline), and retifanlimab (Incyte). In various embodiments, the PD-1 inhibitor is pembrolizumab. In various embodiments, the PD-1 inhibitor is nivolumab. In various embodiments, the PD-1 inhibitor is cemiplimab. In various embodiments, the PD-1 inhibitor is dostarlimab. In various embodiments, the PD-1 inhibitor is retifanlimab. In various embodiments, between about 0.1 mg/kg to about 10 mg/kg of PD-1 inhibitor is administered. In various embodiments, between about 1 mg/kg to about 15 mg/kg of PD-1 inhibitor is administered.

A wide array of conventional compounds has been shown to have anti-neoplastic activities. These compounds have been used as pharmaceutical agents in chemotherapy to shrink solid tumors, prevent metastases and further growth, or decrease the number of malignant T-cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments

When the antibody or antigen-binding fragment disclosed herein is administered in combination with another conventional anti-neoplastic agent, either concomitantly or sequentially, such antibody or antigen-binding fragment may enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant T-cells.

Pharmaceutical compounds that may be used for combinatory anti-tumor therapy include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments, the chemotherapy comprises a chemotherapeutic agent selected from the group consisting of: daunorubicin, dactinomycin, doxorubicin, bleomycin, mitomycin, nitrogen mustard, chlorambucil, melphalan, cyclophosphamide, 6-mercaptopurine, 6-thioguanine, bendamustine, cytarabine (CA), 5-fluorouracil (5-FU), floxuridine (5-FUdR), methotrexate (MTX), colchicine, vincristine, vinblastine, etoposide, teniposide, cisplatin, carboplatin, oxaliplatin, pentostatin, cladribine, cytarabine, gemcitabine, pralatrexate, mitoxantrone, diethylstilbestrol (DES), fluradabine, ifosfamide, hydroxyureataxanes (such as paclitaxel and doxetaxel) and/or anthracycline antibiotics, as well as combinations of agents such as, but not limited to, DA-EPOCH, CHOP, CVP or FOLFOX.

In various embodiments, the small molecule kinase inhibitor targeted therapy comprises a small molecule kinase inhibitor selected from the group consisting of Bruton's tyrosine kinase (BTK) inhibitor, phosphatidylinositol-3-kinase (PI3K) inhibitor, SYK inhibitor (e.g., entospletinib), AKT inhibitor, mTOR inhibitor, Src inhibitor, JAK/STAT inhibitor, Ras/Raf/MEK/ERK inhibitor, and Aurora inhibitor (see, D'Cruz et al, Expert Opin Pharmacother, 14 (6): 707-21, 2013).

In various embodiments, the combination therapy comprises administering the antibody or antigen-binding fragment thereof and the one or more additional therapies simultaneously. In various embodiments, antibody or antigen-binding fragment thereof composition and the one or more additional therapies are administered sequentially, i.e., the antibody or antigen-binding fragment thereof composition is administered either prior to or after the administration of the one or more additional therapies.

In various embodiments, the administrations of the antibody or antigen-binding fragment thereof composition and the one or more additional therapies are concurrent, i.e., the administration period of the antibody or antigen-binding fragment thereof composition and the one or more additional therapies overlap with each other.

In various embodiments, the administrations of the antibody or antigen-binding fragment thereof composition and the one or more additional therapies are non-concurrent. For example, in various embodiments, the administration of the antibody or antigen-binding fragment thereof composition is terminated before the one or more additional therapies is administered. In various embodiments, the administration of the one or more additional therapies is terminated before the antibody or antigen-binding fragment thereof composition is administered.

When the antibody or antigen-binding fragment thereof disclosed herein is administered in combination with one or more additional therapies, either concomitantly or sequentially, such antibody or antigen-binding fragment thereof may enhance the therapeutic effect of the one or more additional therapies or overcome cellular resistance to the one or more additional therapies. This allows for decreased dosage or duration of the one or more additional therapies, thereby reducing the undesirable side effects, or restores the effectiveness of the one or more additional therapies.

### Diagnostic Uses

In another aspect, the present invention provides a method for detecting in vitro or in vivo the presence of human CCR8 peptide in a sample, e.g., for diagnosing a human CCR8-related disorder. In some methods, this is achieved by contacting a sample to be tested, along with a control sample, with a human sequence antibody or a human monoclonal antibody of the invention, or an antigen-binding portion thereof (or a bispecific or multispecific molecule), under conditions that allow for formation of a complex between the antibody and human CCR8. Complex formation is then detected (e.g., using an ELISA) in both samples, and any statistically significant difference in the formation of complexes between the samples is indicative the presence of human CCR8 antigen in the test sample.

In various embodiments, methods are provided for detecting a CCR8-related disorder or confirming the diagnosis of a CCR8-related disorder in a subject. The method includes contacting a biological sample from the subject with an isolated antibody or antigen-biding fragment thereof of the invention and detecting binding of the isolated human monoclonal antibody or antigen-binding fragment thereof to the sample. An increase in binding of the isolated human monoclonal antibody or antigen-binding fragment thereof to the sample as compared to binding of the isolated human monoclonal antibody or antigen-binding fragment thereof to a control sample detects a CCR8-related disorder in the subject or confirms the diagnosis of a CCR8-related disorder in the subject. The control can be a sample from a subject known not to have a CCR8-related disorder, or a standard value. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, and spinal fluid.

In one embodiment, a kit is provided for detecting CCR8 in a biological sample, such as a blood sample. Kits for detecting a polypeptide will typically comprise a human antibody that specifically binds CCR8, such as any of the antibodies disclosed herein. In some embodiments, an antibody fragment, such as an Fv fragment is included in the kit. For in vivo uses, the antibody can be a scFv fragment. In a further embodiment, the antibody is labeled (for example, with a fluorescent, radioactive, or an enzymatic label).

In one embodiment, a kit includes instructional materials disclosing means of use of an antibody that specifically binds CCR8. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment, the diagnostic kit comprises an immunoassay. Although the details of the immunoassays may vary with the particular format employed, the method of detecting CCR8 in a biological sample generally includes the steps of contacting the biological sample with an antibody which specifically reacts, under immunologically reactive conditions, to CCR8. The antibody is allowed to specifically bind under immunologically reactive conditions to form an immune complex, and the presence of the immune complex (bound antibody) is detected directly or indirectly.

In various embodiments, the antibodies or antigen-binding fragments can be labeled or unlabeled for diagnostic purposes. Typically, diagnostic assays entail detecting the formation of a complex resulting from the binding of an antibody to CCR8. The antibodies can be directly labeled. A variety of labels can be employed, including, but not limited to, radionuclides, fluorescers, enzymes, enzyme substrates, enzyme cofactors, enzyme inhibitors and ligands (e.g., biotin, haptens). Numerous appropriate immunoassays are known to the skilled artisan (see, for example, U.S. Pat. Nos. 3,817,827; 3,850,752; 3,901,654; and 4,098,876). When unlabeled, the antibodies can be used in assays, such as agglutination assays. Unlabeled antibodies can also be used in combination with another (one or more) suitable reagent which can be used to detect antibody, such as a labeled antibody (e.g., a second antibody) reactive with the first antibody (e.g., anti-idiotype antibodies or other antibodies that are specific for the unlabeled immunoglobulin) or other suitable reagent (e.g., labeled protein A).

The antibody or antigen-binding fragment provided herein may also be used in a method of detecting the susceptibility of a mammal to certain diseases. To illustrate, the method can be used to detect the susceptibility of a mammal to diseases which progress based on the amount of CCR8 present on cells and/or the number of CCR8-positive cells in a mammal. In one embodiment, the application provides a method of detecting susceptibility of a mammal to a tumor. In this embodiment, a sample to be tested is contacted with an antibody which binds to CCR8 or portion thereof under conditions appropriate for binding of said antibody thereto, wherein the sample comprises cells which express CCR8 in normal individuals. The binding of antibody and/or amount of binding is detected, which indicates the susceptibility of the individual to a tumor, wherein higher levels of receptor correlate with increased susceptibility of the individual to a tumor.

In various embodiments, the antibodies or antigen-binding fragments are attached to a label that is able to be detected (e.g., the label can be a radioisotope, fluorescent compound, enzyme or enzyme co-factor). The active moiety may be a radioactive agent, such as: radioactive heavy metals such as iron chelates, radioactive chelates of gadolinium or manganese, positron emitters of oxygen, nitrogen, iron, carbon, or gallium, ⁴³K, ⁵²Fe, ⁵⁷Co, ⁶⁷Cu, ⁶⁷Ga, ⁶⁸Ga, ¹²³I, ¹²⁵I, ¹³¹I, ¹³²I, or ⁹⁹Tc. A binding agent affixed to such a moiety may be used as an imaging agent and is administered in an amount effective for diagnostic use in a mammal such as a human and the localization and accumulation of the imaging agent is then detected. The localization and accumulation of the imaging agent may be detected by radioscintigraphy, nuclear magnetic resonance imaging, computed tomography or positron emission tomography.

Immunoscintigraphy using antibodies or antigen-binding fragments directed at CCR8 may be used to detect and/or diagnose cancers and vasculature. For example, monoclonal antibodies against the CCR8 marker labeled with ⁹⁹Technetium, ¹¹¹Indium, or ¹²⁵Iodine may be effectively used for such imaging. As will be evident to the skilled artisan, the amount of radioisotope to be administered is dependent upon the radioisotope. Those having ordinary skill in the art can readily formulate the amount of the imaging agent to be administered based upon the specific activity and energy of a given radionuclide used as the active moiety. Typically, 0.1-100 millicuries per dose of imaging agent, or 1-10 millicuries, or 2-5 millicuries are administered. Thus, the compositions disclosed are useful as imaging agents comprising a targeting moiety conjugated to a radioactive moiety comprise 0.1-100 millicuries, in some embodiments 1-10 millicuries, in some embodiments 2-5 millicuries, in some embodiments 1-5 millicuries.

### Antibody-Drug Conjugates (ADCs) and Immunoconjugates

Antibody-drug conjugates (ADCs) combine the binding specificity of an antibody with the potency of drugs such as, for example, cytotoxic agents, anticancer and immunosuppressive drugs. The use of ADCs allows the target-specific delivery of drugs which, if administered as unconjugated drugs, may result in unacceptable levels of toxicity to normal cells. The mechanism of an ADC is to recognize and bind to specific antigen through the antibodies, trigger a series of reactions, and then enter the cytoplasm through the endocytosis, where the highly cytotoxic drug is dissociated from the antibody after th degradation by lysosomal enzymes to kill cancer cells. Compared with the traditional chemotherapy which causes damage to both cancer cells and normal tissues indiscriminately, targeting drug delivery can make the drug act on cancer cells directly and reduce the damage to normal cells.

The application further provides ADCs comprising the novel antibodies and antigen-binding fragments of the present invention linked to a second molecule selected from the group consisting of a cytotoxic agent, anticancer drug, or immunosuppressive drugs.

The cytotoxic compounds contemplated for use in antibody-drug conjugates inhibit various essential cellular targets, such as microtubules (maytansinoids, auristatins, taxanes: U.S. Pat. Nos. 5,208,020; 5,416,064; 6,333,410; 6,441,163; 6,340,701: 6,372,738; 6,436,931; 6,596,757: 7,276,497; 7,301,019; 7,303,749; 7,368,565; 7,473,796; 7,585,857; 7,598,290: 7,495,114; 7,601,354, U.S. patents application Nos. 20100092495, 20100129314, 20090274713, 20090076263, 20080171865) and DNA (calicheamicin, doxorubicin, CC-1065 analogues: U.S. Pat. Nos. 5,475,092: 5,585,499; 5,846,545; 6,534,660; 6,756,397; 6,630,579; 7,388,026; 7,655,660; 7,655,661).

The application further provides immunoconjugates or fusion proteins comprising an antibody or antigen-binding fragment thereof of the present invention conjugated (or linked) directly or indirectly to an effector molecule. In this regard, the term “conjugated” or “linked” refers to making two polypeptides into one contiguous polypeptide molecule. The linkage can be either by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. In various embodiments, an antibody or antigen-binding fragment is joined to an effector molecule. In other embodiments, an antibody or antigen-binding fragment joined to an effector molecule is further joined to a lipid, a protein or peptide to increase its half-life in the body. Accordingly in various embodiments, the antibodies of the present disclosure may be used to deliver a variety of effector molecules.

The effector molecule can be a detectable label, an immunotoxin, cytokine, chemokine, therapeutic agent, or chemotherapeutic agent.

Specific, non-limiting examples of immunotoxins include, but are not limited to, abrin, ricin, Pseudomonas exotoxin (PE, such as PE35, PE37, PE38, and PE40), diphtheria toxin (DT), botulinum toxin, cholix toxin, or modified toxins thereof, or other toxic agents that directly or indirectly inhibit cell growth or kill cells.

A “cytokine” is class of proteins or peptides released by one cell population which act on another cell as intercellular mediators. Cytokines can act as an immune-modulating agent. Examples of cytokines include lymphokines, monokines, growth factors and traditional polypeptide hormones. Thus, embodiments may utilize an interferon (e.g., IFN-α, IFN-β, and IFN-γ); tumor necrosis factor super family (TNFSF) member; human growth hormone; thyroxine; insulin; proinsulin; relaxin; prorelaxin; follicle stimulating hormone (FSH); thyroid stimulating hormone (TSH); luteinizing hormone (LH); hepatic growth factor; prostaglandin, fibroblast growth factor; prolactin; placental lactogen, OB protein; TNF-α; TNF-β; integrin; thrombopoietin (TPO); a nerve growth factor such as NGF-β; platelet-growth factor; TGF-α; TGF-β; insulin-like growth factor-I and -II; erythropoietin (EPO); colony stimulating factors (CSFs) such as macrophage-CSF (M-CSF); granulocyte-macrophage-CSF (GM-CSF); and granulocyte-CSF (G-CSF); an interleukin (IL-1 to IL-36), kit-ligand or FLT-3, angiostatin, thrombospondin, or endostatin; immune checkpoint proteins including CTLA-4, PD-1, PD-L1, LAG-3, TIGIT and TIM-3 as well as several others (Sharpe et al., Nat Immunol, 8:239-45, 2007). These cytokines include proteins from natural sources or from recombinant cell culture and biologically active equivalents of the native sequence cytokines.

In various embodiments, the effector molecule is selected from the list provided in Table 4. Each associated reference is incorporated herein by reference for the purpose of identifying the referenced tumor markers.

TABLE 4
Illustrative Tumor Markers

| Marker | Reference |
|---|---|
| 5 alpha reductase | Delos et al. (1998) Int J Cancer, 75: 6 840-846 |
| α-fetoprotein | Esteban et al. (1996) Tumour Biol., 17(5): 299-305 |
| AM-1 | Harada et al. (1996) Tohoku J Exp Med., 180(3): 273-288 |
| APC | Dihlmannet al. (1997) Oncol Res., 9(3) 119-127 |
| APRIL | Sordat et al. (1998) J Exp Med., 188(6): 1185-1190 |
| BAGE | Boel et al. (1995) Immunity, 2: 167-175. |
| β-catenin | Hugh et al. (1999) Int J Cancer, 82(4): 504-11 |
| Bc12 | Koty et al. (1999) Lung Cancer, 23(2): 115-127 |
| bcr-abl (b3a2) | Verfaillie et al. (1996) Blood, 87(11): 4770-4779 |
| CA-125 (Mucin 16) | Bast et al. (1998) Int J Biol Markers, 13(4): 179-187 |
| CASP-8/FLICE | Mandruzzato et al. (1997)J Exp Med., 186(5): 785-793. |
| Cathepsins | Thomssen et al. (1995)Clin Cancer Res., 1(7): 741-746 |
| CD19 | Scheuermann et al. (1995) Leuk Lymphoma, 18(5-6): 385-397 |
| CD20 | Knox et al. (1996) Clin Cancer Res., 2(3): 457-470 |
| CD21, CD23 | Shubinsky et al. (1997) Leuk Lymphoma, 25(5-6): 521-530 |
| CD22, CD38 | French et al. (1995) Br J Cancer, 71(5): 986-994 |
| CD33 | Nakase et al. (1996) Am J Clin Pathol., 105(6): 761-768 |
| CD35 | Yamakawa et al. Cancer, 73(11): 2808-2817 |
| CD44 | Naot et al. (1997) Adv Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin Exp Immunol., 85(3): 418-423 |
| CD52 | Ginaldi et al. (1998) Leuk Res., 22(2): 185-191 |
| CD55 | Spendlove et al. (1999) Cancer Res., 59: 2282-2286. |
| CD59 | Jarvis et al. (1997) Int J Cancer, 71(6): 1049-1055 |
| CDC27 | Wang et al. (1999) Science, 284(5418): 1351-1354 |
| CDK4 | Wolfel et al. (1995) Science, 269(5228): 1281-1284 |
| CEA | Kass et al. (1999) Cancer Res., 59(3): 676-683 |
| c-myc | Watson et al. (1991) Cancer Res., 51(15): 3996-4000 |
| Cox-2 | Tsujii et al. (1998) Cell, 93: 705-716 |
| DCC | Gotley et al. (1996) Oncogene, 13(4): 787-795 |
| DcR3 | Pitti et al. (1998) Nature, 396: 699-703 |
| E6/E7 | Steller et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) J. Clin. Invest., 102: 465-472. |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al (1992) J Biol Chem., 267(33): 24140-72414 |
| G250 | Divgi et al. (1998) Clin Cancer Res., 4(11): 2729-2739 |
| GAGE-Family | De Backer et al. (1999) Cancer Res., 59(13): 3157-3165 |
| gastrin 17 | Watson et al. (1995) Int J Cancer, 61(2): 233-240 |
| Gastrin-releasing | Wang et al. (1996) Int J Cancer, 68(4): 528-534 |
| hormone (bombesin) | |
| GD2/GD3/GM2 | Wiesner and Sweeley (1995) Int J Cancer, 60(3): 294-299 |
| GnRH | Bahk et al. (1998) Urol Res., 26(4): 259-264 |
| GnTV | Hengstler et al. (1998) Recent Results Cancer Res., 154: 47-85 |
| gp100/Pmel17 | Wagner et al. (1997) Cancer Immunol Immunther. 44(4): 239-247 |
| gp-100-in4 | Kirkin et al. (1998) APMIS, 106(7): 665-679 |
| gp15 | Maeurer et al. (1996) Melanoma Res., 6(1): 11-24 |
| gp75/TRP-1 | Lewis et al. (1995) Semin Cancer Biol., 6(6): 321-327 |
| hCG | Hoermann et al. (1992) Cancer Res., 52(6): 1520-1524 |
| Heparanase | Vlodavsky et al. (1999) Nat Med., 5(7): 793-802 |
| Her2/neu | Lewis et al. (1995) Semin Cancer Biol., 6(6): 321-327 |
| Her3 | |
| HMTV | Kahl et al. (1991)Br J Cancer, 63(4): 534-540 |
| Hsp70 | Jaattela et al. (1998) EMBO J., 17(21): 6124-6134 |
| hTERT | Vonderheide et al. (1999) Immunity, 10: 673-679. 1999. |
| (telomerase) | |
| IGFR1 | Ellis et al. (1998) Breast Cancer Res. Treat., 52: 175-184 |
| IL-13R | Murata et al. (1997) BiochemBiophysRes Commun., 238(1): 90-94 |
| iNOS | Klotz et al. (1998) Cancer, 82(10): 1897-1903 |
| Ki 67 | Gerdes et al. (1983) Int J Cancer, 31: 13-20 |
| KIAA0205 | Gueguen et al. (1998) J Immunol., 160(12): 6188-6194 |
| K-ras, H-ras, | Abrams et al. (1996) Semin Oncol., 23(1): 118-134 |
| N-ras | |
| KSA | Zhang et al. (1998) Clin Cancer Res., 4(2): 295-302 (CO17-1A) |
| LDLR-FUT | Caruso et al. (1998) Oncol Rep., 5(4): 927-930 |
| MAGE Family | Marchand et al. (1999) Int J Cancer, 80(2): 219-230 |
| (MAGE1, MAGE3, etc.) | |
| Mammaglobin | Watson et al. (1999) Cancer Res., 59: 13 3028-3031 |
| MAP17 | Kocher et al. (1996) Am J Pathol., 149(2): 493-500 |
| Melan-A/ | Lewis and Houghton (1995) Semin Cancer Biol., 6(6): 321-327 |
| MART-1 | |
| mesothelin | Chang et al. (1996)Proc. Natl. Acad. Sci., USA, 93(1): 136-140 |
| MIC A/B | Groh et al. (1998) Science, 279: 1737-1740 |
| MT-MMP's, such as | Sato and Seiki (1996) J Biochem (Tokyo), 119(2): 209-215 |
| MMP2, MMP3, MMP7, | |
| MMP9 | |
| Mox1 | Candia et al. (1992) Development, 116(4): 1123-1136 |
| Mucin, such as MUC-1, | Lewis and Houghton (1995) Semin Cancer Biol., 6(6): 321-327 |
| MUC-2, MUC-3, MUC-4 | |
| MUM-1 | Kirkin et al. (1998) APMIS, 106(7): 665-679 |
| NY-ESO-1 | Jager et al. (1998) J. Exp. Med., 187: 265-270 |
| Osteonectin | Graham et al. (1997) Eur J Cancer, 33(10): 1654-1660 |
| p15 | Yoshida et al. (1995) Cancer Res., 55(13): 2756-2760 |
| P170/MDR1 | Trock et al. (1997) J Natl Cancer Inst., 89(13): 917-931 |
| p53 | Roth et al. (1996) Proc. Natl. Acad. Sci., USA, 93(10): 4781-4786. |
| p97/melanotransferrin | Furukawa et al. (1989) J Exp Med., 169(2): 585-590 |
| PAI-1 | Grondahl-Hansen et al. (1993) Cancer Res., 53(11): 2513-2521 |
| PDGF | Vassbotn et al. (1993) Mol Cell Biol., 13(7): 4066-4076 |
| Plasminogen (uPA) | Naitoh et al. (1995) Jpn J Cancer Res., 86(1): 48-56 |
| PRAME | Kirkin et al. (1998) APMIS, 106(7): 665-679 |
| Probasin | Matuo et al. (1985) BiochemBiophysResComm., 130(1): 293-300 |
| Progenipoietin -- PSA | Sanda et al. (1999) Urology, 53(2): 260-266. |
| PSM | Kawakami et al. (1997) Cancer Res., 57(12): 2321-2324 |
| RAGE-1 | Gaugler et al. (1996) Immunogenetics, 44(5): 323-330 |
| Rb | Dosaka-Akita et al. (1997) Cancer, 79(7): 1329-1337 |
| RCAS1 | Sonoda et al. (1996) Cancer, 77(8): 1501-1509. |
| SART-1 | Kikuchi et al. (1999) Int J Cancer, 81(3): 459-466 |
| SSX gene | Gure et al. (1997) Int J Cancer, 72(6): 965-971 family |
| STAT3 | Bromberg et al. (1999) Cell, 98(3): 295-303 |
| STn | Sandmaier et al. (1999) J Immunother., 22(1): 54-66 |
| (mucin assoc.) | |
| TAG-72 | Kuroki et al. (1990) Cancer Res., 50(16): 4872-4879 |
| TGF-α | Imanishi et al. (1989) Br J Cancer, 59(5): 761-765 |
| TGF-β | Picon et al. (1998) CancerEpidemiolBiomarkerPrey, 7(6): 497-504 |
| Thymosin β 15 | Bao et al. (1996) Nature Medicine. 2(12), 1322-1328 |
| TNF-α | Moradi et al. (1993) Cancer, 72(8): 2433-2440 |
| TPA | Maulard et al. (1994) Cancer, 73(2): 394-398 |
| TPI | Nishida et al. (1984) Cancer Res 44(8): 3324-9 |
| TRP-2 | Parkhurst et al. (1998) Cancer Res., 58(21) 4895-4901 |
| Tyrosinase | Kirkin et al. (1998) APMIS, 106(7): 665-679 |
| VEGF | Hyodo et al. (1998) Eur J Cancer, 34(13): 2041-2045 |
| ZAG | Sanchez et al. (1999) Science, 283(5409): 1914-1919 |
| p16INK4 | Quelle et al. (1995) Oncogene Aug. 17, 1995; 11(4): 635-645 |
| Glutathione | Hengstler (1998) et al. Recent Results Cancer Res., 154: 47-85 |

In various embodiments, the effector molecule is selected from the list provided in Table 5. These targets can be applicable as cancer targeting as well.

TABLE 5
Targets for Autoimmune and inflammatory disorders or cancer

| Targets | RefSeq (protein) |
|---|---|
| IL-1 alpha | NP_000566 |
| IL-1 beta | NP_000567 |
| IL-2 | NP_000577 |
| IL-4 | NP_000580 |
| IL-4 induced 1 | NP_690863 |
| IL-5 | NP_000870 |
| IL-6 | NP_000591 |
| IL-6Rα | NP_000556 |
| IL-7 | NP_000871 |
| IL-10 | NP_000563 |
| IL-12 (alpha and beta) | NP_000873 and |
| IL-13 | NP_002179 |
| IL-17 | NP_002181 |
| IL-21 | NP_068575 |
| IL-22 | NP_065386 |
| IL-23 | NP_057668 |
| IL-33 | NP_254274 |
| TNF family (TNF- | NP_000585 |
| TNFR (TNFRSF1A) | NP_001056 |
| GMCSF | NP_000749 |
| IFN | NP_008831 |
| IFN alpha-beta | NP_000620 |
| APRIL | NP_003799 |
| Integrins (Integrin | NP_000880 |
| BAFF | NP_006564 |
| BAFFR | NP_443177 |
| CTLA4 | NP_005205 |
| BCR | NP_004318 |
| BLyS | NP_006564 |
| B7RP1 | NP_056074 |
| B7H1 | NP_054862 |
| B7H2 | NP_056074 |
| CXCR3 | NP_001495 |
| MCP1 | NP_002973 |
| BCMA | NP_001183 |
| TACI | NP_036584 |
| CD20 | NP_068769 |
| CD22 | NP_001762 |
| CD80 | NP_005182 |
| CD40 | NP_001241 |
| CD40L | NP_000065 |
| TSLP | NP_149024 |
| ICOS | NP_036224 |
| TLRs (TLR2 and | NP_003255 and |
| HMGB-1 | NP_002119 |
| HLA-DR | NP_001020330 |
| Collagen Type I | NP_000079 |
| Collagen Type II | NP_000080 |
| Fibronectin | XP_005246463 |
| Tenascin | NP_002151 |
| 1D10 | NP_114143 |

Chemokines can also be conjugated to the antibodies disclosed herein. Chemokines are a superfamily of small (approximately about 4 to about 14 K_{D}), inducible and secreted pro-inflammatory cytokines that act primarily as chemoattractants and activators of specific leukocyte cell subtypes. Chemokine production is induced by inflammatory cytokines, growth factors and pathogenic stimuli. The chemokine proteins are divided into subfamilies (alpha, beta, and delta) based on conserved amino acid sequence motifs and are classified into four highly conserved groups—CXC, CC, C and CX3C, based on the position of the first two cysteines that are adjacent to the amino terminus. To date, more than 50 chemokines have been discovered and there are at least 18 human seven-transmembrane-domain (7TM) chemokine receptors. Chemokines of use include, but are not limited to, RANTES, MCAF, MCP-1, and fractalkine.

The therapeutic agent can be a chemotherapeutic agent. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2.sup.nd ed., 2000 Churchill Livingstone, Inc; Baltzer L., Berkery R. (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). Useful chemotherapeutic agents for the preparation of immunoconjugates include auristatin, dolastatin, MMAE, MMAF, AFP, DM1, AEB, doxorubicin, daunorubicin, methotrexate, melphalan, chlorambucil, vinca alkaloids, 5-fluorouridine, mitomycin-C, taxol, L-asparaginase, mercaptopurine, thioguanine, hydroxyurea, cytarabine, cyclophosphamide, ifosfamide, nitrosoureas, cisplatin, carboplatin, mitomycin, dacarbazine, procarbazine, topotecan, nitrogen mustards, cytoxan, etoposide, BCNU, irinotecan, camptothecins, bleomycin, idarubicin, dactinomycin, plicamycin, mitoxantrone, asparaginase, vinblastine, vincristine, vinorelbine, paclitaxel, and docetaxel and salts, solvents and derivatives thereof. In various embodiments, the chemotherapeutic agent is auristatin E (also known in the art as dolastatin-10) or a derivative thereof as well as pharmaceutically salts or solvates thereof. Typical auristatin derivatives include DM1, AEB, AEVB, AFP, MMAF, and MMAE. The synthesis and structure of auristatin E and its derivatives, as well as linkers, are described in, e.g., U.S. Patent Application Publication No. 20030083263; U.S. Patent Application Publication No. 20050238629; and U.S. Pat. No. 6,884,869 (each of which is incorporated by reference herein in its entirety). In various embodiments, the therapeutic agent is an auristatin or an auristatin derivative. In various embodiments, the auristatin derivative is dovaline-valine-dolaisoleunine-dolaproine-phenylalanine (MMAF) or monomethyauristatin E (MMAE). In various embodiments, the therapeutic agent is a maytansinoid or a maytansinol analogue. In various embodiments, the maytansinoid is DM1.

The effector molecules can be linked to an antibody or antigen-binding fragment of the present invention using any number of means known to those of skill in the art. Both covalent and noncovalent attachment means may be used. The procedure for attaching an effector molecule to an antibody varies according to the chemical structure of the effector molecule. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of linker molecules such as those available from Pierce Chemical Company, Rockford, Ill. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site.

Procedures for conjugating the antibodies with the effector molecules have been previously described and are within the purview of one skilled in the art. For example, procedures for preparing enzymatically active polypeptides of the immunotoxins are described in WO84/03508 and WO85/03508, which are hereby incorporated by reference for purposes of their specific teachings thereof. Other techniques are described in Shih et al., Int. J. Cancer 41:832-839 (1988); Shih et al., Int. J. Cancer 46:1101-1106 (1990); Shih et al., U.S. Pat. No. 5,057,313; Shih Cancer Res. 51:4192, International Publication WO 02/088172; U.S. Pat. No. 6,884,869; International Patent Publication WO 2005/081711; U.S. Published Application 2003-0130189 A; and U.S. patent application No. 20080305044, each of which is incorporated by reference herein for the purpose of teaching such techniques.

An immunoconjugate of the present invention retains the immunoreactivity of the antibody or antigen-binding fragment, e.g., the antibody or antigen-binding fragment has approximately the same, or only slightly reduced, ability to bind the antigen after conjugation as before conjugation.

### Bispecific Molecules

A bispecific antibody is an antibody that includes two different antigen binding sites of monoclonal antibodies that can bind to two different antigens or two different sites of one antigen. In addition to simultaneously blocking two different signaling pathways and thereby enhancing tumor cell killing, bispecific antibody can also potentially increase binding specificity by interacting with two different cell-surface antigens instead of one. It is nowadays considered as a next generation of effective molecules for cancer therapy. It can minimize the regulatory and commercial issues that stem from administration of multiple therapeutic molecules. It also has the potential for novel activities that do not exist in mixtures of the parental antibodies. Several bispecific antibodies are marketed, and many are in clinical development.

In another aspect, the present invention features bispecific molecules comprising an anti-CCR8 antibody, or antigen-binding fragment thereof, of the invention. An antibody of the invention, or antigen-binding fragment thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results. In various embodiments, the invention includes bispecific molecules capable of binding both to FcγR or FcαR expressing effector cells (e.g., monocytes, macrophages or polymorphonuclear cells (PMNs)), and to target cells expressing PD. In such embodiments, the bispecific molecules target CCR8 expressing cells to effector cell and trigger Fc receptor-mediated effector cell activities, e.g., phagocytosis of an CCR8 expressing cells, antibody dependent cell-mediated cytotoxicity (ADCC), cytokine release, or generation of superoxide anion. Methods of preparing the bispecific molecules of the present invention are well known in the art.

In various embodiments, the second functional molecule is an antibody, antibody fragment, or protein or peptide that exhibit binding to an immune-checkpoint protein antigen that is present on the surface of an immune cell. In various embodiments, the immune-checkpoint protein antigen is selected from the group consisting of, but not limited to, CD276, CD272, CD152, CD223, CD279, CD274, CD40, SIRPa, CD47, OX-40, GITR, ICOS, CD27, 4-1BB, TIM-3, B7-H4, Siglec-7, Siglec-8, Siglec-9, Siglec-15, TIGIT, and VISTA. In various embodiments, D1 may comprise an antibody to an immune-checkpoint protein antigen is present on the surface of a tumor cell selected from the group consisting of, but are not limited to, PD-L1, B7-H3 and B7-H4.

In various embodiments of the present invention, the antibody or antigen-binding fragment is a CCR8/CTLA4 bispecific antibody comprising the heavy chain sequence of SEQ ID NO: 93:

(SEQ ID NO: 93)
EVQLVESGGGLVQPGGSLKLSCAASGFSFNTYAMNWVRQASGKGLEWVG
RIRTKSNNYATYYAASVKDRFTISRDDSKNTAYLQMNSLKTEDTAVYYC
TRGGSGLNYVRYFDVWGQGTTVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHN
HYTQKSLSLSPGKGGGGSGGGGSGGGGSEVQLVESGGGLVQPGGSLRLS
CAASGYTYSRHCLGWFRQAPGKGREAVSTIDSDGSTSYADSVKGRFTIS
RDNAKNTLYLQMNSLRPEDTAVYYCAIGPNPRYCSGAPNTRGAEHYFGY
WGQGTLVTVSS

and a light chain sequence selected from the group consisting of SEQ ID NO: 91 and SEQ ID NO: 92.

### Polynucleotides and Antibody Expression

The application further provides polynucleotides comprising a nucleotide sequence encoding an anti-CCR8 antibody or antigen-binding fragment thereof. Because of the degeneracy of the genetic code, a variety of nucleic acid sequences encode each antibody amino acid sequence. The application further provides polynucleotides that hybridize under stringent or lower stringency hybridization conditions, e.g., as defined herein, to polynucleotides that encode an antibody that binds to human CCR8.

Stringent hybridization conditions include, but are not limited to, hybridization to filter-bound DNA in 6×SSC at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR. In one embodiment, the codons that are used comprise those that are typical for human or mouse (see, e.g., Nakamura, Y., Nucleic Acids Res. 28:292 (2000)).

A polynucleotide encoding an antibody may also be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably polyA+RNA, isolated from, any tissue or cells expressing the antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

The present invention is also directed to host cells that express a CCR8 and/or the anti-CCR8 antibodies of the invention. A wide variety of host expression systems known in the art can be used to express an antibody of the present invention including prokaryotic (bacterial) and eukaryotic expression systems (such as yeast, baculovirus, plant, mammalian and other animal cells, transgenic animals, and hybridoma cells), as well as phage display expression systems.

An antibody of the invention can be prepared by recombinant expression of immunoglobulin light and heavy chain genes in a host cell. To express an antibody recombinantly, a host cell is transformed, transduced, infected or the like with one or more recombinant expression vectors carrying DNA fragments encoding the immunoglobulin light and/or heavy chains of the antibody such that the light and/or heavy chains are expressed in the host cell. The heavy chain and the light chain may be expressed independently from different promoters to which they are operably-linked in one vector or, alternatively, the heavy chain and the light chain may be expressed independently from different promoters to which they are operably-linked in two vectors one expressing the heavy chain and one expressing the light chain. Optionally, the heavy chain and light chain may be expressed in different host cells.

Additionally, the recombinant expression vector can encode a signal peptide that facilitates secretion of the antibody light and/or heavy chain from a host cell. The antibody light and/or heavy chain gene can be cloned into the vector such that the signal peptide is operably-linked in-frame to the amino terminus of the antibody chain gene. The signal peptide can be an immunoglobulin signal peptide or a heterologous signal peptide. Preferably, the recombinant antibodies are secreted into the medium in which the host cells are cultured, from which the antibodies can be recovered or purified.

An isolated DNA encoding a HCVR can be converted to a full-length heavy chain gene by operably linking the HCVR-encoding DNA to another DNA molecule encoding heavy chain constant regions. The sequences of human, as well as other mammalian, heavy chain constant region genes are known in the art. DNA fragments encompassing these regions can be obtained e.g., by standard PCR amplification. The heavy chain constant region can be of any type, (e.g., IgG, IgA, IgE, IgM or IgD), class (e.g., IgG₁, IgG₂, IgG₃ and IgG₄) or subclass constant region and any allotypic variant thereof as described in Kabat (supra).

An isolated DNA encoding a LCVR region may be converted to a full-length light chain gene (as well as to a Fab light chain gene) by operably linking the LCVR-encoding DNA to another DNA molecule encoding a light chain constant region. The sequences of human, as well as other mammalian, light chain constant region genes are known in the art. DNA fragments encompassing these regions can be obtained by standard PCR amplification. The light chain constant region can be a kappa or lambda constant region.

In addition to the antibody heavy and/or light chain gene(s), a recombinant expression vector of the invention carries regulatory sequences that control the expression of the antibody chain gene(s) in a host cell. The term “regulatory sequence” is intended to include promoters, enhancers and other expression control elements (e.g., polyadenylation signals), as needed, that control the transcription or translation of the antibody chain gene(s). The design of the expression vector, including the selection of regulatory sequences may depend on such factors as the choice of the host cell to be transformed, the level of expression of protein desired. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and/or polyoma virus.

Additionally, the recombinant expression vectors of the invention may carry additional sequences, such as sequences that regulate replication of the vector in host cells (e.g., origins of replication) and one or more selectable marker genes. The selectable marker gene facilitates selection of host cells into which the vector has been introduced. For example, typically the selectable marker gene confers resistance to drugs, such as G418, hygromycin, or methotrexate, on a host cell into which the vector has been introduced. Preferred selectable marker genes include the dihydrofolate reductase (dhfr) gene (for use in dhfr-minus host cells with methotrexate selection/amplification), the neo gene (for G418 selection), and glutamine synthetase (GS) in a GS-negative cell line (such as NSO) for selection/amplification.

For expression of the light and/or heavy chains, the expression vector(s) encoding the heavy and/or light chains is introduced into a host cell by standard techniques e.g. electroporation, calcium phosphate precipitation, DEAE-dextran transfection, transduction, infection and the like. Although it is theoretically possible to express the antibodies of the invention in either prokaryotic or eukaryotic host cells, eukaryotic cells are preferred, and most preferably mammalian host cells, because such cells are more likely to assemble and secrete a properly folded and immunologically active antibody. Preferred mammalian host cells for expressing the recombinant antibodies of the invention include Chinese Hamster Ovary (CHO cells) [including dhfr minus CHO cells, as described in Urlaub and Chasin, Proc. Natl. Acad. Sci. USA 77:4216-20, 1980, used with a DHFR selectable marker, e.g. as described in Kaufman and Sharp, J. Mol. Biol. 159:601-21, 1982], NSO myeloma cells, COS cells, and SP2/0 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown under appropriate conditions known in the art. Antibodies can be recovered from the host cell and/or the culture medium using standard purification methods.

The invention provides a host cell comprising a nucleic acid molecule of the present invention. Preferably a host cell of the invention comprises one or more vectors or constructs comprising a nucleic acid molecule of the present invention. For example, a host cell of the invention is a cell into which a vector of the invention has been introduced, said vector comprising a polynucleotide encoding a LCVR of an antibody of the invention and/or a polynucleotide encoding a HCVR of the invention. The invention also provides a host cell into which two vectors of the invention have been introduced; one comprising a polynucleotide encoding a LCVR of an antibody of the invention and one comprising a polynucleotide encoding a HCVR present in an antibody of the invention and each operably-linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes.

Once expressed, the intact antibodies, individual light and heavy chains, or other immunoglobulin forms of the present invention can be purified according to standard procedures of the art, including ammonium sulfate precipitation, ion exchange, affinity (e.g., Protein A), reverse phase, hydrophobic interaction column chromatography, hydroxyapatite chromatography, gel electrophoresis, and the like. Standard procedures for purification of therapeutic antibodies are described, for example, by Feng L1, Joe X. Zhou, Xiaoming Yang, Tim Tressel, and Brian Lee in an article entitled “Current Therapeutic Antibody Production and Process Optimization” (BioProcessing Journal, September/October 2005) (incorporated by reference in its entirety for purposes of teaching purification of therapeutic antibodies). Additionally, standard techniques for removing viruses from recombinantly expressed antibody preparations are also known in the art (see, for example, Gerd Kern and Mani Krishnan, “Viral Removal by Filtration: Points to Consider” (Biopharm International, October 2006)). The effectiveness of filtration to remove viruses from preparations of therapeutic antibodies is known to be at least in part dependent on the concentration of protein and/or the antibody in the solution to be filtered. The purification process for antibodies of the present invention may include a step of filtering to remove viruses from the mainstream of one or more chromatography operations. Preferably, prior to filtering through a pharmaceutical grade nanofilter to remove viruses, a chromatography mainstream containing an antibody of the present invention is diluted or concentrated to give total protein and/or total antibody concentration of about 1 g/L to about 3 g/L. Even more preferably, the nanofilter is a DV20 nanofilter (e.g., Pall Corporation; East Hills, N.Y.). Substantially pure immunoglobulins of at least about 90%, about 92%, about 94% or about 96% homogeneity are preferred, and about 98 to about 99% or more homogeneity most preferred, for pharmaceutical uses. Once purified, partially or to homogeneity as desired, the sterile antibodies may then be used therapeutically, as directed herein.

In view of the aforementioned discussion, the present invention is further directed to an antibody obtainable by a process comprising the steps of culturing a host cell including, but not limited to a mammalian, plant, bacterial, transgenic animal, or transgenic plant cell which has been transformed by a polynucleotide or a vector comprising nucleic acid molecules encoding antibodies of the invention so that the nucleic acid is expressed and, optionally, recovering the antibody from the host cell culture medium.

In certain aspects, the present application provides hybridoma cell lines, as well as to the monoclonal antibodies produced by these hybridoma cell lines. The cell lines disclosed have uses other than for the production of the monoclonal antibodies. For example, the cell lines can be fused with other cells (such as suitably drug-marked human myeloma, mouse myeloma, human-mouse heteromyeloma or human lymphoblastoid cells) to produce additional hybridomas, and thus provide for the transfer of the genes encoding the monoclonal antibodies. In addition, the cell lines can be used as a source of nucleic acids encoding the anti-CCR8 immunoglobulin chains, which can be isolated and expressed (e.g., upon transfer to other cells using any suitable technique (see e.g., Cabilly et al., U.S. Pat. No. 4,816,567; Winter, U.S. Pat. No. 5,225,539)). For instance, clones comprising a rearranged anti-CCR8 light or heavy chain can be isolated (e.g., by PCR) or cDNA libraries can be prepared from mRNA isolated from the cell lines, and cDNA clones encoding an anti-CCR8 immunoglobulin chain can be isolated. Thus, nucleic acids encoding the heavy and/or light chains of the antibodies or portions thereof can be obtained and used in accordance with recombinant DNA techniques for the production of the specific immunoglobulin, immunoglobulin chain, or variants thereof (e.g., humanized immunoglobulins) in a variety of host T-cells or in an in vitro translation system. For example, the nucleic acids, including cDNAs, or derivatives thereof encoding variants such as a humanized immunoglobulin or immunoglobulin chain, can be placed into suitable prokaryotic or eukaryotic vectors (e.g., expression vectors) and introduced into a suitable host T-cell by an appropriate method (e.g., transformation, transfection, electroporation, infection), such that the nucleic acid is operably linked to one or more expression control elements (e.g., in the vector or integrated into the host T-cell genome). For production, host T-cells can be maintained under conditions suitable for expression (e.g., in the presence of inducer, suitable media supplemented with appropriate salts, growth factors, antibiotic, nutritional supplements, etc.), whereby the encoded polypeptide is produced. If desired, the encoded protein can be recovered and/or isolated (e.g., from the host T-cells or medium). It will be appreciated that the method of production encompasses expression in a host T-cell of a transgenic animal (see e.g., WO 92/03918, GenPharm International, published Mar. 19, 1992) (incorporated by reference in its entirety).

Host cells can also be used to produce portions, or fragments, of intact antibodies, e.g., Fab fragments or scFv molecules by techniques that are conventional. For example, it may be desirable to transfect a host cell with DNA encoding either the light chain or the heavy chain of an antibody of this invention. Recombinant DNA technology may also be used to remove some or all the DNA encoding either or both of the light and heavy chains that is not necessary for binding to human CCR8. The molecules expressed from such truncated DNA molecules are also encompassed by the antibodies of the invention.

Methods for expression of single chain antibodies and/or refolding to an appropriate active form, including single chain antibodies, from bacteria such as E. coli have been described and are well-known and are applicable to the antibodies disclosed herein (see, e.g., Buchner et al., Anal. Biochem. 205:263-270, 1992; Pluckthun, Biotechnology 9:545, 1991; Huse et al., Science 246:1275, 1989 and Ward et al., Nature 341:544, 1989, all incorporated by reference herein).

Often, functional heterologous proteins from E. coli or other bacteria are isolated from inclusion bodies and require solubilization using strong denaturants, and subsequent refolding. During the solubilization step, as is well known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena et al., Biochemistry 9:5015-5021, 1970, incorporated by reference herein, and especially as described by Buchner et al., supra.

Renaturation is typically accomplished by dilution (for example, 100-fold) of the denatured and reduced protein into refolding buffer. An exemplary buffer is 0.1 M Tris, pH 8.0, 0.5 M L-arginine, 8 mM oxidized glutathione (GSSG), and 2 mM EDTA.

As a modification to the two-chain antibody purification protocol, the heavy and light chain regions are separately solubilized and reduced and then combined in the refolding solution. An exemplary yield is obtained when these two proteins are mixed in a molar ratio such that a 5-fold molar excess of one protein over the other is not exceeded. Excess oxidized glutathione or other oxidizing low molecular weight compounds can be added to the refolding solution after redox shuffling is completed.

In addition to recombinant methods, the antibodies, labeled antibodies and antigen-binding fragments thereof that are disclosed herein can also be constructed in whole or in part using standard peptide synthesis. Solid phase synthesis of the polypeptides of less than about 50 amino acids in length can be accomplished by attaching the C-terminal amino acid of the sequence to an insoluble support followed by sequential addition of the remaining amino acids in the sequence. Techniques for solid phase synthesis are described by Barany & Merrifield, The Peptides: Analysis, Synthesis, Biology. Vol. 2: Special Methods in Peptide Synthesis, Part A. pp. 3-284; Merrifield et al., J. Am. Chem. Soc. 85:2149-2156, 1963, and Stewart et al., Solid Phase Peptide Synthesis, 2nd ed., Pierce Chem. Co., Rockford, Ill., 1984. Proteins of greater length may be synthesized by condensation of the amino and carboxyl termini of shorter fragments. Methods of forming peptide bonds by activation of a carboxyl terminal end (such as by the use of the coupling reagent N,N′-dicylohexylcarbodimide) are well known in the art.

The following examples are offered to more fully illustrate the invention but are not construed as limiting the scope thereof.

### Example 1

### Generation of Mouse Monoclonal Antibodies Targeting Specifically to Human CCR8

Human CCR8 expression plasmids were constructed, and CHO-K1 cells overexpressing hCCR8 were created to use as immunogens for generating anti-hCCR8 monoclonal antibodies. BALB/c, C57BL/6, A/J, and SJL mice were each immunized bi-weekly (6 or more total immunizations) with plasmid DNA harboring hCCR8, hCCR8-expressing cells or membrane protein. Immunogen preparations were injected subcutaneously or intraperitoneally. Sera from immunized mice were collected and tested by flow cytometry on both hCCR8-expressing CHO-K1 cells and parental CHO-K1 cells. The mice with a significant level (titer) of antibody against hCCR8 were selected for hybridoma fusion. Briefly, splenocytes were harvested 3-4 days after the final immunization for fusion with myeloma cell line SP2/0 from ATCC (American Type Culture Collection). Electric fusion methods were used to obtain hybridoma cells.

The hybridoma supernatants were screened for antigen binding by flow cytometry and/or cell-based ELISA. In the cell-based ELISA, both CHO-K1 cells and hCCR8-expressing cells were incubated with the supernatants. The cells were washed with PBS and then incubated with goat anti-mouse IgG-HRP secondary antibody. TMB was added after the cells were washed. The absorbance readings were taken at 450 nm by a plate reader. The primary hybridoma clones with the highest ratios of OD450 in hCCR8-expressing cells to CHO-K1 cells were selected for subcloning by the limiting dilution method.

The subclone supernatants were tested by cell-based ELISA to confirm the presence of antibody that specifically binds hCCR8. Only subclones with the ratio of OD450 higher than 1.9 were selected for further analysis.

Human CCL1 (hCCL1) is the dominant ligand of CCR8. The subclone supernatants were tested in a cell-based CCR8 CHO-K1 β-arrestin bioassay (Eurofins) to determine antagonist activity of anti-CCR8 antibody, i.e., blockade of CCR8 downstream signaling induced by hCCL1. Briefly, approximately 10000 cells were seeded into wells in a 96-well plate and incubated at 37° C., 5% CO₂ for 24-48 hours. Supernatants were added and incubated for 30 minutes to allow antibody to bind to hCCR8 on the cells. Following the addition of 2 nM of hCCL1, the plate was incubated at 37° C., 5% CO₂ for 90 minutes to stimulate β-arrestin production. Finally, the detection solution was added and the plate was incubated in the dark at room temperature for 1 hour. The plate was read on a Varioskan LUX Multimode Microplate Reader (Thermo Fisher Scientific). The subclones that showed at least 50% inhibition of β-arrestin production in the assay were selected for sequencing (see Table 3).

Total RNA was isolated from the hybridoma cells following the manufacturer's instructions (Vazyme). Total RNA was then reverse-transcribed into cDNA using either isotype-specific antisense primers or universal primers following the technical manual of SMARTScribe Reverse Transcriptase (TaKaRa). Antibody fragments of heavy chain and light chain were amplified according to the standard operating procedure of rapid amplification of cDNA ends (RACE) of ProBio. Amplified antibody fragments were cloned into a standard cloning vector separately. Colony PCR was performed to screen for clones with inserts of correct sizes and sequenced. Mouse mAb clones listed in Table 3 comprise the heavy chain variable region (VH), and/or the light chain variable region (VL), and/or the CDR sequences set forth in SEQ ID NOS: 3-25 and 35-77.

### Example 2

### Anti-hCCR8 Antibodies Block hCCL1 Binding to hCCR8

Mouse antibodies 41E1C2A5, 46A5C4B1 and 80E4D1F11, and chimeric antibodies (human IgG1), 504E12D8D12, 516D7D12, 525F2F3F11 and 531B9B1C9 were assessed in the CCR8 CHO-K1 β-arrestin assay to determine the antagonist activity in blocking hCCL1-induced activation of CCR8 signaling. The data was plotted using GraphPad Prism version 9 (San Diego, CA) and IC50 was determined by nonlinear regression curve fit and summarized in Table 6.

TABLE 6
Antagonist activity of anti-hCCR8 antibodies
measured by β-arrestin assay

| mAb clone | IC50 (nM) |
|---|---|
| 41E1C2A5 | 4.684 |
| 46A5C4B1 | 6.237 |
| 80E4D1F11 | 7.105 |
| 504E12D8D12 | 0.867 |
| 516D7D12 | 1.514 |
| 525F2F3F11 | 1.519 |
| 531B9B1C9 | 2.182 |

### Example 3

### Anti-hCCR8 Antibodies do not Bind to Human CCR4

Mouse antibodies 41E1C2A5, 46A5C4B1 and 80E4D1F11, and chimeric antibodies 504E12D8D12, 516D7D12, 525F2F3F11 and 531B9B1C9 bind specifically to hCCR8-expressing CHO-K1 cells and block hCCL1 binding to hCCR8, indicating that they are CCR8 specific antibodies. The antibodies were further screened for binding to human CCR4 (hCCR4) by flow cytometry. The antibodies were incubated with CHO-K1 cells and hCCR4-expressing CHO-K1 cells for 60 minutes at 4° C. The cells were washed thoroughly with PBS buffer and were then incubated with FITC-conjugated goat anti-mouse IgG Fc antibody or anti-human IgG Fc antibody. Mean Fluorescence Intensity (MFI) of antibody binding on the cells were analyzed by flow cytometry (Table 7). The results showed that the anti-hCCR8 antibodies do not bind to hCCR4.

TABLE 7
MFI of antibody binding measured by flow cytometry

| mAb clone | CHO-K1 | CHO-K1/hCCR4 |
|---|---|---|
| 41E1C2A5 | 17.8 | 17.9 |
| 46A5C4B1 | 47.9 | 58.2 |
| 80E4D1F11 | 20.1 | 20.6 |
| 504E12D8D12 | 17.5 | 23.3 |
| 516D7D12 | 14.3 | 20.3 |
| 525F2F3F11 | 15.5 | 23.2 |
| 531B9B1C9 | 12.0 | 13.3 |

### Example 4

### ADCC Activity of Anti-hCCR8 Chimeric Antibodies

The ADCC activity of anti-hCCR8 chimeric antibodies were measured by a reporter bioassay (BPS Bioscience). Briefly, hCCR8-expressing CHO-K1 cells were seeded in a 96-well assay plate at a density of 12,000 cells/well in 100 μl assay medium and incubated at 37° C., 5% CO₂ overnight. After the medium was discarded, 60 μl of serially diluted anti-CCR8 antibody was added and incubated for 1 hour. 40 μl of ADCC/NFAT-reporter-Jurkat cells (˜75,000 cells) was added. After incubation for 5-6 hours. 100 μl of luciferase substrate was added to each well and the plate was gently rocked for 15 minutes to 1 hour at room temperature. Luminescence was measured on a Varioskan LUX Multimode Microplate Reader (Thermo Fisher Scientific). Each treatment was run in triplicate. Data analysis was performed using GraphPad Prism (version 9) to determine EC50.

The results showed that the murine-human chimeric (human IgG1) antibodies 41E1C2A5, 504E12D8D12 and 531B9B1C9 had potent ADCC activity (Table 8).

TABLE 8
EC50 of ADCC activity of chimeric anti-hCCR8 antibodies

| mAb | IC50 (nM) |
|---|---|
| 41E1C2A5 | 0.049 |
| 504E12D8D12 | 0.015 |
| 531B9B1C9 | 0.028 |

### Example 5

### Humanization of Mouse Anti-hCCR8 Antibodies

### Humanization of Mouse Anti-hCCR8 mAb 41E1C2A5

The mouse anti-hCCR8 mAb 41E1C2A5 was humanized by CDR grafting and back mutation. The structure of the parental antibody was modeled by computer-aided homology modeling program (MOE). The CDRs were grafted into the frameworks of the most closely related human germlines based on sequence similarity. The human germline IGHV3-73*01 was selected for the heavy chain and IGKV2-28*01 for the light chain. Several residues in the frameworks of the heavy and light chains were mutated back to corresponding residues in the mouse antibody to preserve antibody structure.

The heavy and light chains were designed and paired with each other to produce antibodies by transient expression for affinity ranking by flow cytometry. Briefly, 50 μl of hCCR8-expressing CHO-K1 cells (1×10⁵ cells/well) were loaded onto a 96-well plate. A 3-fold dilution series (11 points) of each antibody was prepared with the final starting concentration of 45 μg/ml. The antibodies were incubated with the cells at 4° C. for 1 hour. After thoroughly washing, the cells were incubated with Alexa Fluor 647-conjugated goat anti-human IgG (H+L) antibody. Geometric means were measured by flow cytometry. A nonlinear regression curve fit (4PL) was used to generate sigmoidal curve to produce EC50 in GraphPad Prism. Humanized antibodies 41E1C2A5-HC3+LC4 (SEQ ID NO: 86 and SEQ ID NO: 87) and 41E1C2A5-HC4+LC2 (SEQ ID NO: 88 and SEQ ID NO: 89) have comparable binding affinity as chimeric 41E1C2A5 mAb.

### Humanization of Mouse Anti-hCCR8 mAb 504E12D8D12

The mouse anti-hCCR8 mAb 504E12D8D12 was humanized by grafting CDRs into the framework of the closest human germline IGHV3-73*01 for the heavy chain and IGKV2-18*01 for the light chain. Back mutations were made in the framework sequences. In the light chain CDR1 of 504E12D8D12, there is a potential deamidation motif NG. The mutations N33Q and G34A were made to eliminate potential deamidation liability. The heavy and light chains were designed and paired to produce antibodies for affinity ranking. Humanized antibodies 504E12D8D12-HC1+LC1 (SEQ ID NO: 90 and SEQ ID NO: 91) and 504E12D8D12-HC1+LC1(G34A) (SEQ ID NO: 90 and SEQ ID NO: 92) had comparable binding affinity as chimeric 504E12D8D12 mAb.

### Example 6

### Humanized Antibodies Block Calcium Flux Mediated by hCCR8

Humanized antibodies were tested by FLIPR calcium flux assay to assess their antagonist activity in blocking the hCCR8-mediated calcium flux induced by hCCL1. Briefly, hCCR8-expressing CHO-K1 cells (ProBio) were seeded onto a 384-well assay plate and incubated at 37° C., 5% CO₂ for 16-20 hours. After incubation the plate was placed at room temperature. The dye-loading solution (FLIPR Calcium 4 assay kit, Molecular Devices) was prepared with GPCR buffer and 20 μl was transferred into each well. Serial dilutions of antibodies were prepared and 10 μl was added. After incubation at 37° C., 5% CO₂ for 1 hour, hCCL1 was added into each well so that the final concentration was equal to the EC80. Fluorescence signal was monitored using the FLIPR Tetra system. Anti-hCCR8 antibodies (Reference Ab #1 and Reference Ab #2) described in the literature were also tested in the assay. Data was recorded and analyzed using ScreenWorks (version 3.1) program. A nonlinear regression curve fit (4PL) was used to generate sigmoidal curve to calculate IC50 in GraphPad Prism.

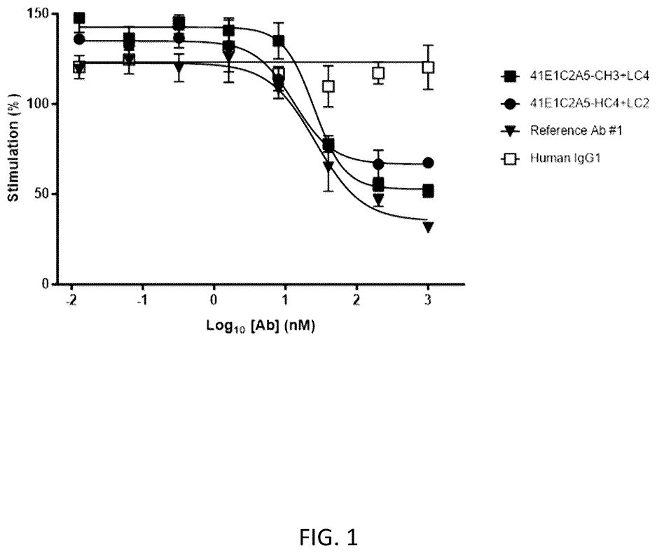

The dose response curves of humanized antibodies 41E1C2A5-HC3+LC4 and 41E1C2A5-HC4+LC2 are shown in FIG. 1 and the IC50 values are summarized in Table 9.

TABLE 9
Antagonist activity of humanized mAbs derived
from 41E1C2A5 measured by calcium flux assay

| mAb | IC50 (nM) |
|---|---|
| 41E1C2A5-HC3 + LC4 | 25.3 |
| 41E1C2A5-HC4 + LC2 | 13.1 |
| Reference Ab #1 | 26.3 |

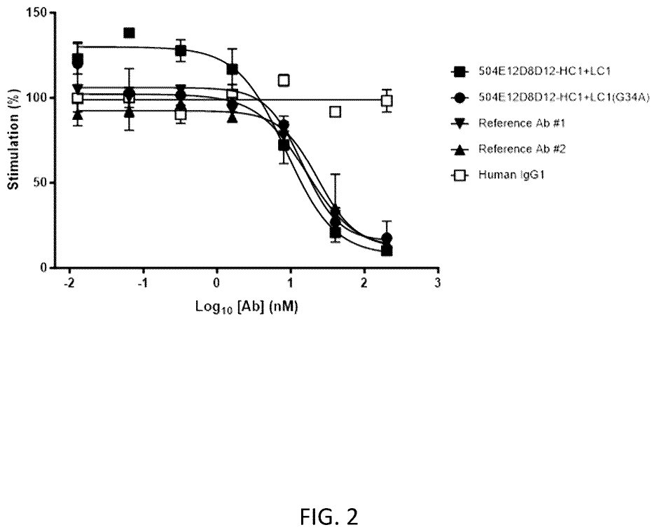

The dose response curves of humanized antibodies 504E12D8D12-HC1+LC1 and 504E12D8D12-HC1+LC1 (G34A) are shown in FIG. 2 and the IC50 values are summarized in Table 10. 504E12D8D12-HC1+LC1 and 504E12D8D12-HC1+LC1 (G34A) had stronger or similar antagonist activity compared to the reference antibodies.

TABLE 10
Antagonist activity of humanized mAbs derived
from 504E12D8D12 measured by calcium flux assay

| mAb | IC50 (nM) |
|---|---|
| 504E12D8D12-HC1 + LC1 | 8.6 |
| 504E12D8D12-HC1 + LC1(G34A) | 14.4 |
| Reference Ab #1 | 16.1 |
| Reference Ab #2 | 23.1 |

### Example 7

### Humanized Antibodies Bind to hCCR8 with High Affinity

Humanized antibodies 504E12D8D12-HC1+LC1 and 504E12D8D12-HC1+LC1 (G34A) were tested by the Kinetic Exclusion Assay (KinExA, Sapidyne Instruments) to determine the affinity of binding to hCCR8-overexpressing cells. Briefly, polystyrene particles (#442178, Sapidyne Instruments) were coated with goat anti-human IgG Fc-specific Fab fragment (#109-007-008, Jackson ImmunoResearch Lab) according to the manufacturer's protocol. The antibodies were incubated with serial dilutions of hCCR8-expressing HEK293 cells at room temperature overnight by gently rotating to achieve equilibrium. The final antibody concentrations were adjusted to 0.05 nM and 1 nM, respectively. The supernatants were collected and run on KinExA 3200 (Sapidyne Instruments), and the antibody was captured by the coated polystyrene particles. The captured antibody was detected with Alexa Fluor 647-conjugated goat anti-human IgG (H+L) antibody (#109-605-003, Jackson ImmunoResearch Lab). Data was recorded and analyzed using KinExA Pro software (version 4.3.20).

Humanized antibodies 504E12D8D12-HC1+LC1 and 504E12D8D12-HC1+LC1 (G34A) were found to have high binding affinity for hCCR8 (Table 11).

TABLE 11
Dissociation constant of humanized
antibodies determined by KinExA

| mAb | K_{D} (nM) | 95% confidence interval |
|---|---|---|
| 504E12D8D12-HC1 + LC1 | 0.072 | 0.029-0.143 |
| 504E12D8D12-HC1 + LC1(G34A) | 0.072 | 0.036-0.127 |
| Reference Ab #1 | 0.079 | 0.036-0.144 |

### Example 8

### Bispecific Antibodies Targeting CCR8 and CTLA-4

### Construction of Bispecific Antibodies Targeting CCR8 and CTLA-4

A heavy chain designated FP578-HC (SEQ ID NO: 93) was prepared by fusing a humanized anti-hCTLA-4 single domain antibody (sdAb) (amino acid residues 470-599 of SEQ ID NO: 93) to the C-terminus of heavy chain 504E12D8D12-HC1 (amino acid residues 1-454 of SEQ ID NO: 93) by a linker (amino acid residues 455-469 of SEQ ID NO: 93). FP578-HC was then paired with 504E12D8D12-LC1 (G34A) (SEQ ID NO: 92) to produce the bispecific antibody FP578-01. FP578-HC was also paired with 504E12D8D12-LC1 (SEQ ID NO: 91) to produce the bispecific antibody FP578-02, respectively.

Bispecific Antibodies can Bind to CTLA-4 while Bound to CCR8

Bispecific antibodies FP578-01 and FP578-02 and anti-hCCR8 antibodies 504E12D8D12-HC1+LC1 (G34A) and 504E12D8D12-HC1+LC1 were incubated with hCCR8-expressing HEK293 cells for 1 hour at 4° C. The cells were washed and then incubated with biotinylated recombinant human CTLA-4-Fc chimera (#786704, BioLegend) for 1 hour at 4° C. After the cells were thoroughly washed, CTLA-4 was detected with phycoerythrin (PE)-conjugated streptavidin (#405203, BioLegend) by flow cytometry.

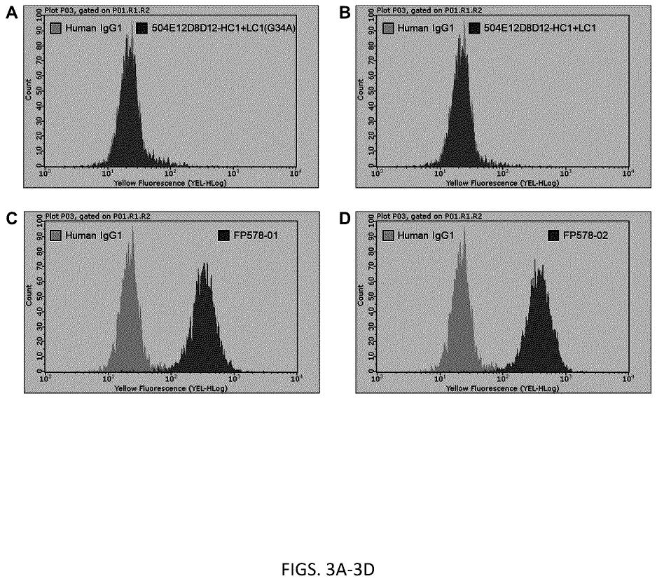

The results demonstrate that both bispecific antibodies FP578-01 and FP578-02 can bind CTLA-4 while concurrently bound to hCCR8 on hCCR8-expressing HEK293 cells. No specific binding of the anti-hCCR8 antibodies to CTLA-4 was observed (FIG. 3).

Bispecific Antibodies have High Binding Affinity to hCCR8

The affinity of bispecific antibodies FP578-01 and FP578-02 were determined by the kinetic exclusion assay as described above. The data showed that K_{D} of the bispecific antibodies and the anti-CCR8 antibodies are comparable (Table 12).

TABLE 12
Affinity of bispecific antibodies determined by KinExA

| Bispecific Ab | K_{D} (nM) | 95% confidence interval |
|---|---|---|
| FP578-01 | 0.102 | 0.031-0.249 |
| FP578-02 | 0.104 | 0.022-0.258 |

Bispecific Antibodies Block hCCL1 Binding to hCCR8

Bispecific antibodies FP578-01 and FP578-02 were tested in the cell-based CCR8 CHO-K1 β-arrestin assay as described above to determine antagonist activity. The data showed that the potency of FP578-01 and FP578-02 are comparable to the anti-hCCR8 antibodies in blocking hCCR8 downstream signaling induced by hCCL1 (Table 13).

TABLE 13
Antagonist activity of bispecific antibodies
measured by β-arrestin assay

| Bispecific Ab | IC50 (nM) |
|---|---|
| FP578-01 | 4.263 |
| FP578-02 | 3.602 |

All of the articles and methods disclosed and claimed herein can be made and executed without undue experimentation in light of the present disclosure. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1-29. (canceled)

30. An isolated antibody, or antigen-binding fragment thereof, which specifically binds human C—C Chemokine Receptor Type 8 (CCR8) and comprises either: (a) the heavy chain CDR1, CDR2, and CDR3 sequences set forth in SEQ ID NO: 3, SEQ ID NO: 4, and SEQ ID NO: 5; and the light chain CDR1, CDR2, and CDR3 sequences set forth in SEQ ID NO: 12, SEQ ID NO: 13, and SEQ ID NO: 14; or (b) the heavy chain CDR1, CDR2, and CDR3 sequences set forth in SEQ ID NO: 7, SEQ ID NO: 37, and SEQ ID NO: 42; and the light chain CDR1, CDR2, and CDR3 sequences set forth in SEQ ID NO: 12, SEQ ID NO: 13, and SEQ ID NO: 14.

31. An isolated antibody, or antigen-binding fragment thereof according to claim 1, which comprises: (a) the heavy chain CDR1, CDR2, and CDR3 sequences set forth in SEQ ID NO: 3, SEQ ID NO: 4, and SEQ ID NO: 5; and the light chain CDR1, CDR2, and CDR3 sequences set forth in SEQ ID NO: 12, SEQ ID NO: 13, and SEQ ID NO: 14;

32. An isolated antibody, or antigen-binding fragment thereof according to claim 1, which comprises: (a) the heavy chain CDR1, CDR2, and CDR3 sequences set forth in SEQ ID NO: 7, SEQ ID NO: 37, and SEQ ID NO: 42; and the light chain CDR1, CDR2, and CDR3 sequences set forth in SEQ ID NO: 12, SEQ ID NO: 13, and SEQ ID NO: 14.

33. An isolated antibody, or antigen-binding fragment thereof according to claim 1, which comprises either: (a) the heavy chain variable region having the amino acid sequence set forth in SEQ ID NO: 18 and the light chain variable region having the amino acid sequence set forth in SEQ ID NO: 19; or (b) the heavy chain variable region having the amino acid sequence set forth in SEQ ID NO: 66 and the light chain variable region having the amino acid sequence set forth in SEQ ID NO: 76.

34. The isolated antibody or antigen-binding fragment thereof according to claim 1, further comprising a set of four variable region framework regions from a human immunoglobulin (IgG).

35. The isolated antibody or antigen-binding fragment thereof according to claim 1 that binds to CCR8 protein with a dissociation constant (K_{D}) of at least about 1×10⁻⁶ M, at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M.

36. The isolated antibody or antigen-binding fragment thereof according to claim 1, wherein the antibody or antigen-binding fragment is selected from a human antibody, a humanized antibody, chimeric antibody, a monoclonal antibody, a polyclonal antibody, a recombinant antibody, an antigen-binding antibody fragment, a single chain antibody, a diabody, a triabody, a tetrabody, a Fab fragment, a Fab′ fragment, a Fab₂ fragment, a F(ab)′₂ fragment, an IgD antibody, an IgE antibody, an IgM antibody, an IgG1 antibody, an IgG2 antibody, an IgG3 antibody, and an IgG4 antibody.

37. An isolated humanized antibody or antigen-binding fragment thereof according to claim 36 which comprises the heavy chain sequence selected from the group of amino acid sequences defined by SEQ ID NOS: 86, 88 and 90; and the light chain sequence selected from the group of amino acid sequences defined by SEQ ID NOS: 87, 89 and 91-92.

38. The isolated humanized or human monoclonal antibody or antigen-binding fragment according to claim 37 comprising the heavy chain sequence of SEQ ID NO: 86 and the light chain sequence of SEQ ID NO: 87.

39. The isolated humanized or human monoclonal antibody or antigen-binding fragment according to claim 37 comprising the heavy chain sequence of SEQ ID NO: 88 and the light chain sequence of SEQ ID NO: 89.

40. The isolated humanized or human monoclonal antibody or antigen-binding fragment according to claim 37 comprising the heavy chain sequence of SEQ ID NO: 90 and the light chain sequence of SEQ ID NO: 91.

41. The isolated humanized or human monoclonal antibody or antigen-binding fragment according to claim 37 comprising the heavy chain sequence of SEQ ID NO: 90 and the light chain sequence of SEQ ID NO: 92.

42. A pharmaceutical composition comprising an isolated antibody, or antigen-binding fragment thereof according to claim 30 in admixture with a pharmaceutically acceptable carrier.

43. An isolated immunoconjugate comprising an antibody or antigen-binding fragment thereof according to claim 30 coupled to an effector molecule, wherein the effector molecule is selected from the group consisting of an immunotoxin, a cytokine, a chemokine, a therapeutic agent, and a chemotherapeutic agent.

44. A pharmaceutical composition comprising an immunoconjugate according to claim 43 in admixture with a pharmaceutically acceptable carrier.

45. A bispecific antibody comprising an antibody or antigen-binding fragment thereof according to claim 1 coupled to an antibody or antigen-binding fragment thereof that specifically binds Cytotoxic T-Lymphocyte Antigen-4 (CTLA-4), wherein the bispecific antibody comprises the heavy chain of SEQ ID NO: 93 and the light chain of SEQ ID NO: 92; or (b) comprises the heavy chain of SEQ ID NO: 93 and the light chain of SEQ ID NO: 91.

46. A bispecific antibody according to claim 45 which comprises the heavy chain of SEQ ID NO: 93 and the light chain of SEQ ID NO: 92.

47. A bispecific antibody according to claim 45 which comprises the heavy chain of SEQ ID NO: 93 and the light chain of SEQ ID NO: 91.

48. A pharmaceutical composition comprising a bispecific antibody according to claim 43 in admixture with a pharmaceutically acceptable carrier.

49. A method of treating a subject suffering from a CCR8-associated disorder, comprising administering to said subject a therapeutically effective amount of a pharmaceutical composition according to claim 42.
